SEQUENCE LISTING (9 sequences)

SEQ ID NO: 1 - protein, 504 aa, Artificial Sequence [sequence omitted]

SEQ ID NO: 2 - DNA, 1515 bp, Artificial Sequence
  DNA coding for the Human Glycoprotein VI- Fc fusion protein of SEQ ID No 1
atgtctccatccccgaccgccctcttctgtcttgggctgtgtctggggcgtgtgccagcgcagagtggaccgctccccaagccctccctccaggctctgcccagctccctggtgcccctggagaagccagtgaccctccggtgccagggacctccgggcgtggacctgtaccgcctggagaagctgagttccagcaggtaccaggatcaggcagtcctcttcatcccggccatgaagagaagtctggctggacgctaccgctgctcctaccagaacggaagcctctggtccctgcccagcgaccagctggagctcgttgccacgggagtttttgccaaaccctcgctctcagcccagcccggcccggcggtgtcgtcaggaggggacgtaaccctacagtgtcagactcggtatggctttgaccaatttgctctgtacaaggaaggggaccctgcgccctacaagaatcccgagagatggtaccgggctagtttccccatcatcacggtgaccgccgcccacagcggaacctaccgatgctacagcttctccagcagggacccatacctgtggtcggcccccagcgaccccctggagcttgtggtcacaggaacctctgtgacccccagccggttaccaacagaaccaccttcctcggtagcagaattctcagaagccaccgctgaactgaccgtctcattcacaaacaaagtcttcacaactgagacttctaggagtatcaccaccagtccaaaggagtcagactctccagctggtcctgcccgccagtactacaccaagggcaacggcggccgcgagtccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 490 aa, Artificial Sequence
  Human Glycoprotein VI-Fc fusion protein
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNGGRPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 37 bp, Artificial Sequence
  Synthetic PCR primer
cgcggggcggccgcgagtccaaatcttgtgacaaaac

SEQ ID NO: 5 - DNA, 33 bp, Artificial Sequence
  Synthetic PCR primer
gcgggaagctttcatttacccggagacagggag

SEQ ID NO: 6 - DNA, 36 bp, Artificial Sequence
  Synthetic PCR primer
gcggggagatctaccaccatgtctccatccccgacc

SEQ ID NO: 7 - DNA, 34 bp, Artificial Sequence
  Synthetic PCR primer
cgcggggcggccgccgttgcccttggtgtagtac

SEQ ID NO: 8 - DNA, 32 bp, Artificial Sequence
  Synthetic PCR primer
cgcggggcggccgcccagcacctgaactcctg

SEQ ID NO: 9 - DNA, 34 bp, Artificial Sequence
  Synthetic PCR primer
cgcggggatatctcatttacccggagacagggag

(19) United States
(12) Patent Application Publication
(10) Pub. No.: US 2009/0130021 A1
(43) Pub. Date: May 21, 2009

(54) Methods, products and uses involving platelets and/or the vasculature

(76) Inventors: Gotz Munch, Munchen (DE); Andreas Bultmann, Planegg (DE); Oliver Vimpany Arnold Boucher, London (GB); Suresh Babubhai Chahwala, London (GB); Meinrad Gawaz, Tubingen (DE); Martin Ungerer, Grafelfing (DE)

Correspondence Address: KLARQUIST SPARKMAN, LLP, 121 SW SALMON STREET, SUITE 1600, PORTLAND, OR, 97204, US

(21) Appl. No.: 11/792,857

(22) PCT Filed: Dec. 12, 2005
(86) PCT No.: PCT/GB05/04764

§ 371 Date: Apr. 1, 2008

Related U.S. Application Data
(63) Continuation-in-part of application No. 11/009,106, filed on Dec. 10, 2004.
Application No. 11/009,106 is a continuation-in-part of application No. 10/489,053, filed on Sep. 24, 2004, now Pat. No. 7,514,543.

(30) Foreign Application Priority Data
Jun. 7, 2002 (EP) 02 012 742.9
Nov. 11, 2005 (EP) 05256993.6

(51) Int. Cl. A61K 49/00; A61K 38/02; A61K 39/395; A61K 38/17; A61P 9/10; A61K 9/00; A61F 2/04

(52) U.S. Cl. 424/9.1; 514/2; 424/130.1; 514/12; 424/400; 424/423

(57) ABSTRACT

The present disclosure relates to agents which interfere with the binding of GPVI to various components. Agents which interfere with GPVI interaction with one or both of fibronectin and vitronectin or sequences thereof are also disclosed. Methods of treating disorders or diseases which involve pathological, dysfunctional or non-pathological interaction of GPVI with fibronectin and/or vitronectin are included in the present disclosure. The invention also relates to uses of agents for the prevention or treatment of disorders arising from blood platelet adhesion and aggregation.

### FIELD OF THE DISCLOSURE

The present disclosure relates in some aspects to agents which can interfere with the binding of GPVI to various components. Methods of treating disorders or diseases which involve pathological, dysfunctional or non-pathological interactions of GPVI and/or the vasculature are included in the present disclosure. The disclosure also relates to uses of agents for the prevention or therapy of disorders which directly or indirectly involve platelets, as well as other subject matter.

### BACKGROUND

Platelet adhesion to the endothelium plays a crucial role in the pathophysiology of reperfusion, sepsis, and cardiovascular diseases (1, 2, 3, 4). Endothelial cell dysfunction allows platelet adhesion even without exposure of extracellular matrices (1, 5). Similar to the recruitment of leukocytes⁶, the adhesion of platelets to the endothelial surface is a multistep process, in which platelets are tethered to the vascular wall, followed by subsequent firm adhesion of the platelets. Whereas the adhesion receptors involved in platelet attachment to the subendothelial matrix have been well defined^{1,5}, the molecular basis of the regulation of the interaction between platelets and the intact vascular endothelium are incompletely understood.

At sites of arterial injury the GPVI-collagen interaction is a major trigger of platelet activation and primary Hemostasis; GPVI binding to collagen results in platelet signaling and activation of β₁- and β₃-integrins, a critical step for stable platelet adhesion to extracellular matrices¹⁰.

Glycoprotein VI (GPVI) is a 60-65 kDa type I transmembrane glycoprotein, which belongs to the immunoglobulin superfamily (Clemetson, J. M., eta/(1999) J Biol. Chem. 274, 29019-29024; Jandrot-Perrus, M., Busfield, et al (2000). Blood 96, 1798-1807; Gibbins, 3. M., et a/(1997). FEBS Lett. 413, 255-259; Zheng, Y. M., et al (2001). J Biol. Chem. 276, 12999-13006; Suzuki-Inoue, K., Tulasne, D., et al. (2002) J Biol. Chem.; Barnes, M. 3., Knight, C. G., Farndale, R. W. (1998) Curr. Opin. Hematol. 5, 314-320; Falet, H., et a/(2000) Blood 96, 3786-3792; Pasquet, 3. M., et al (1999) Mol. Cell Biol. 19, 8326-8334; Berlanga, O., eta/(2002) Eur. J. Biochem. 269, 2951-2960).

Platelets deficient in GPVI show loss of collagen-induced adhesion and aggregation in vitro (Sugiyama, T., et al (1987) Blood 69, 1712-1720). Likewise, function blocking anti-GPVI monoclonal antibodies attenuate ex vivo platelet aggregation in response to collagen and collagen-related peptide (CRP), which mimics collagen triple helix (Suglyama, T., Ishibashi, T., Okuma, M. (1993) Int. J. Hematol. 58, 99-104; Schulte, V., et al (2001) J Biol. Chem. 276, 364-368). Only recently has it been shown with in vivo evidence that GPVI may in fact strictly be required in the process of platelet recruitment under physiological shear stress following vascular injury. In different mouse models of endothelial denudation both the inhibition or absence of GPVI virtually abolished platelet-vessel wall interactions and platelet aggregation, thereby indicating GPVI as the major determinant of arterial thrombus formation (Massberg, S., et al. (2003) J. Exp. Med. 197, 41-49).

Activated endothelial cells express a variety of adhesion receptors (e.g. β₃-integrin, P-selectin, ICAM-1) (13-21) and molecules (e.g. von Willebrand factor (vWF), vitronectin (Vn), fibronectin (Fn)) (22-24) that have been shown to play a role in platelet/endothelium adhesion. Most of these adhesion molecules are presented in substantial amounts on the luminal aspect of endothelial cells.

Fibronectin is a large multidomain glycoprotein found in connective tissue, on cell surfaces, and in plasma and other body fluids. It interacts with a variety of macromolecules including components of the cytoskeleton and the extracellular matrix, fibrinolytic, acute phase and complement systems, and with cell-surface receptors on a variety of cells including fibroblasts, neurons, phagocytes and bacteria. It is composed of two peptide chains of approximately 275,000 molecular weight which are linked through two interchain disulfide bonds at the COOH-terminal end of the molecule.

Vitronectin is a mixture of two monomeric glycoproteins (65 and 75 kD) present in blood and the extracellular matrix (ECM) of many tissues. Vitronectin and fibronectin are the two major adhesive proteins in plasma and serum. Like many other adhesion molecules, vitronectin binds to cells through an interaction of the Arg-Gly-Asp (RGD) sequence in its cell binding domain with vitronectin-specific cell surface receptors, such as integrins αᵥβ₃ and αᵥβ₅.

Previously, it was shown that resting platelets recognize distinct adhesion receptors exposed on activated endothelium including P-selectin and β₃-integrins (3). However, the molecular mechanisms that initiate platelet activation and stable platelet attachment to activated endothelium have not been elucidated.

Platelets have been shown to be involved in the development of thromboembolic complications of advanced atherosclerotic lesions. However, there is also evidence to suggest that platelets are also involved in the initiation and progression of atherosclerotic plaques. However, the molecules involved and roles thereof have not yet been elucidated in great detail.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### BRIEF SUMMARY OF THE DISCLOSURE

The present application discloses amongst other things products, for example molecules, which bind to or interact with plural vascular domains or binding sites. Each domain may comprise a different ligand, for example adhesion ormatrix protein, from each other domain. In embodiments, at least one of the domains binds to or interacts with GPVI. In one class of products, all the binding sites bind to or interact with GPVI. All the domains or binding sites may simultaneously be exposed on a dysfunctional or diseased area of blood vessel wall, for example an atherosclerotic or inflamed area. Particular embodiments relate to products which are capable of binding to a dysfunctional or inflamed area of blood vessel wall which does not include an atherosclerotic plaque or lesion.

In one aspect, there is disclosed an agent which is capable of binding to a plurality of GPVI-binding sites, optionally a plurality of types of GPVI-binding sites, for example of a dysfunctional, inflamed or atherosclerotic blood vessel area, for example a human blood vessel area. The agent optionally has one or more of the following three capabilities:
1. It is capable of inhibiting collagen-induced ATP release from human platelets by at least 65%;
2. it is capable of inhibiting thrombus formation caused by induction of arterial lesions in the mouse by at least 70% as compared with a control administered with an Fc polypeptide, the relative inhibitions of thrombus formation being determined by measurement of the en face thrombus size as a percentage of an artery wall area (e.g. a human artery wall area) placed on a surface for the purpose of said measurement;
3. It is capable of inhibiting platelet aggregation in response to collagen by at least 50%.

The agent may be capable of inhibiting the binding of GPVI to collagen and a protein selected from fibronectin, vitronectin and combinations thereof.

Included in the disclosure is the interaction of GPVI with substances previously unknown to interact with GPVI, particularly fibronectin and vitronectin. Thus, there are disclosed herein previously unknown GPVI ligands, and complexes of GPVI-fibronectin, GPVI vitronectin and GPVI-fibronectin-vitronectin. There is further disclosed a role of GPVI binding to fibronectin in platelet adhesion. Additionally disclosed is a role of GPVI binding to vitronectin in platelet adhesion. The present invention discloses uses of agents which prevent or inhibit the interaction of GPVI with the novel ligands. Methods of treatment utilising the agents are disclosed. Assays utilising the novel ligands of GPVI are included in the disclosure.

The present specification discloses the role of GPVI interaction with vitronectin and/or fibronectin in the mediation of platelet adhesion to endothelium in vitro and in vivo. The present specification discloses the role of GPVI interaction with exposed sub-endothelial matrix and/or adhesion proteins other than collagen.

This specification contains data demonstrating that inhibition of GPVI binding to fibronectin and/or vitronectin reduces platelet/endothellium adhesion and, as a consequence, vascular remodelling during atherogenesis in vivo. The data indicate that inhibitors of the interaction between GPVI, on the one hand, and fibronectin and/or vitronectin on the other will have use in the prevention of atherogenesis and the progression of atheroscleroma. Such inhibitors will have use in preventing initiation of atherosderoma or its progression, inter alia, in patients free of active atherosclerotic lesions and/or in chronic therapy.

The disclosure accordingly includes an agent which is capable of inhibiting GPVI interaction with a molecule selected from the group consisting of vitronectin, fibronectin and combinations thereof. In embodiments, the agent has one or more of the three capabilities enumerated above. The agent may additionally be capable of inhibiting GPVI interaction with collagen. In all aspects, the disclosure includes, but is not necessarily limited to, embodiments in which at least one of collagen, fibronectin and vitronectin are human.

In certain embodiments, the agents of the disclosure are not a fusion protein described in WO 03/104282. Accordingly, the disclosure includes embodiments in which the agent is not a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

Particular agents of the disclosure comprise an amino acid sequence of or comprised in an extracellular domain of GPVI, or a variant thereof that is functional for binding to at least one protein selected from fibronectin and vitronectin and typically is functional for binding to collagen.

To be mentioned also are agents, for example antibody products, which comprise domains which recognize collagen, particularly collagen I and/or collagen III, and one or both of fibronectin and vitronectin. The agents typically bind to such domains.

As another aspect, there are disclosed agents which comprise:
a first portion capable of binding to an intravascular GPVI-binding domain; and
a second portion which is capable of binding to a site other than a GPVI-binding domain and/or comprises a therapeutic or diagnostic moiety.

In embodiments the second portion is capable of binding to a binding site for a platelet-bound ligand other than GPVI, for example GPIIb/IIIa, GPIa/IIa, GPIV, GPIc/IIa, GPIb/IX. Also to be mentioned are embodiments in which the second portion confers inhibitor activity against platelet adhesion, platelet aggregation or coagulation. The second portion may comprise an imaging agent, for example a radio-imaging agent, e.g. Tc^{99m}. The agent may comprise a plurality of said second portions. The first portion may capable of binding to one, two or three proteins selected from collagen, e.g. collagen I and collagen III, vitronectin and fibronectin. The first portion may capable of binding to a plurality of intravascular GPVI-binding domains, each domain optionally being of a different type, for example it may be capable of binding to collagen and to one or both of vitronectin and fibronectin. The first portion may be a distributed portion having spaced apart regions having different binding functions; to look at this concept another way, the agent may contain a plurality of first portions.

It will be understood that the disclosure includes agents which are functional to bind to collagen and to one or both of vitronectin and fibronectin, and which further have another therapeutic or diagnostic function.

As another aspect of the disclosure, there may be mentioned agents capable of interfering with a plurality of platelet-blood vessel interactions, of which at least one interaction involves GPVI; the interactions may include interaction with one or both of vitronectin and fibronectin.

Included are pharmaceutical or, as applicable, diagnostic formulations comprising an agent of the disclosure.

The specification further discloses implantable, optionally intravascular, devices which in use expose or release a direct or indirect GPVI inhibitor or any agent disclosed herein, for example one which inhibits interaction between GPVI and a protein selected from collagen, vitronectin and fibronectin, and combinations thereof. In embodiments, the devices have a coating or impregnate comprising such an agent. Exemplary devices are selected from a stent, a vascular catheter, a vascular shunt, a balloon catheter, an autologous venous/arterial graft, a prosthetic venous/arterial graft and a guidewire.

It will be understood that there is included in the disclosure a method of inhibiting or preventing restenosis, thrombosis, atherogenesis, atheroprogression, atherosclerosis, and/or vascular inflammation in a patient, said method comprising implanting in said patient an intravascular device comprising a direct or indirect GPVI inhibitor adapted to be exposed and/or released when the device is implanted.

A particular subject matter of the disclosure is methods for the primary prophylaxis of cardiovascular disease, comprising administering an effective amount of an agent capable of interfering with interaction between GPVI and one or both of vitronectin and/or fibronectin, or of an agent comprising an amino acid sequence derived from an extracellular domain of GPVI.

In embodiments, there are provided methods for treating a patient suffering from, or at risk of suffering from, a disorder characterised by a pathological interaction between GPVI and fibronectin and/or vitronectin, comprising administering an effective amount of an agent which inhibits interaction between GPVI and a protein selected from the group consisting of fibronectin, vitronectin and combinations thereof. The disorder may be initiation of atherosclerosis.

Additionally to be mentioned is a method for secondary prophylaxis of a patient who has suffered, or is at risk or suspicion of having suffered, an atherosclerotic event, comprising administering to the patient an effective amount of an agent which inhibits interaction between GPVI and a protein, e.g. two or three proteins, selected from the group consisting of collagen, fibronectin, vitronectin and combinations thereof. In embodiments, the patient suffered, or is at risk or suspicion of having suffered, a disorder selected from the group consisting of thrombosis, myocardial infarction, stroke, transient ischemic attack, occlusive peripheral vascular disease, occlusion of a peripheral artery and complications thereof.

Another method disclosed herein is one of treating a patient who has suffered a cardiovascular event comprising administration to the patient of an effective amount of a direct or indirect GPVI inhibitor for at least three months after said cardiovascular event. For example, the GPVI inhibitor may inhibit the interaction between GPVI and a protein selected from the group consisting of fibronectin, vitronectin, collagen and combinations thereof. The GPVI inhibitor may be administered for a period of up to six months after said cardiovascular event. The cardiovascular event may comprise rupture of atherosclerotic plaque. It may be thrombosis or myocardial infarction.

The specification discloses also the local administration to an actual or suspected site of an atherosclerotic disorder of an inhibitor of GPVI binding. Such administration is useful in the treatment of a patient suffering from, or suspected to be suffering from, an atherosclerotic disorder, e.g. an atherosclerotic plaque which may be ruptured. The administration may be via a catheter.

Amongst other methods disclosed herein are:
a method for treating a patient having, or identified as having, at least one or a combination of risk factors for the formation of atherosclerotic lesions, comprising administering an effective amount of an agent which inhibits interaction between GPVI and a protein selected from the group consisting of fibronectin, vitronectin and combinations thereof;
a method of treating a patient who has, or has been identified as having, one or more risk factors associated with developing atherosclerosis, said patient having not yet developed advanced atherosclerotic plaques, wherein said patient is considered to have a risk score of 45 or greater according to the PROCAM study, said method comprising administering an agent which inhibits binding of GPVI to a molecule selected from vitronectin, fibronectin and combinations thereof;
a method for preventing or retarding atherosclerotic cardiovascular disease in a patient who is not displaying clinical symptoms of atherosclerosis, the method comprising administering to the patient an agent which inhibits binding of GPVI to a molecule selected from vitronectin, fibronectin and combinations, for example wherein the patient has a 10-year risk of fatal cardiovascular disease according to the SCORE project of at least 3%;
a method for the primary prevention of atherosclerotic cardiovascular disease in a patient, the method comprising administering to the patient an agent which inhibits binding of GPVI to a molecule selected from vitronectin, fibronectin and combinations thereof;
a method for treating patient having, or identified as having, a marker of vascular inflammation, comprising administering to the patient an agent which inhibits binding of GPVI to a molecule selected from vitronectin, fibronectin and combinations thereof.
a method for treating atherosclerosis in a patient, comprising administering to the patient an agent capable of binding to a plurality of GPVI-binding sites at a frequency of once a day or less, e.g. of at most once every 48 hours.

Further provided is an agent which inhibits GPVI interaction with fibronectin, for example the agent is capable of inhibiting GPVI binding to, or association with fibronectin. Included in the disclosure are agents that bind to fibronectin, particularly at a GPVI binding site; the agent may bind to GPVI, for example at a fibronectin binding site.

Further provided by the invention is an agent which inhibits GPVI interaction with vitronectin, for example the agent is capable of inhibiting GPVI binding to, or association with vitronectin. Included in the disclosure are agents that bind to vitronectin, particularly at a GPVI binding site; the agent may bind to GPVI, for example at a vitronectin binding site.

Included are agents which are capable of inhibiting GPVI binding to, or association with, both vitronectin and fibronectin.

Agents of the disclosure may be capable of inhibiting GPVI binding to, or association with, one or more, or a combination of, collagen, vitronectin and fibronectin.

The disclosure also includes methods of treatment which use not a single entity having plural functions but plural entities, each entity having a different function or combination of functions from the other(s). The functions of the entities may include, or be, inhibition of interactions of blood components and/or the vasculature, for example interactions involving any one or more of platelets, monocytes, matrix proteins e.g. collagen, and adhesion proteins e.g. fibronectin and/or vitronectin; such interactions are described in more detail hereinafter in various contexts and all described interactions are applicable to entity combinations. As entities may be mentioned antibodies and low molecular weight molecules.

The disclosure also includes more generally combination products and therapies involving a product of the disclosure, for example therapy in combination with another anti-thrombotic drug, particularly one have a mode of action not involving GPVI.

Further aspects and embodiments of the disclosure are set forth in the following description and claims.

### BRIEF DESCRIPTION OF THE DRAWINGS

For details of the materials and methods used in the following results, see WO 03/104282.

FIG. 1 Platelet adhesion and aggregation following vascular injury of the common carotid artery in C57BL6/J mice in vivo. (a) Scanning electron micrographs of carotid arteries prior to (left panels) and 2 hrs after (right panels) vascular injury. Endothelial denudation induces platelet adhesion and aggregation, resulting in the formation of a platelet-rich (lower left) thrombus. (b) Platelet-endothelial cell interactions 5 min after vascular injury were investigated by in vivo fluorescence microscopy of the common carotid artery in situ (black columns). Animals without vascular injury served as controls (open columns). The left and right panels summarize transient and firm platelet adhesion, respectively, of eight experiments per group. Platelets were classified according to their interaction with the endothelial cell lining as described²⁴ and are given per mm² of vessel surface. Mean±s.e.m., asterisk indicates significant difference compared to control, P<0.05. (c) Platelet aggregation following vascular injury was determined by fluorescence microscopy in vivo (black columns). Animals without vascular injury served as controls (open columns). Mean±s.e.m., n=8 each group, asterisk indicates significant difference compared to wild type mice, P<0.05. The microphotographs (right) show representative in vivo fluorescence microscopy images in control animals (upper panel) or following vascular injury (lower panel). White arrows indicate adherent platelets.

FIG. 2 Inhibition of GPVI abrogates platelet adhesion and aggregation after vascular injury. (a) Platelet adhesion following vascular injury was determined by intravital videofluorescence microscopy. Fluorescent platelets were preincubated with 50 μg/ml anti-GPVI (JAQ1) Fab fragments or control rat IgG. Platelets without mAb preincubation served as control. The left and right panels summarize transient and firm platelet adhesion, respectively. Mean±s.e.m., n=8 each group, asterisk indicates significant difference compared to control, P<0.05. (b) illustrates the percentage of platelets establishing irreversible adhesion after initial tethering/slow surface translocation is. (c) Platelet aggregation following vascular injury in vivo. Aggregation of platelets preincubated with tyrodes, irrelevant rat IgG, or anti-GPVI Fab (JAQ1) was assessed by fluorescence microscopy as described. Mean±s.e.m., n=8 each group, asterisk indicates significant difference compared to control, P<0.05. (d) The photomicrographs show representative in vivo fluorescence microscopy images illustrating platelet adhesion in the absence or presence of anti-GPVI Fab (JAQ1) or control IgG.

FIG. 3 Platelet adhesion following endothelial denudation in GPVI-deficient mice. (a) JAQ1-treated mice lack GPVI. Upperpanels: Platelets from mice pretreated with irrelevant control IgG (left) or anti-GPVI (JAQ1) (right) were incubated with FITC-labeled JAQ1 and PE-labeled anti-mouse CD41 for 10 min at room temperature and directly analyzed on a FACScan™. A representative dot blot of 3 mice per group is presented. Lower panel: Whole platelet lysates from three control IgG or JAQ1-treated mice were separated by SDS-PAGE under non-reducing conditions and immunoblotted with FITC-labeled JAQ1, followed by incubation with HRP-labeled rabbit-anti-FITC mAb. (b) Scanning electron micrographs of carotid arteries 2 hrs after vascular injury in control animals (upper panels) or GPVI-depleted mice (lower panels). Endothelial denudation induced platelet adhesion and platelet aggregation in control animals. In contrast, only very few platelets attached along the damaged vessel wall in GPVI-depleted mice. Subendothelial collagen fibers are visible along the denuded area. (c) Platelet tethering and firm platelet adhesion, (d) transition from initial tethering to stable arrest (percentage of tethered platelets), and (e) platelet aggregation following vascular injury of the carotid artery was determined in GPVI-deficient (JAQ1-pretreated mice) or control IgG-pretreated mice (for details see Materials and Methods). The panels summarize platelet adhesion (transient and firm) and platelet aggregation in eight experiments per group. Mean±s.e.m., asterisk indicates significant difference compared to control IgG, P<0.05. (f) The photomicrographs show representative in vivo fluorescence microscopy images illustrating platelet adhesion in GPVI-deficient (JAQ1) and control IgG-treated mice.

FIG. 4 Platelet adhesion to the surface of collagen coated glass coverslips under physiological flow conditions was assessed ex vivo. Left panel: Platelets from mice pretreated with irrelevant control IgG immunoadhesin (control) (left) or anti-GPVI immunoadhesin (Fc-GP VI-nt) (right) were investigated for adhesion under physiological flow conditions. The number of platelets was assessed by FACS counting of the washed coverslips at the end of each experiment. Platelet tethering as the first step of platelet adhesion was assessed after 30 seconds and firm platelet adhesion after 5 min under flow conditions. The panels summarize transient and firm platelet adhesion in eight experiments per group. Mean±s.e.m., asterisk indicates significant difference compared to control IgG, P<0.05.

FIG. 5 Interaction of Fc-GP VI-nt with collagen was monitored in an ELISA based assay. Adhesion of the immunoadhesin—a GPVI-Fc fusion protein comprising a Gly-Gly-Arg linker and the FC part of an IgG to collagen coated plates with increasing concentrations of Fc-GP VI-nt (0.5 μg to 10 μg) was investigated. The binding is visualised with a secondary antibody labelled with peroxidase directed to the Fc part of Fc-GP VI-nt. Peroxidase is finally detected by ELISA. In this representative experiment binding of Fc-GP VI-nt to collagen was monitored with sufficient affinity, which reached saturation at μg concentrations.

FIG. 6 Interaction of the—a GPVI-Fc fusion protein comprising a Gly-Gly-Arg linker with collagen and the possibility to screen for GP VI inhibitors was demonstrated with the inhibitory anti mouse GP VI antibody JAQ1. Adhesion of the immunoadhesin Fc-GP VI-nt (2 μg/well) to collagen coated ELISA plates is shown to be specific: the empty immunoadhesin Fc-nt did not show any binding. Thus, this provides an ELISA based assay for the screening against GP VI inhibitors with the upscale potential to high-throughput capacities.

FIG. 7 Amino acid sequence of—a GPVI-Fc fusion protein comprising a Gly-Gly-Arg linker: SEQ ID No: 1.

FIG. 8 DNA-Sequence of—a GPVI-Fc fusion protein comprising a Gly-Gly-Arg linker: SEQ ID No. 2. Bases 1 to 807 encode the extracellular domain of GP VI. Bases 817 to 1515 encode the Fc part of the IgG.

FIG. 9 Characterization of—a GPVI-Fc fusion protein comprising a Gly-Gly-Arg linker. (a) upper panel: Fc-GPVI-nt and control Fc lacking the extracellular GPVI domain were used for SDS-PAGE under reducing conditions. Coomassie blue stain (left) and immunoblotting with peroxidase-conjugated goat anti-human Fc antibody (right) identified Fc-GPVI-nt with a molecular mass of ˜80 kDa. Middle panel: Immunoblotting of Fc, Fc-GPVI-nt, or human platelets using the anti-GPVI monoclonal antibody 5C4. 5C4 detected both adenovirally expressed Fc-GPVI-nt fusion protein and platelet GPVI, but not the control Fc. Lower panel: Molecular mass under reducing (right) and non-reducing (left) conditions. While the molecular mass of Fc-GPVI-nt was approximately 80 kDa under reducing conditions, the complete nt with ˜160 kDa protein was identified under non-reducing conditions. (b-d) Characterization of Fc-GPVI-nt collagen interactions. (b) Binding assays using different concentrations of soluble Fc-GPVI-nt and immobilized collagen (10_g/ml) were performed to define Fc-GPVI-nt-collagen interactions. Bound Fc-GPVI-nt was detected by anti-Fc mAb antibody (dilution 1:10.000) and is given relative to the binding observed at 10_g/ml Fc-GPVI-nt. Fc-GPVI-nt binds to collagen in a saturable manner. Mean±s.e.m., n=6 each Fc-GPVI-nt concentration, asterisk indicates significant difference compared to 0_g/ml Fc-GPVI-nt, P<0.05. (c, left panel) shows binding of Fc-GPVI-nt (20 μg/ml) to various substrates. Binding of Fc-GPVI-nt to BSA (10_g/ml) or vWF (10_g/ml) is given as percentage of GPVI-dimer-binding to immobilized collagen. Binding of Fc-GPVI-nt did not occur to BSA or vWF, supporting the specificity of Fc-GPVI-nt binding. Mean±s.e.m., asterisk indicates significant difference compared to collagen, P<0.05. (c, right panel) illustrates binding of Fc-GPVI-nt (20 μg/ml) or Fc (20 μg/ml) to immobilized collagen (10 μg/ml). Bound Fc-GPVI-nt or Fc was detected by anti-Fc mAb antibody (dilution 1:10.000) and is given relative to the binding observed with Fc-GPVI-nt. Only Fc-GPVI-nt, but not Fc or anti-Fc mAb binds to immobilized collagen. Mean±s.e.m., n=8 each group, asterisk indicates significant difference compared to Fc-GPVI-nt binding, P<0.05. (d) Fc-GPVI-nt (20 μg/ml) was preincubated for 10 min with different concentrations of soluble collagen. After incubation the plates were washed and Fc-GPVI-nt binding was detected by peroxidase-conjugated goat anti-human IgG antibody (dilution 1:10.000). Fc-GPVI-nt binding is given relative to the binding observed in the absence of soluble collagen. Soluble collagen inhibits GPVI-Fc-dimer-dimer binding to immobilized collagen in a dose-dependent manner. Mean±s.e.m., n=3 each collagen concentration, asterisk indicates significant difference compared to 0_g/ml collagen, P<0.05. (e) The difference of the binding affinity between the monomeric form of the GPVI-Fc fusion protein and Fc-GPVI-nt was assessed in direct comparison. The binding of the monomer and dimer was assessed on collagen type 1 coated ELISA plates. Increasing concentrations of the GPVI fusion proteins bond to collagen in a saturable manner. Here a Lineweaver Burke plot is demonstrated for affinity assessment (e). The affinity of the monomeric GPVI fusion protein was about 10 times lower compared to equimolar concentrations of the dimeric form Fc-GPVI-nt.

FIG. 10 Fc-GPVI-nt inhibits CD 62 P activation on human platelets as a parameter of release of intracellular transmitter substances from alpha granules by increasing doses of collagen. Human platelets were isolated from whole blood and incubated with anti-CD 62 antibodies labelled with PE. Fluorescence was determined in a Becton Dickenson FACS device. Representative histograms are shown. Increasing concentrations of collagen from 0 to 10 μg/ml induced a shift of fluorescence in the presence of the control Fc protein (100 μg/ml; blue line). In the presence of Fc-GPVI-nt (100 μg/ml; red line), the shift of fluorescence and hence CD 62 P activation was markedly inhibited.

FIG. 11 Specific inhibition of collagen-mediated platelet aggregation and release of endogenous transmitters from dense and alpha granules by Fc-GPVI-nt. (a) Human platelets were incubated with control Fc (80 μg/ml) or Fc-GPVI-nt (80 μg/ml). Aggregation of platelets was induced with collagen (1 μg/ml) or ADP (5 μM) or TRAP (10 μM) and aggregation was determined in an aggregometer under stirring conditions. Triplet measurements from n=5 different blood donors were carried out. The means±s.e.m are given in % aggregation of the control aggregation without fusion proteins. (b) ATP release was measured simultaneously in the same probes after incubation with control Fc (80 μg/ml) or Fc-GPVI-nt (80 μg/ml). The amount of ATP release is given in % of controls without fusion protein. (c) PDGF release was determined in human platelets with an ELISA system specific for human PDGF under basal conditions and after collagen (20 μg/ml) stimulation. Preincubation with control Fc had no significant effect on PDGF release from collagen-stimulated platelets, whereas Fc-GPVI-nt (100 μg/ml) reduced the PDGF release significantly. Inhibition of PDGF release did not occur in unstimulated platelets.

FIG. 12 Fc-GPVI-nt has no significant effect on bleeding time in human blood ex vivo. Bleeding time in human blood was measured ex vivo after ADP/collagen stimulation and epinephrine/collagen stimulation in a PFA-100 device. Fc-GPVI-nt (5 and 20 μg/ml) and Fc (5 and 20 μg/ml) did not prolong bleeding time whereas ReoPro^{R} in a therapeutically relevant concentration (5 μg/ml) maximally prolonged bleeding time under both conditions. The means±s.e.m. from n=4 blood donors with triplet measurements are summarized.

FIG. 13. Fc-GPVI-nt inhibits platelet adhesion to immobilized collagen under flow conditions. Human platelets (2×10⁸ cells/ml) were isolated from whole blood. Plates were coated with immobilized collagen (10_g/ml) or vWF (10_g/ml). Platelet adhesion to the coated plates was determined in a parallel plate flow chamber in the presence of Fc-GPVI-nt or Fc lacking the extracellular GPVI domain (200 μg/ml). Inhibition of platelet adhesion by Fc-GPVI-nt is given in % of control (Fc control). Fc-GPVI-nt significantly attenuated platelet adhesion on immobilized collagen at shear rates of 500 sec⁻¹ and 1000 sec⁻¹, respectively. In contrast, Fc-GPVI-nt did not affect platelet adhesion on immobilized vWF. Mean±s.e.m., n=4 each group, asterisk indicates significant difference compared to control Fc, P<0.05. The lower panels show representative microscopic images.

FIG. 14 Fc-GPVI-nt has favourable pharmacokinetics with a prolonged plasma half life after intraperitoneal injection in mice in vivo. Blood concentrations of Fc-GPVI-nt were determined with specific anti-Fc antibodies and ELISA. (a) Single intraperitoneal injection of Fc-GPVI-nt (4 μg/g) led to rapid peak blood concentrations of Fc-GPVI-nt after ˜24 h with slow decline of Fc-GPVI-nt blood concentrations. The means±s.e.m. from 10 animals are demonstrated. (b) Repeated intraperitoneal applications (10 μg/g; twice weekly) leads to continuous accumulation of Fc-GPVI-nt in mice in vivo over 28 days. The means±s.e.m. from 6 animals are demonstrated. (c) Intravenous single dose injection of 30 μg Fc-GPVI-nt (1 μg/g body weight); 60 μg (2 μg/g body weight) and 100 μg Fc-GPVI-nt (3 μg/g body weight) per mouse led to a dose-dependent increase of immunoadhesin plasma concentration. The plasma concentration in the two higher doses in these mice in vivo reached prolonged elevated levels from 5 to 60 minutes and after 24 hours, sufficient for effective collagen scavenging and therefore effective inhibition of GPVI receptor activation on platelets. The means±s.e.m. from 5 animals are demonstrated.

FIG. 15 Effects of Fc-GPVI-nt on platelet adhesion and aggregation in vivo. (a) Mice (n=6 per group) were treated with 2 mg/kg or 4 mg/kg Fc-GPVI-nt Iv. Intergrilin (0,2 mg/kg)-treated mice served as positive controls (n=8). Bleeding times were determined as described (see “materials and methods”). The Fc-GPVI-nt fusion protein did not increase tall bleeding times compared to control animals. In intergrilin-treated mice tall bleeding time was massively prolonged. **P<0.05 vs. control. (b) Inhibition of GPVI abrogates platelet adhesion and aggregation after vascular injury. Platelet adhesion following vascular injury was determined by intravital video fluorescence microscopy. Mice were pretreated with 1 or 2 mg/kg Fc-GPVI-nt or equimolar amounts of control Fc. The left and right panels summarize platelet tethering and firm platelet adhesion, respectively. Mean±s.e.m., n=5 each group, asterisk indicates significant difference compared to Fc, P<0.05. (c) Effects of Fc-GPVI-nt on thrombus formation following vascular injury in vivo. The number of platelet thrombi (right) and the total thrombus area (left) were assessed by fluorescence microscopy as described. Mean±s.e.m., n=5 each group, asterisk indicates significant difference compared to Fc, P<0.05. (d) The photomicrographs show representative in vivo fluorescence microscopy images illustrating platelet adhesion in the absence or presence of 1 or 2 mg/kg Fc-GPVI-nt or control Fc. Bars represent 50_m. (e) Scanning electron micrographs of carotid arteries 1 hr after vascular injury in Fc- or Fc-GPVI-nt treated animals. Endothelial denudation induced platelet adhesion and platelet aggregation in Fc-treated mice. In contrast, only very few platelets attached along the damaged vessel wall in Fc-GPVI-nt-treated mice. Subendothelial collagen fibers are visible along the denuded area. Bars represent 10_m (f) Fc-GPVI-nt specifically binds to the subendothelium of carotid arteries. The binding of Fc-GPVI-nt to the subendothelium was determined on carotid sections, stained with peroxidase-conjugated goat anti-human IgG antibody. Carotid arteries obtained from Fc-treated mice served as controls. Fc-GPVI-nt but not Fc control protein was detected at the subendothelium, as indicated by the brown staining. Original magnification: 100-fold.

FIG. 16 Fc-GPVI-nt significantly attenuates atheroprogression in apo e −/− knockout mice in vivo. Apo e −/− mice were treated with Fc-GPVI-nt (4 μg/g) or control Fc (4 μg/g) intraperitoneally for 4 weeks twice weekly. Atheroprogression was investigated post mortem after sudan red staining of the large vessels to visualise atheroma and plaque formation. In control animals extensive plaque formation of carotid artery preparations was indicated by the red colour in particular in the branching region. In Fc-GPVI-nt treated animals atherosclerosis was almost completely abolished in carotid arteries of apo e −/− mice. Representative macroscopic whole vascular preparations of the carotid arteries of an apo e −/− mouse after 4 weeks treatment with Fc-GPVI-nt (left side) and of an apo e −/− mouse after 4 weeks treatment with the control Fc protein (right side) are shown.

FIG. 17 Freshly isolated platelets from patients suffering from diabetes mellitus show reduced expression of the fibrinogen receptor (CD61, top) and increased expression of the Fc receptor (CD32, middle) and therefore increased expression of GPVI. The correlation between CD32 expression and GPVI expression (detected by the specific monoclonal antibody 4C9) is shown on human platelets (bottom). Human platelets were isolated from whole blood from patients suffering from diabetes and incubated with fluorescent anti-CD61 and anti CD32 antibodies or FITC labelled 4C9 antibodies. Fluorescence was determined in a Becton Dickenson FACScalibur device. The means+/−s.e.m. from n=111 diabetic patients and from n=363 patients without diabetes are summarized. Correlation of CD32 fluorescence and 4C9 fluorescence was calculated with the correlation coefficient r=0.516.

FIG. 18 Amino acid sequence of a monomeric fusion protein based on Fc-GPVI-nt.

FIG. 19

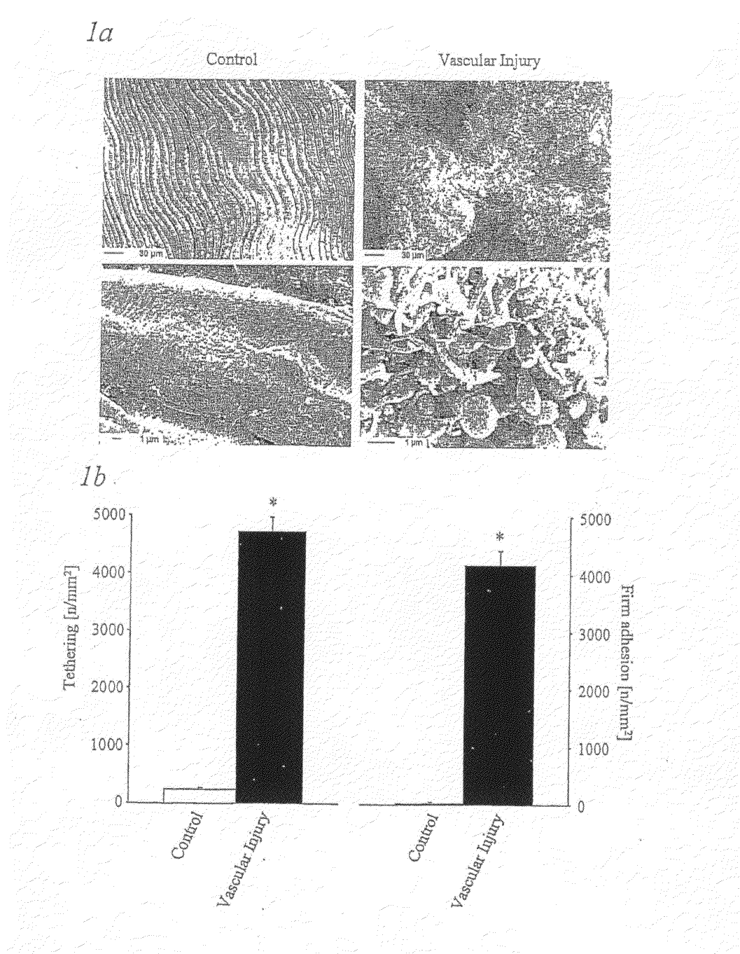

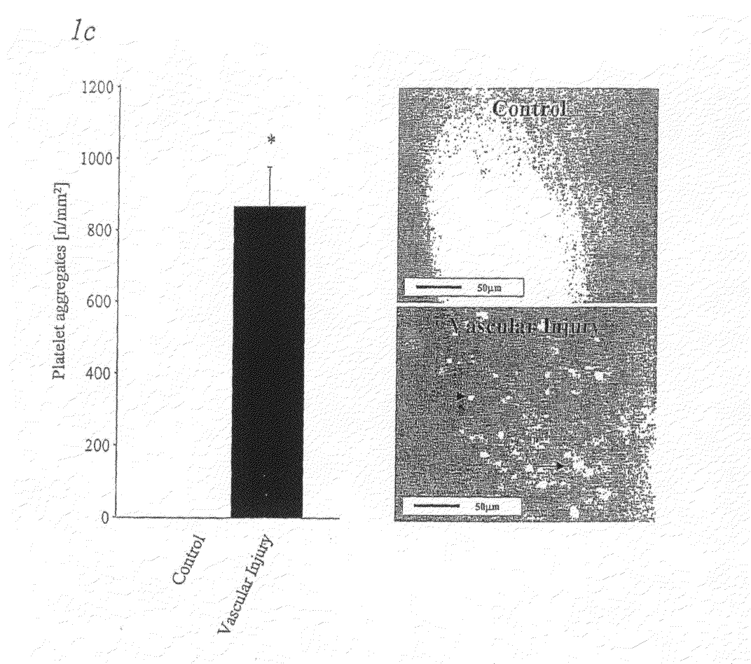

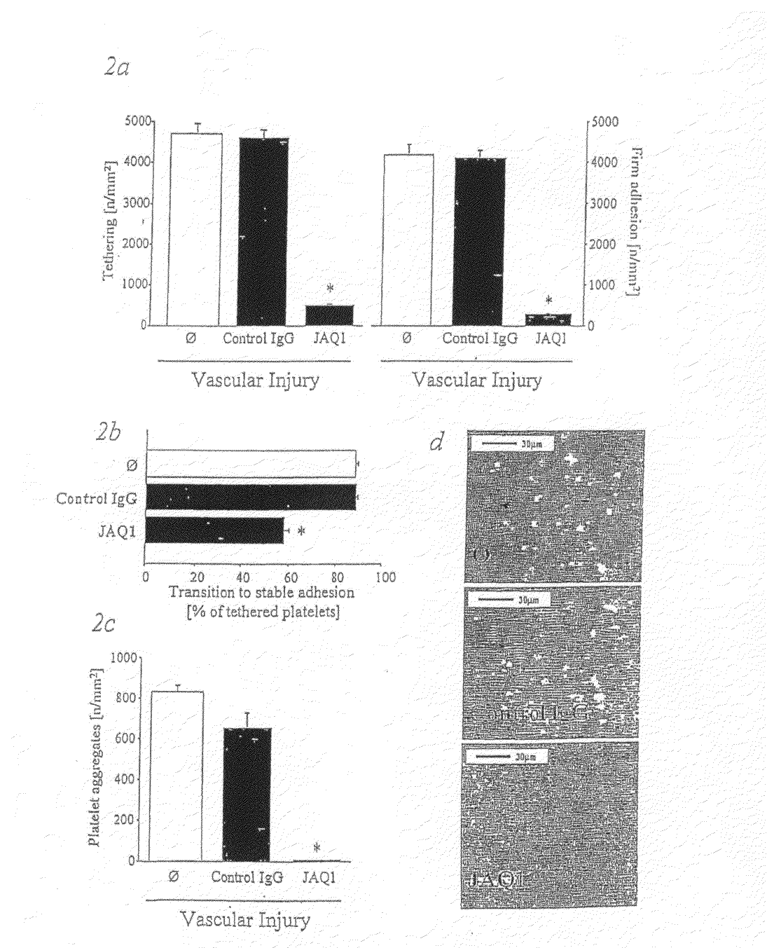

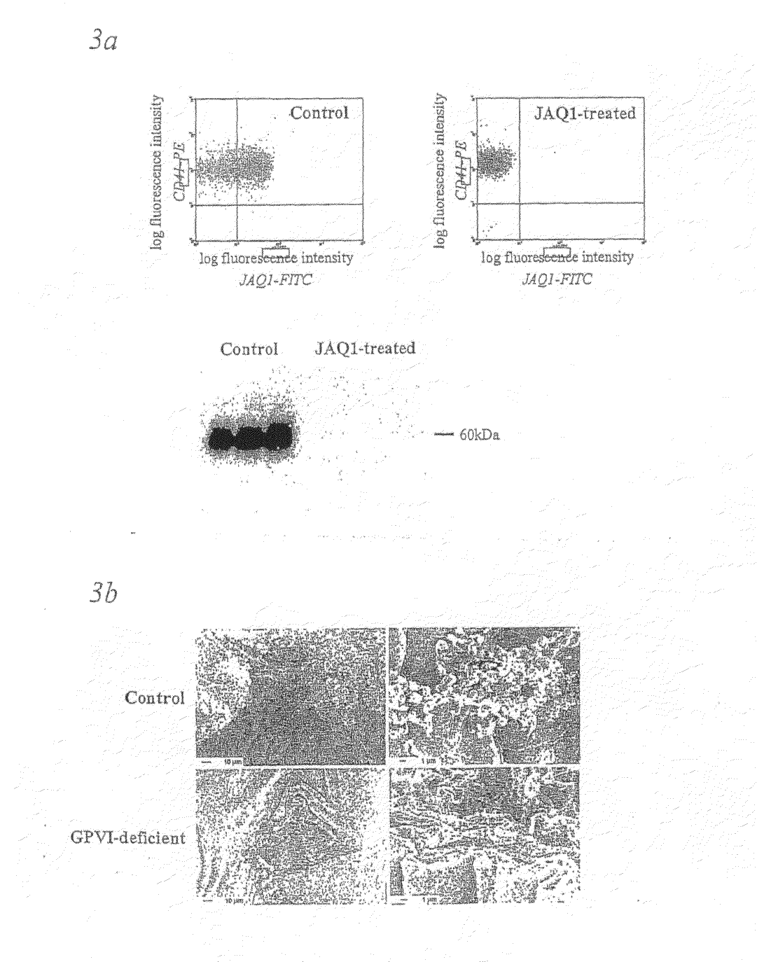

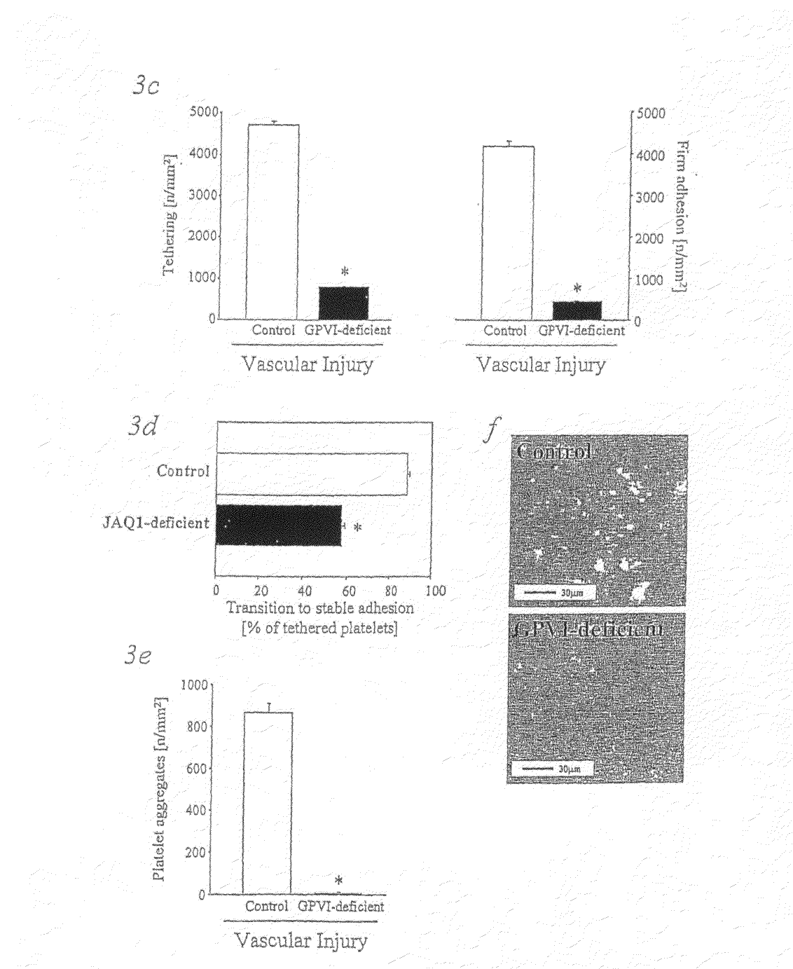

Identification of endothelial GPVI ligands. a, Isolated human platelets were perfused at a wall shear rate of 2000 s-1 (10 min) over confluent monolayer of HUVEC pre-activated for 16 hours with TNF-α (50 ng/ml) and INF-γ (20 μg/ml). As indicated, HUVEC were pre-treated with soluble GPVI-Fc or control Fc for 30 min before initiation of perfusion. Mean±standard deviation of 5 experiments. * indicates significant differences compared with control (p<0.01). b, HUVEC were activated with TNF-α/INF-γ and incubated with soluble GPVI-Fc or control Fc. After addition of secondary FITC-conjugated anti-Fc mAb, HUVEC were detached from the culture wells and analysed by flow cytometry. Mean±standard deviation of 8 experiments. c, Confluent monolayers of HUVEC were left untreated or activated with TNF-α/INF-γ. HUVEC were stained with FITC-conjugated anti-ICAM-1, anti-vWF, anti-CD62P, LM609, anti-CD61, anti-CD51, anti-Fn, or IgG control mAb and binding of antibodies was analysed by flow cytometry. Mean±standard deviation (n=3). d, Binding assays using soluble GPVI-Fc (10 μg/ml) or control Fc (10 μg/ml) to immobilized bovine collagen type 1 (1 μg/ml), human Vn (1 μg/ml), human Fn (1 μg/ml), and human vWF (1 μg/ml) was performed as described¹². Bound GPVI-Fc was detected by anti-Fc mAb and is given relative to the binding over non-specific Fc control. Mean±standard deviation, 4 to 8 experiments for each GPVI-Fc concentration. e, Soluble GPVI-Fc was pre-incubated with blocking Fab fragment of anti-GPVI mAb 5C4 and binding experiments to immobilized collagen, Vn, and Fn were performed as described under d.

FIG. 20

Functional characterization of GPVI for platelet adhesion. a, Plates coated with collagen, Fn, Vn, or vWF were perfused with human platelets after preincubation of ligands with GPVI-Fc or Fc. Percentage inhibition of platelet adhesion by GPVI-Fc is given. In addition, the effect of 1 mM EDTA on GPVI-dependent inhibition of platelet adhesion to immobilized ligands was evaluated. Mean and standard deviation (n=4 to 5). b, Adhesion of stable GPVI-expressing (CHO-GPVI) or non-transfected CHO cells to immobilized Fn, Vn, collagen, or non-coated plastic culture plates. Mean±SD (n=4). The panels in the right row show representative microscopic images. c, Platelets from apoE^{-/-} mice (n=5 per group) were treated with vehicle, control IgG (50 μg/ml), or 5C4 Fab (50 μg/ml). Platelet adhesion at the carotid artery was evaluated by intravital fluorescence microscopy³. Mean±SD (n=5). d, The binding of GPVI-Fc or control Fc to the endothelial monolayer on diseased carotid arteries was determined on carotid sections stained with peroxidase-conjugated goat anti-human IgG antibody (original magnification: 100-fold). e, Fixed cryostat sections of carotid arteries were incubated with hGPVI-Fc, anti-Fn mAb (Santa Cruz) and thereafter fluorescently labelled for detection of Fn (green), GPVI (red), and nuclei (blue). Confocal microscopy (×100 objective) shows individual details of Fn-(left), GPVI-stalning (middle), and the overlay (right) with yellow colour indicating co-localization.

FIG. 21 in vivo analysis of GPVI inhibition on vascular remodelling, a, b, ApoE^{-/-} mice were treated with irrelevant rat IgG or ant-GPVI JAQ1 mAb for 4 weeks. Atherosclerotic lesion formation was assessed in the aortic arch and the right coronary artery bifurcation. The extension of fatty streaks (μm) was quantified by Sudan III staining. Data represent mean values with SEM (4-6 experiments per group. Depicted are representative photomicrographs of oil red staining of a carotid artery and an aortic arch from control- and JAQ1-treated animals.

FIG. 22 Double balloon catheter used for local delivery of soluble GPVI.

FIG. 23 To assess the efficacy of local balloon-based delivery of GPVI, immunostaining of serial cross sections of carotid arteries were performed. Carotid cross sections were stained with a specific monoclonal antibody (5C4) directed against GPVI. Substantial positive staining for GPVI of injured carotid segments, but not of intact carotid arteries was found.

FIG. 24 Using a modified double-balloon drug-delivery catheter sGPVI (10 μg/ml) or control protein (Fc (10 μg/ml) was delivered to site of injury, and the respective carotid segments were incubated for 5 min with SsGPVI or the control. Thereafter, thrombus formation was assessed by direct microscopy. sGPVI treatment of carotid lesions resulted in an approximately 80% reduction of thrombus formation (10.xx vs. 2.yy, p<0.0×).

FIG. 25 Comparison of the effect on platelet aggregation of GPVI-Fc fusion protein comprising a triple alanine linker and a GPVI-Fc fusion protein comprising a Gly-Gly-Arg linker.

FIG. 26 Comparison of the effect on ATP release of GPVI-Fc fusion protein comprising a triple alanine linker and a GPVI-Fc fusion protein comprising a Gly-Gly-Arg linker.

FIG. 27 Comparison of the effect on thrombus formation of GPVI-Fc fusion protein comprising a triple alanine linker and a GPVI-Fc fusion protein comprising a Gly-Gly-Arg linker.

### DETAILED DESCRIPTION

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

The extent of protection includes counterfeit or fraudulent products which contain or purport to contain a compound of the invention irrespective of whether they do in fact contain such a compound and irrespective of whether any such compound is contained in a therapeutically effective amount. Included in the scope of protection therefore are packages which include a description or instructions which indicate that the package contains a species or pharmaceutical formulation of the invention and a product which is or comprises, or purports to be or comprise, such a formulation or species.

### DEFINITIONS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Definitions and additional information known to one of skill in the art in immunology can be found, for example, in Fundamental Immunology, W. E. Paul, ed., fourth edition, Lippincott-Raven Publishers, 1999.

Antibody fragment (fragment with specific antigen binding): It has been shown that fragments of a whole antibody can perform the function of binding antigens. Various fragments of antibodies have been defined, including Fab, (Fab′)2, Fv, single domain antibodies and single-chain Fv (scFv). These antibody fragments are defined as follows: (1) Fab, the fragment that contains a monovalent antigen-binding fragment of an antibody molecule produced by digestion of whole antibody with the enzyme papain to yield an intact light chain and a portion of one heavy chain or equivalently by genetic engineering. The Fab fragment therefore contains VL, VH, CL and CH1 domains; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′) 2, the fragment of the antibody obtained by treating whole antibody with the enzyme pepsin without subsequent reduction or equivalently by genetic engineering; (4) F(Ab′) 2, a dimer of two FAb′ fragments held together by disulfide bonds (5) Fv, a genetically engineered fragment containing the variable region of the light chain and the variable region of the heavy chain expressed as two chains; (6) single domain antibodies; antibodies whose complementary determining regions are part of a single domain polypeptide; and (7) single chain antibody (“scFV”), a genetically engineered molecule containing the variable region of the light chain, the variable region of the heavy chain wherein the VH domain and the VL domain are linked by a peptide linker which allows the two domains to associate to form an antigen binding site (Bird et al, Science, 242, 423-426, 1988; Huston et al, PNAS USA, 85, 5879-5883, 1988); (8) the dAb fragment (Ward, E. S. et al., Nature 341, 544-546 (1989)) which consists of a VH domain; (9) bispecific single chain Fv dimers (PCT/US92/09965) and (10) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al, Nature Biotech, 14, 1239-1245, 1996). Minibodies comprising ascFv joined to a CH3 domain may also be made (S. Hu et al, Cancer Res., 56, 3055-3061, 1996). Methods of making these fragments are routine in the art. Other antibody fragments are also considered.

dAB (domain antibodies) are the smallest functional binding units of antibodies, corresponding to the variable regions of either the heavy (VH) or light (VL) chains of human antibodies. Domain Antibodies have a molecular weight of approximately 13 kDa, or less than one-tenth the size of a full antibody.

Domain antibodies may include dAbs which bind to two therapeutic targets. These include: IgG-like molecules; PEGylated fusion proteins; and anti-serum albumin fusion proteins. In the IgG-like antibody, two variable domains bind to two therapeutic targets on each arm of the IgG.

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against GPVI, then a library can be made where the other arm is varied and an antibody of appropriate specificity selected. Bispecific whole antibodies may be made by knobs-into-holes engineering (J. B. B. Ridgeway et al, Protein Eng., 9,616-621, 1996). or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

Single domain antibodies: Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one embodiment of the invention, a single domain antibody as used herein is a naturally occurring single domain antibody known as heavy chain antibody devoid of light chains. Such single domain antibodies are disclosed in WO 9404678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

VHHs, according to the present disclosure, and as known to the skilled addressee, are heavy chain variable domains derived from immunoglobulins naturally devoid of light chains such as those derived from Camelidae as described in WO9404678 (and referred to hereinafter as VHH domains or Nanobodies®). VHH molecules are about 10× smaller than IgG molecules.

Complementarity-determining region (CDR): The CDRs are three hypervariable regions within each of the variable light (VL) and variable heavy (VH) regions of an antibody molecule that form the antigen-binding surface that is complementary to the three-dimensional structure of the bound antigen. Proceeding from the N-terminus of a heavy or light chain, these complementarity-determining regions are denoted as “CDR1”, “CDR2”, and “CDR3”, respectively. CDRs are involved in antigen-antibody binding, and the CDR3 comprises a unique region specific for antigen-antibody binding. An antigen-binding site, therefore, may include six CDRs, comprising the CDR regions from each of a heavy and a light chain V region. Alteration of a single amino acid within a CDR region can destroy the affinity of an antibody for a specific antigen (see Abbas et al., Cellular and Molecular Immunology, 4th ed. 143-5, 2000). The locations of the CDRs have been precisely defined, e.g., by Kabat et al., Sequences of Proteins of Immunologic Interest, U.S. Department of Health and Human Services, 1983.

Reference is made to the numbering scheme from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. For purposes of this invention, to assign residue numbers to a candidate antibody amino acid sequence which is not included in the Kabat compendium, one follows the following steps. Generally, the candidate sequence is aligned with any immunoglobulin sequence or any consensus sequence in Kabat. Alignment may be done by hand, or by computer using commonly accepted computer programs; an example of such a program is the Align 2 program discussed in this description. Alignment may be facilitated by using some amino acid residues which are common to most Fab sequences. For example, the light and heavy chains each typically have two cysteines which have the same residue numbers; in VL domain the two cysteines are typically at residue numbers 23 and 88, and in the VH domain the two cysteine residues are typically numbered 22 and 92. Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues (see, e.g. residues 100abcde in FIG. 5). For candidate sequences which, for example, align with a Kabat sequence for residues 34 and 36 but have no residue between them to align with residue 35, the number 35 is simply not assigned to a residue.

CDR and FR residues are also determined according to a structural definition (as in Chothia and Lesk, J. Mol. Biol. 196:901-917 (1987). Where these two methods result in slightly different identifications of a CDR, the structural definition is preferred, but the residues identified by the sequence definition method are considered important FR residues for determination of which framework residues to import into a consensus sequence.

Aptamer: The agent of the present disclosure may be an aptamer. Aptamers have been defined as artificial nucleic acid ligands that can be generated against amino acids, drugs, proteins and other molecules. They are isolated from complex libraries of synthetic nucleic acids by an iterative process of adsorption, recovery and re-amplification.

RNA aptamers are nucleic acid molecules with affinities for specific target molecules. They have been likened to antibodies because of their ligand binding properties. They may be considered as useful agents for a variety of reasons. Specifically, they are soluble in a wide variety of solution conditions and concentrations, and their binding specificities are largely undisturbed by reagents such as detergents and other mild denaturants. Moreover, they are relatively cheap to isolate and produce. They may also readily be modified to generate species with improved properties. Extensive studies show that nucleic acids are largely non-toxic and non-immunogenic and aptamers have already found clinical application. Furthermore, it is known how to modulate the activities of aptamers in biological samples by the production of inactive dsRNA molecules in the presence of complementary RNA single strands (Rusconi et al., 2002).

It is known from the prior art how to isolate aptamers from degenerate sequence pools by repeated cycles of binding, sieving and amplification. Such methods are described in U.S. Pat. No. 5,475,096, U.S. Pat. No. 5,270,163 and EP0533 38 and typically are referred to as SELEX (Systematic Evolution of Ligands by EX-ponential Enrichment). The basic SELEX system has been modified for example by using Photo-SELEX where aptamers contain photo-reactive groups capable of binding and/or photo cross-linking to and/or photo-activating or inactivating a target molecule. Other modifications include Chimeric-SELEX, Blended-SELEX, Counter-SELEX, Solution-SELEX, Chemi-SELEX, Tissue-SELEX and Transcription-free SELEX which describes a method for ligating random fragments of RNA bound to a DNA template to form the oligonucleotide library. However, these methods even though producing enriched ligand-binding nucleic acid molecules, still produce unstable products. In order to overcome the problem of stability it is known to create enantiomeric “spiegeimers” (see for example WO 01/92566). The process involves initially creating a chemical mirror image of the target, then selecting aptamers to this mirror image and finally creating a chemical mirror image of the SELEX selected aptamer. By selecting natural RNAs, based on D-ribose sugar units, against the non-natural enantiomer of the eventual target molecule, for example a peptide made of D-amino acids, a spiegelmer directed against the natural L-amino acid target can be created. Once tight binding aptamers to the non-natural enantiomer target are isolated and sequenced, the Laws of Molecular Symmetry mean that RNAs synthesised chemically based on L-ribose sugars will bind the natural target, that is to say the mirror image of the selection target. This process is conveniently referred to as reflection-selection or mirror selection and the L-ribose species produced are significantly more stable in biological environments because they are less susceptible to normal enzymatic cleavage, i.e. they are nuclease resistant.

Cell line/Cell culture: A “cell line” or “cell culture” denotes higher eukaryotic cells gown or maintained in vitro. It is understood that the progeny of a cell may not be completely identical (either morphologically, genotypically, or phenotypically) to the parent cell. “Heterologous” means derived from a genotypically distinct entity from the rest of the entity to which it is being compared. For example, a polynucleotide may be placed by genetic engineering techniques into a plasmid or vector derived from a different source, and is a heterologous polynucleotide. A promoter removed from its native coding sequence and operatively linked to a coding sequence with which it is not naturally found linked is a heterologous promoter. An “isolated” polynucleotide or polypeptide is one that is substantially free of the materials with which it is associated in nature. By substantially free is meant at least 50%, preferably at least 70%, more preferably at least 80%, and even more preferably at least 90% free of the materials with which it is associated in nature.

Epitope: The site on an antigen recognized by an antibody as determined by the specificity of the amino acid sequence. Two antibodies are said to bind to the same epitope if each competitively inhibits (blocks) binding of the other to the antigen as measured in a competitive binding assay (see, e.g., Junghans et al., Cancer Res. 50: 1495-1502, 1990). Alternatively, two antibodies have the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

Fibronectin: is a large multidomain glycoprotein found in connective tissue, on cell surfaces, and in plasma and other body fluids. It interacts with a variety of macromolecules including components of the cytoskeleton and the extracellular matrix, circulating components of the blood clotting, fibrinolytic, acute phase and complement systems. It is involved in many cellular processes, including tissue repair, embryogenesis, blood clotting and cell migration/adhesion. It is expressed on the surface of activated endothelial cells.

Framework region (FR): Relatively conserved sequences flanking the three highly divergent complementarity-determining regions (CDRs) within the variable regions of the heavy and light chains of an antibody. Hence, the variable region of an antibody heavy or light chain consists of a FR and three CDRs. Some FR residues may contact bound antigen; however, FRs are primarily responsible for folding the variable region into the antigen-binding site, particularly the FR residues directly adjacent to the CDRs. Without being bound by theory, the framework region of an antibody serves to position and align the CDRs. The sequences of the framework regions of different light or heavy chains are relatively conserved within a species. A “human” framework region is a framework region that is substantially identical (about 85% or more, usually 90-95% or more) to the framework region of a naturally occurring human immunoglobulin.

Immunoglobulin: A protein including one or more polypeptides substantially encoded by immunoglobulin genes. The recognized immunoglobulin genes include the kappa, lambda, alpha (IgA), gamma (IgG1, IgG2, IgG3, IgG4), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH2-terminus (about 110 amino adds in length) and a kappa or lambda constant region gene at the COOH—terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17: 105, 1987; Huston et al., Proc. Natl. Acad. Sci. U.S.A., 85: 5879-5883, 1988; Bird et al., Science 242: 423-426, 1988; Hood et al., Immunology, Benjamin, N.Y., 2nd ed., 1984; Hunkapiller and Hood, Nature 323: 15-16, 1986).

An immunoglobulin light or heavy chain variable region includes a framework region interrupted by three hypervariable regions, also called complementarity determining regions (CDR's) (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervarlable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk. 1. Mol. Biol. 196:901-917 (1987)).

The CDRs are primarily responsible for binding to an epitope of an antigen. The term “hypervariable region” when used herein refers to the amino acid residues of an antibody which are responsible for antigen-binding. “Framework” or “FR” residues are those variable domain residues other than the hypervariable region residues as herein defined.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody although other mammalian species can be used, or the variable region can be produced by molecular techniques. Methods of making chimeric antibodies are well known in the art, e.g. see U.S. Pat. No. 5,807,715, which is herein incorporated by reference.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i. e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs.

The acceptor framework of a humanized immunoglobulin or antibody may have a limited number of substitutions by amino acids taken from the donor framework.

Humanized or other monoclonal antibodies can have additional conservative amino acid substitutions which have substantially no effect on antigen binding or other immunoglobulin functions. Exemplary conservative substitutions are those such as Gly, Ala; Val, Ile, Leu; Asp, Glu; Asn, Gln; Ser, Thr; Lys, Arg; and Phe, Tyr (see U.S. Pat. No. 5,585,089, which is incorporated herein by reference). Humanized immunoglobulins can be constructed by means of genetic engineering, e.g. see U.S. Pat. No. 5,225,539 and U.S. Pat. No. 5,585,089, which are herein incorporated by reference.

A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g. Dower et al., PCT Publication No. WO91/17271; McCafferty et al., PCT Publication No. WO92/001047; and Winter, PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (e.g., see Lonberg et al. PCT Publication No. WO93/12227; and Kucherlapati, PCT Publication No. WO91/10741, which are herein incorporated by reference.)

Monoclonal antibody: An antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells.

Immunoreactivity: A measure of the ability of an agent, sometimes an antibody, to recognize and bind to a specific antigen. “Specifically binds” refers to the ability of individual agents or antibodies to specifically immunoreact with an antigen. This binding is a non-random binding reaction between an agent, for example but not limited to a antibody molecule, and the antigen. In one embodiment, the antigen is glycoprotein VI (GPVI), fibronectin and/or vitronectin. Binding specificity is typically determined from the reference point of the ability of the agent to differentially bind the antigen of interest and an unrelated antigen, and therefore distinguish between two different antigens, particularly where the two antigens have unique epitopes.

Typically, specificity may be determined by means of a binding assay such as ELISA employing a panel of antigens. An agent according to the present invention may recognise GPVI on cells of the platelet/megakaryocyte lineage, and not other human blood cells, in particular granulocytes, lymphocytes and erythrocytes.

Inhibit: A species which retards, blocks or prevents an interaction, for example binding between GPVI and a ligand or substrate, is considered to inhibit the interaction. Typically, inhibition does not result in 100% blockage but rather reduces the amount and/or speed of interaction. Thus inhibition of interaction of platelets with the surface of a blood vessel may not completely prevent binding and/or activation of platelets but will desirably have a clinically useful effect in slowing and/or reducing the severity of atherosclerosis.

Low molecular weight compounds: the term will be understood by those skilled in the art. The term may also be understood to include any chemical compound which possesses a molecular weight of below 2,000, e.g. below 1,000.

Low molecular weight thrombin inhibitor: the term will be understood by those skilled in the art. The term may also be understood to include any composition of matter (e.g. chemical compound) which inhibits thrombin to an experimentally determinable degree in in vivo and/or in in vitro tests, and which possesses a molecular weight of below 2,000, e.g. below 1,000.

Particular low molecular weight thrombin inhibitors include low molecular weight peptide-based, amino acid-based, and/or peptide analogue-based, thrombin inhibitors.

The term “low molecular weight peptide-based, amino acid-based, and/or peptide analogue-based, thrombin inhibitors” will be well understood by one skilled in the art to include low molecular weight thrombin inhibitors with one to four peptide linkages, and includes those described in the review paper by Claesson in Blood Coagul. Fibrin. (1994) 5, 411, as well as those disclosed in U.S. Pat. No. 4,346,078; International Patent Applications WO 93/11152, WO 93/18060, WO 93/05069, WO 94/20467, WO 94/29336, WO 95/35309, WO 95/23609, WO 96/03374, WO 96/06832, WO 96/06849, WO 96/25426, WO 96/32110, WO 97/01338, WO 97/02284, WO 97/15190, WO 97/30708, WO 97/40024, WO 97/46577, WO 98/06740, WO 97/49404, WO 97/11693, WO 97/24135, WO 97/47299, WO 98/01422, WO 98/57932, WO 99/29664, WO 98/06741, WO 99/37668, WO 99/37611, WO 98/37075, WO 99/00371, WO 99/28297, WO 99/29670, WO 99/40072, WO 99/54313, WO 96/31504, WO 00/01704 and WO 00/08014; and European Patent Applications 648 780, 468 231, 559 046, 641 779, 185 390, 526 877, 542 525, 195 212, 362 002, 364 344, 530 167, 293 881, 686 642, 669 317, 601 459 and 623 596, the disclosures in all of which documents are hereby incorporated by reference.

Low molecular weight peptide-based thrombin inhibitors include HOOC—CH₂—(R)Cha-Pic-Nag-H (wherein Cha represents cyclohexylalanine, Pic represents (S)-pipecolinic acid and Nag represents noragmatine; known as inogatran; see international Patent Application WO 93/11152) and, especially, HOOC—CH₂—(R)Cgl-Aze-Pab-H (known as melagatran; see above and International Patent Application WO 94/29336).

The term “prodrug” of a low molecular weight thrombin inhibitor includes any compound that releases a low molecular weight thrombin inhibitor, for example one that, following oral or parenteral administration, is metabolised in vivo to form a low molecular weight thrombin inhibitor (as defined herein), in an experimentally-detectable amount, and within a predetermined time (e.g. within a dosing interval of between 6 and 24 hours (i.e. once to four times daily)), following oral or parenteral administration. Prodrugs of the thrombin inhibitor melagatran that may be mentioned include those disclosed in international patent application WO 97/23499. Preferred prodrugs of melagatran are those of the formula R¹O₂C—CH₂—(R)Cgl-Aze-Pab-OH (see the list of abbreviations above or in WO 97/23499), wherein R¹ represents C₁₋₁₀ alkyl or benzyl, such as linear or branched C₁₋₆alkyl (e.g. C₁₋₄ alkyl, especially methyl, propyl and, particularly, ethyl) and the OH group replaces one of the amidino hydrogens in Pab.

Low molecular weight thrombin inhibitors also include Cbz-(R)-Phe-(S)-Pro-(R)-boroMpg (also known as TRI 50c) and its salts and prodrugs, where Cbz represent benzyloxycarbonyl, Phe and Pro have their normal meanings, and boroMpg represents methoxypropyl glycine in which the carboxy group has been replaced by a boronyl group —B(OH)₂. Prodrugs of boronic acids include species in which the boronyl group is reversibly derivatised. See for example WO 2004/022072, U.S. Ser. No. 10/659,178, EP-A-1396270, WO 2004/022071, US 2004/0147453, EP-A-1396269, WO 2004/022070, US 2004/0138175, EP-A-1400245, WO 2005/084685, WO 2005/084686, U.S. Ser. No. 11/078,097, all of which are hereby incorporated by reference.

Nucleic Acid: A “nucleic add” is a polymeric form of nucleotides of any length, which contain deoxyribonucleotides, ribonucleotides, and analogs in any combination. Nucleic acids may have any three-dimensional structure, and may perform any function, known or unknown. The term “nucleic acid” includes double-, single-stranded, and triple-helical molecules. Unless otherwise specified or required, any embodiment of the invention described herein that is a nucleic acid encompasses both the double-stranded form and each of two complementary single-stranded forms known or predicted to make up the double stranded form.

Pathological: a pathological or dysfunctional interaction of GPVI with fibronectin and/or vitronectin and/or collagen represents an interaction which is abnormal compared to the interaction in healthy subjects during the normal function of the body. A pathological or dysfunctional interaction may result in disease in a subject.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “fragment” refers to a portion of a polypeptide that is at least 8, 10, 15, 20 or 25 amino acids in length. The term “functional fragments of a polypeptide” refers to all fragments of a polypeptide that retain an activity of the polypeptide (e.g., the binding of an antigen). Biologically functional fragments, for example, can vary in size from a polypeptide fragment as small as an epitope capable of binding an antibody molecule to a large polypeptide capable of participating in the characteristic induction or programming of phenotypic changes within a cell. The term “soluble” refers to a form of a polypeptide that is not inserted into a cell membrane.

Pharmaceutical agent or drug: A chemical or biological compound or composition capable of inducing a desired therapeutic or prophylactic effect when properly administered to a subject.

Pharmaceutically acceptable carriers: The pharmaceutically acceptable carriers useful in the methods disclosed herein are conventional. Remington's Pharmaceutical Sciences, by E. W. Martin, Mack Publishing Co, Easton, Pa., 15th Edition (1975), describes compositions and formulations suitable for pharmaceutical delivery of the IL-2 receptor antagonists herein disclosed.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

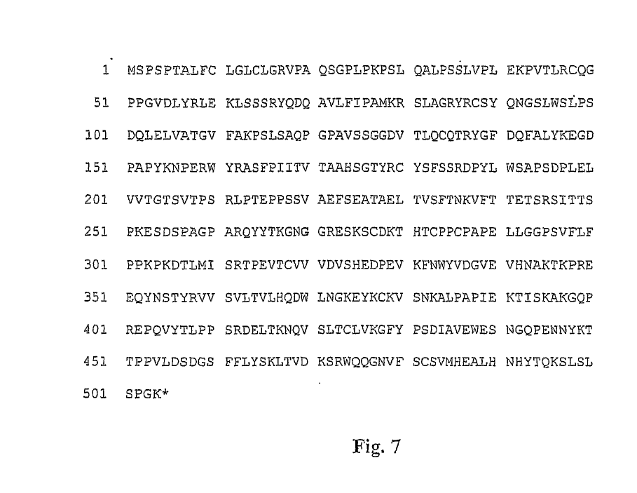

PR-15: a dimer of a protein having the sequence of SEQ ID NO 1 (see FIG. 7).

Purified: The term purified does not require absolute purity or isolation; rather, it is intended as a relative term. Thus, for example, a purified or isolated protein preparation is one in which protein is more enriched than the protein is in its generative environment, for instance within a cell or in a biochemical reaction chamber. Preferably, a preparation of protein is purified such that the protein represents at least 50% of the total protein content of the preparation. For pharmaceuticals, “substantial” purity of 90%, 95%, 98% or even 99% or higher of the active agent can be utilized.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of the IL-2R antibodies or antigen binding fragments, and the corresponding cDNA sequence, will possess a relatively high degree of sequence identity when aligned using standard methods. This homology will be more significant when the orthologous proteins or cDNAs are derived from species that are more closely related, compared to species more distantly related (e.g., human and murine sequences).

Methods of alignment of sequences for comparison are well known in the art.

Various programs and alignment algorithms are described in Smith and Waterman, Adv. Appl. Math. 2: 482, 1981; Needleman and Wunsch, J. Mol. Biol. 48: 443, 1970; Pearson and Upman, Proc. Natl. Acad. Sd. U.S.A. 85: 2444, 1988; Higgins and Sharp, Gene 73: 237-244 9, 1988); Higgins and Sharp, CABIOS 5: 151-153, 1989; Corpet et al., Nuc. Acids Res. 16: 10881-90, 1988; Huang et al., Computer Appls. In the Biosclences 8: 155-65, 1992; and Pearson et al., Meth. Mol. Bio. 24: 307-31, 1994. Altschul et al., J. Mol. Biol. 215: 403-410, 1990, presents a detailed consideration of sequence alignment methods and homology calculations.

A sequence described herein with reference to a wild type sequence, for example GPVI, GPVI extracellular domain or Fc, may unless otherwise specified have a nature-identical sequence or it may be a non-natural mutant; it may comprise a fragment of a natural or non-natural protein. The sequence may comprise or consist of a sequence having at least 90% homology with a wild type sequence, e.g. at least 91%, 92%, 93%, 94%, 95%, 96%, 97%, 98% or 99%.

As used herein, “percent homology” of two amino acid sequences or of two nucleic acid sequences is determined using the algorithm of Karlin and Altschul (Proc. Natl. Acad. Sd. USA 87:2264-2268 (1990)). Such an algorithm is incorporated into the NBLAST and XBLAST programs of Altschul et al. (J. Mol. Biol. 215:403-410 (1990)). BLAST nucleotide searches are performed with the NBLAST program, score 100, wordiength=12, to obtain nucleotide sequences homologous to a nucleic acid molecule of the invention. BLAST protein searches are performed with the XBLAST program, score=50, wordlength=3, to obtain amino acid sequences homologous to a reference polypeptide. To obtain gapped alignments for comparison purposes, Gapped Blast is utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://www.ncbl.nlm.nih.aov.

Treatment As used herein, “treatment” refers to clinical intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, lowering the rate of disease progression, amelioration or palliation of the disease state, and remission or improved prognosis. Unless otherwise specified or required by the context, treatment may be therapeutic, for example, restoring vessel patency, or prophylactic, for example preventing thrombosis or initiation or development of atherosclerosis.

Variable region (also variable domain or V domain): The regions of both the light chain and the heavy chain of an Ig that contain antigen-binding sites. The regions are composed of polypeptide chains containing four relatively invariant “framework regions” (FRs) and three highly variant “hypervariable regions” (HVs). Because the HVs constitute the binding site for antigen(s) and determine specificity by forming a surface complementarity to the antigen, they are more commonly termed the “complementarity-determining regions,” or CDRs, and are denoted CDR1, CDR2, and CDR3. Because both of the CDRs from the heavy and light chain domains contribute to the antigen-binding site, it is the three-dimensional configuration of the heavy and the light chains that determines the final antigen specificity.

Within the heavy and light chain, the framework regions surround the CDRs. Proceeding from the N-terminus of a heavy or light chain, the order of regions is: FR1-CDR1-FR2-CDR2-FR3-CDR3-FR4. As used herein, the term “variable region” is intended to encompass a complete set of four framework regions and three complementarity-determining regions. Thus, a sequence encoding a “variable region” would provide the sequence of a complete set of four framework regions and three complementarity-determining regions.

Vectors As used herein, a “vector” may be any of a number of nucleic acids into which a desired sequence may be inserted. Vectors include, but are not limited to, plasmids, phagemids and virus genomes. A cloning vector is one which is able to replicate in a host cell, and which typically is further characterized by one or more endonuclease restriction sites at which the vector may be cut in a determinable fashion and into which a desired DNA sequence may be ligated such that the recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

Vectors may further contain one or more selectable marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., p-galactosidase, luciferase), and genes which visibly affect the phenotype of transformed or transfected cells, hosts, colonies or plaques (e.g., various fluorescent proteins such as green fluorescent protein, GFP). Preferred vectors are those capable of autonomous replication, also referred to as episomal vectors. Alternatively vectors may be adapted to insert into a chromosome, so called integrating vectors. The vector of the invention is typically provided with transcription control sequences (promoter sequences) which mediate cell/tissue specific expression. These promoter sequences may be cell/tissue specific, inducible or constitutive.

Promoter is a term recognised in the art and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites, environmental effectors.

Promoter elements also include so called TATA box, RNA polymerase initiation selection (RIS) sequences and CAAT box sequence elements which function to select a site of transcription initiation. These sequences also bind polypeptides which function, inter alia, to facilitate transcription initiation selection by RNA polymerase.

Adaptations also include the-provision of autonomous replication sequences which both facilitate the maintenance of said vector in either the eukaryotic cell or prokaryotic host, so called “shuttle vectors”. Vectors which are maintained autonomously are referred to as episomal vectors. Episomal vectors are desirable since these molecules can incorporate large DNA fragments (30-SOkb DNA).

Vitronectin: Vitronectin is a mixture of two monomeric glycoproteins (65 and 75 kD) present in blood and the extracellular matrix of many tissues. Vitronectin is expressed on the surface of activated endothelial cells. Vitronectin is one of the major adhesive proteins in plasma and serum. Vitronectin has been considered to be involved in promoting cell attachment, spreading, proliferation, and differentiation of many normal and neoplastic cells, and has been used to study cell migration.

### Overview and Highlights

The present invention is further described below without limitation.

The disclosure includes diverse products which are capable of inhibiting binding of one or more components of the blood to one or more potential binding sites for them in the cardiovascular system.

There are disclosed herein inter alia products comprising agents which inhibit one or both of:
GPVI interaction with fibronectin
GPVI interaction with vitronectin.

The agents may inhibit GPVI binding to the ligand molecule(s) (i.e. fibronectin, vitronectin, or both). One class of agents binds to GPVI. Another class of agents binds to one or both of the ligand molecules.

Some agents bind to GPVI at a binding site of GPVI for the ligand(s) concerned.

Alternative agents bind to the ligand(s) concerned, optionally at a GPVI binding site of the ligand(s). Thus, there are included agents which bind to vitronectin, agents which bind to fibronectin and agents which bind to both of fibronectin and vitronectin.

The disclosure thus includes agents which inhibit GPVI interaction with fibronectin. The agents may inhibit GPVI binding to fibronectin. In some embodiments, the agents bind to fibronectin, optionally, for example, at a GPVI binding site of fibronectin. It is proposed that inhibiting the interaction between GPVI and fibronectin inhibits platelet adhesion; it is proposed that such activity inhibits platelet aggregation.

The disclosure includes agents which inhibit GPVI interaction with vitronectin. The agents may inhibit GPVI binding to vitronectin. In some embodiments, the agents bind to vitronectin, optionally, for example, at a GPVI binding site of vitronectin. It is proposed that inhibiting the interaction between GPVI and vitronectin inhibits platelet adhesion; it is proposed that such activity inhibits platelet aggregation.

The present specification discloses the role of GPVI interaction with exposed sub-endothelial matrix and/or adhesion proteins other than collagen.

Embodiments of the present disclosure reside in a protein which binds to collagen at a site at which membrane bound GPVI binds to collagen. Embodiments of the disclosure reside in a protein which binds to fibronectin at a site at which membrane-bound GPVI binds to fibronectin. Embodiments of the disclosure reside in a protein which binds to vitronectin at a site at which membrane bound GPVI binds to vitronectin.

As will become apparent, there may particularly be mentioned, as examples of the products described in the preceding paragraphs, species comprising an amino acid sequence of, or derived from, an extracellular domain of GPVI. Other examples include antibody products (e.g. antibodies, antibody fragments, engineered antibodies and antibody fragments), aptamers and molecules derived from snake venom.

There are further disclosed herein products, for example molecules, which bind to or interact with plural vascular domains or binding sites. Typically at least one of the domains, and more normally all the domains (or binding sites), are potential sites of interaction with platelets; in embodiments, therefore, the products can hinder or block tethering or binding of platelets to blood vessel walls having such domains or binding sites Each domain may comprise a different ligand, for example adhesion protein, from each other domain. The products are exemplified below by products of which at least one of the domains binds to or interacts with GPVI. In one class of products, all the binding sites bind to or interact with GPVI. The products mentioned in this paragraph can provide enhanced binding properties as compared to single domain-binding products, for example enabling adhesion to a blood vessel wall or atherosclerotic plaque for a period of at least 12 hours, particularly at least 24 hours, e.g. 48 hours or more as explained next.

Example 10 and 11 describe that PR-15 binds to injured blood vessels for at least 48 hours after intravenous administration. PR-15 is a representative of agents interacting with plural vascular domains (in this case selected from collagen, vitronectin and fibronectin) and the data indicate that such agents may advantageously be administered at a frequency of twice daily or less, e.g. at periods of once a day or longer, e.g. once every two, three or four days or once a week. Methods involving administration at such periods or more are included in the disclosure.

Advantageously in the case of plural domain binding products as mentioned in the preceding paragraph, the domains or binding sites may simultaneously be exposed on a dysfunctional or diseased area of blood vessel wall, for example an atherosclerotic or inflamed area. The products may, as already indicated, function when bound to a vascular surface to retard or prevent tethering and/or adhesion of platelets; particular embodiments relate to products which are capable of binding to a dysfunctional or inflamed area of blood vessel wall which does not include an atherosclerotic plaque or lesion; such products can have value in inhibiting, for example preventing or retarding, Initiation of atherosclerosis.

The disclosure includes agents which are capable of binding to a plurality of GPVI-binding sites of a dysfunctional, inflamed or atherosclerotic blood vessel area, for example a human blood vessel area. The binding sites may all be of different types; alternatively stated, they may all comprise different ligands, e.g. adhesion proteins. Exemplary are agents, or products, which are capable of binding to two or three ligands selected from collagen, vitronectin and fibronectin. Such products are exemplified below by products (agents) which can bind to all of collagen, fibronectin and vitronectin.

Accordingly, an aspect of the invention is products which are capable of binding to all of collagen, fibronectin and vitronectin. However, certain agents which bind to as few as a single one of these proteins are also included in the disclosure, as described next.

Turning now to the disclosure in more detail, the present specification identifies that GPVI, bound to the surface of platelets, binds to vitronectin and fibronectin. There are many different types of receptors on platelets, of which GPVI is one. Some bind small molecules such as epinephrine or thromboxane A2 (TxA2), which help activate the platelet. Other receptors on the surface of platelets help the platelet tether and/or adhere to sites of vascular injury but also help tethering and/or adherence to atherosclerotic blood vessel areas. These include the receptors for collagen (GPIa/IIa, GPIIb/IIIa, and GPIV), fibronectin (GPIc/IIa), fibrinogen (GPIIb/IIIa), vitronectin (GPIIb/IIIa), and von Willebrand factor (GPIb/IX). Finally, other receptors help platelets aggregate to form a clump at the site of vessel injury, which ultimately stops the bleeding from the site. These mostly include fibrinogen and the GPIIb/IIIa receptor, for which there are about 50,000 complexes on the surface of an inactivated platelet. Once activated, these complexes avidly bind fibrinogen/fibrin, which acts as a scaffold that aggregates platelets together.

Fibronectin and vitronectin are proteins which are expressed on the surface of activated endothelial cells. The endothelium may be activated in response to or association with many stimuli, including inflammation. Fibronectin and vitronectin are present on dysfunctional endothelium. Without being bound by theory, the present disclosure proposes inter alia use of an agent capable of binding at least to one or both of fibronectin and vitronectin to inhibit interaction between GPVI on membrane bound platelets and an inflamed or dysfunctional endothelium.

Platelets briefly adhere to an activated but intact endothelium but tend not to adhere strongly but to subsequently ‘bounce away’ from the endothelium. The brief adherence of platelets to the activated endothelium may activate platelets which subsequently release activatory molecules. Whilst not being bound by theory, it is considered that vitronectin and fibronectin play a role in platelet binding to the endothelium, in particular via GPVI. At present, vitronectin and fibronectin are considered not to activate platelets by themselves, or to have a minor role in platelet activation.

The adhesion of platelets to an activated, e.g. dysfunctional, but intact endothelium may be a first step in the formation of an atherosclerotic plaque. Inhibition of this interaction may prevent the subsequent activation of platelets which have adhered, albeit perhaps briefly, to the activated endothelium. Thus, agents of the present disclosure as well as methods and uses thereof may be useful in preventing initiation of atherosclerotic lesions or plaques. The agents may act to inhibit the adhesion of platelets to an activated endothelium by inhibition of interaction between GPVI and fibronectin and/or vitronectin. The present disclosure has therefore new therapeutic applications in patients who do not yet show symptoms of atherosclerotic plaque but who are potentially at risk of developing atherosclerotic plaques in the future. Such patients may have been identified as having a risk factor for cardiovascular disease and/or who are at risk of suffering from cardiovascular disease, e.g. coronary heart disease.

Patients who are at risk of developing atherosclerotic lesions can be identified using risk assessment calculations. Such risk assessment calculations may include the PROCAM coronary heart disease risk function and the Framingham coronary heart disease risk function. The PROCAM risk function estimates the probability of developing coronary death or first myocardial infarction within ten years and employs age, systolic blood pressure, LDL and HDL cholesterol, triglycerides, cigarette use, diabetes and family history of myocardial infarction as risk factors.

The FRAMINGHAM risk function estimates the probability of developing coronary death, myocardial infarction (recognised and unrecognised), angina pectoris or coronary insufficiency (total CHD end points) within ten years taking age, blood pressure, LDL and HDL cholesterol, cigarette use and diabetes as risk factors. (Anderson K M et al, Circulation 1991; 83:356-362). Thus, agents of the present disclosure may be used in the treatment, particularly chronic or long-term treatment, of patients who have been identified as having a risk factor of 45 or more. Thus, agents of the present invention may be administered to patients who have been identified as having a PROCAM score of 45, 46, 47, 48, 49, 50, 51, 52, 53, 54, 55, 56, 57, 58, 59 or 60. Such treatment is prophylactic, to prevent, delay or reduce the risk of future cardiovascular disease.

Methods, uses and agents of the present disclosure can be utilised for or in the treatment of patients who are at risk of development of atherosclerotic plaques. Such patients may have been identified as having some or all of the risk factors associated with atherosclerosis. Thus, one class of patients which could be treated using agents, methods and uses of the present invention are patients who have been identified using the Framingham risk factor as having a defined level of risk of developing atherosclerotic lesions and complications thereof. Thus, the defined level of risk may be determined according to the points obtained by the patient using the Framingham method. Thus, in one embodiment of the present invention, there is provided a method of treating a male patient who has been identified as having a points score according to the Framingham study of 11 or more. Thus, the male patient may have a Framingham points score of 11, 12, 13, 14, 15, 16, 17 or greater. In the above embodiment the patient is a male subject. Alternatively, when the patient is a female subject, the patient may have been identified as having a risk factor, using the Framingham study, wherein the patient has been awarded 20 or more points according to the Framingham point scores. Thus, the present disclosure the female patient may have 20, 21, 22, 23, 24, 25 or more according to the Framingham points score.

Another way of assessing cardiovascular risk is the SCORE (Systematic COronary Risk Evaluation) risk estimation system, established under the SCORE project funded under the European Union BIOMED programme. A patient may be considered at risk justifying prophylactic medication if he/she has a 10-year risk of fatal cardiovascular disease according to the SCORE project of at least 3%, for example at least 5 as in the case of patients having a risk of at least 10%.

It will be appreciated from the aforegoing that the disclosure includes primary prophylaxis of cardiovascular disease using an agent which inhibits interaction of GPVI with one or both of fibronectin and vitronectin.

In another aspect there is provided a method of treating a patient who has previously suffered a cardiovascular event associated with, or suspected of being associated with, atherosclerosis (e.g. plaque rupture, disruption or fissure), wherein the method comprises administering an effective amount of an agent of the present disclosure.

As well as binding to the fibronectin and vitronectin, GPVI also binds to collagen. In particular, GPVI binds to collagen Types I and III. Immunohistochemical studies are reported to have shown enrichment of collagen types I, III, V, and VI, vitronectin, fibronectin, fibrinogen/fibrin, and thrombospondin in the atherosclerotic plaque. The GPVI ligands collagen (specifically collagen I and collagen III), vitronectin and fibronectin are therefore exposed when an atherosclerotic lesion is disrupted and/or ruptures. Collagen and adhesion proteins, including vitronectin and fibronectin, are also thought to be exposed for some time after rupture or fissure of the plaque, when the lesion is healing. Thus, platelets bind to GPVI-binding sites (to be mentioned are collagen, vitronectin and fibronectin) when an atherosclerotic plaque ruptures or fissures, become activated and cause aggregation of platelets at the site of atherosclerotic rupture or disruption. Thus, inhibition of GPVI interaction with one or more of its ligands, e.g. at least collagen, can inhibit thrombus formation at the site of atherosclerotic lesion rupture.

Whilst not being bound by theory, it is believed that GPVI ligands remain exposed for a period after a lesion has ruptured or fissured, and during the repair process of the lesion. As a result, there is a danger of further cardiovascular or atherothrombotic events following an initial cardiovascular event that occurs due to rupture or disruption of an atherosclerotic plaque. Thus, agents of the present disclosure or direct and indirect inhibitors of GPVI may be useful in secondary prophylaxis after a cardiovascular event, as well as in primary prophylaxis against atherosclerosis and/or thrombosis. Advantageously, the agent used in this context is capable of binding to plural GPVI-binding sites (e.g. to all of collagen, vitronectin and fibronectin) to provide relatively strong binding to a ruptured or fissured surface.

Thus, in one aspect, there is provided a method of treating a patient who has suffered a cardiovascular event, particularly one which is or is suspected of being a result of rupture or disruption of an atherosclerotic lesion, comprising administering an effective amount of agent according to the present disclosure, or a GPVI inhibitor, to said patient over an extended period of time. Such an extended period of time may be of at least one month, e.g. from three to six months or more following the initial cardiovascular event. In this instance the treatment of the patient may be considered a chronic treatment. The skilled reader will appreciate that the administration may take place periodically throughout the term of the treatment, e.g. at periods of twice a day, once a day or longer. Substantially continuous administration by, for example, infusion is not excluded. In one embodiment, the mode of administration of the agent of the invention may be intravenous, inter-arterial or subcutaneous injection or infusion, or by oral administration.

It will therefore be understood that there is disclosed a method of treating a patient who has suffered or is suspected to have suffered an atherothrombotic event, the method comprising administering the agent to the patient over a time scale of at least three months, following the initial cardiovascular event. Thus, the method may comprise administering an effective amount of an agent of the disclosure for three, four, five, six or more months following a cardiovascular event, for example thrombosis or myocardial infarction.

Circulating monocytes are among the earliest cells recruited into experimentally induced vascular lesions in animals and spontaneous atherosclerosis in human arteries. As a result, monocytes have been hypothesized to serve as markers, initiators, and promoters of arterial occlusive diseases. It has been reported that monocytes adhere to the luminal surface of stent-injured arteries and then penetrate the healing arterial wall, with resultant neointimal hyperplasia. Mechanisms of monocyte adhesion that have been defined in vitro include vitronectin-dependent binding of urokinase-type plasminogen activator receptors on the cell surface. Monocytes have been shown to adhere to fibronectin. It is contemplated that agents of the invention, particularly agents which are capable of disrupting GPVI interaction with one or both of fibronectin and vitronectin, will inhibit monocyte recruitment. Thus, the agents of the disclosure may be used to inhibit interaction between monocytes and the endothelium thus inhibiting or retarding monocyte recruitment to a site of inflammation on the endothelial. It is contemplated that such activity will help prevent or retard initiation and development of atherosclerosis. It is further contemplated that inhibition of monocyte adhesion and migration into mechanically injured arteries will alter vascular repair, limit the extent of neointimal hyperplasia, and potentially control arteriopathies that follow vascular intervention or injury.

The disclosure includes a method for inhibiting (for example blocking or retarding) monocyte recruitment to an injured, dysfunctional or diseased blood vessel area, comprising contacting the blood vessel area with a product described herein which binds to one or both of vitronectin and fibronectin. The method may be performed in vitro for screening or research purposes, or in vivo for medical purposes (prophylaxis or therapy) by administering an effective amount of such product to a patient. One class of patients has had a cardiovascular intervention, particularly implantation of a cardiovascular device (see later in this specification for further description of such devices).

The disclosure also teaches the unpredicted benefits of co-administering, whether or not by fixed combination, (I) an agent of the disclosure, e.g. an inhibitor of interaction between GPVI and a protein selected from vitronectin, fibronectin and combinations thereof, and, in particular, (ii) at least one other antiplatelet agent (e.g. acetyl salicylic acid or clopidogrel), a direct or indirect matrix metalloprotease inhibitor, and/or an anticoagulant. For example, an agent of the disclosure may be co-administered with acetyl salicylic acid and clopidogrel. Another hitherto unpredicted benefit taught by this disclosure arises from combining or co-administering an anticoagulant, particularly a low molecular weight thrombin inhibitor, with an agent described herein.

An agent described herein may be used in various methods of treating vascular disorders. Advantageously, the agents of the disclosure bind effectively to an atherosclerotic disorder, particularly a ruptured or fissured plaque, when delivered locally, thus avoiding the necessity of systemic administration at clinical levels. Thus, there is disclosed local administration, e.g. by a catheter, of an agent of the disclosure to a site, or suspected site, of an atherosclerotic disorder. In one embodiment, such an agent may be administered to a patient via a catheter to treat, for example, plaque disruption (e.g. rupture, fission), stenosis or restenosis or other cardiovascular disorders. In one embodiment, the agent is a fusion protein comprising an extracellular domain of GPVI fused to an immunoglobulin Fc portion via a linker. In one embodiment, the fusion protein is PR-15, a fusion protein comprising the sequence disclosed in FIG. 7.

The administration of an agent of the present invention via a catheter may provide an alternative method of treating spontaneous or other types of arterial lesions to systemic antiplatelet therapy.

Advantageously, the agents of the disclosure bind to the target molecule with a high degree of affinity. Binding affinity of an agent for example an antibody or fusion protein may be measured using for example BIACORE systems. The strength of the binding of an agent to a peptide sequence of GPVI may be analysed using chemiluminescence and quantified by measuring signal intensity.

### Products

Included in the products of the disclosure are agents having the activities mentioned in this specification.

It is considered that the agents may be, for example, antibodies, antibody fragments, proteins, polypeptides, fusion proteins, aptamers or compounds (particularly low molecular weight compounds) or combinations thereof. Where the agent is an antibody or fragment thereof, the antibody or fragment thereof may be, although is not limited to, a monoclonal antibody, a polyclonal antibody, a chimeric antibody, a human antibody or a humanized antibody and the fragment is a fragment of such an antibody. Other agents which bind the ligands, for example, GPVI, fibronectin and/or vitronectin, as defined herein are encompassed within the present invention.

Also included in the present invention are agents which comprise natural amino acid sequences, e.g. a sequence found in the extracellular domain of GPVI, or variants thereof. Typically, variants are those that vary from a reference (natural) amino acid sequence by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Typically, conservative modifications of a reference amino acid sequence do not affect the biological function of the sequence.

It is considered that, since the GPVI gene has multiple alleles, any reference to a GPVI protein or fragment thereof encompasses all GPVI polymorphisms and expressed GPVI proteins thereof. For example, the proteins encoded by the GPVIa and GPVIb alleles differ by five amino acids. Three of these are in the stem (S219P, K237E, T249A), and two are in the cytoplasmic domain (Q317L and H322N). Both GPVIa and GPVIb are considered “GPVI”.

Also included in the present invention are variants which show enhanced biological activity relative to an original amino acid sequence, for example, a sequence found in the extracellular domain of GPVI. Further definition of an antibody and fragments thereof are included herein and form part of the invention.

In some embodiments, where the agent is an antibody fragment, the antibody fragment is a Fab fragment, a F(ab′)₂ fragment, a scFv, a Fv fragment or a single domain antibody.

The agent may be a humanized antibody or fragment thereof. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical, e.g. at least 96%, 97%, 98% or 99%. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs.

Methods of antibody isolation are well known in the art. See, for example, Harlow and Lane (1988) Antibodies: A Laboratory Manual, Cold Spring Harbor Laboratory, New York. The method of isolation may depend on the immunoglobulin isotype. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (Including gel filtration HPLC), and chromatography on affinity resins such as protein A, protein G, hydroxyapatite, and anti-immunoglobulin. Particularly, the agent of the invention is purified by using Protein G-Sepharose columns.

The agent may also be a single domain antibody such as, for example, a VHH directed to a target mentioned herein, for example, GPVI, collagen, fibronectin and/or vitronectin. The VHH may belong to a class having human-like sequences. The class is characterised in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 according to the Kabat numbering. As such, peptides belonging to this class show a high amino acid sequence homology to human VH framework regions and said peptides might be administered to a human directly without expectation of an unwanted immune response therefrom, and without the burden of further humanisation.

According to an aspect of the present invention, the agent may comprise a polypeptide construct comprising two or more single domain antibodies which have been joined. The single domain antibodies may be identical in sequence and directed against the same target or antigen, for example, GPVI, collagen, fibronectin and/or vitronectin. Depending on the number of VHHs linked, a multivalent VHH may be bivalent (2VHHs), trivalent (3VHHs), tetravalent (4 VHHs) or have a higher valency molecules.

Thus, according to one embodiment, there is provided a single domain antibody directed towards fibronectin. That is to say, there is provided a single domain antibody which is capable of inhibiting fibronectin binding with GPVI. In one embodiment, the single domain antibody binds to fibronectin. In another embodiment, the single domain antibody binds to GPVI and prevents GPVI binding to fibronectin.

Thus, there is provided a single domain antibody directed towards vitronectin. That is to say, there is provided a single domain antibody which is capable of inhibiting vitronectin binding with GPVI. In an embodiment, the single domain antibody binds to vitronectin. In another embodiment, the single domain antibody binds to GPVI and prevents GPVI binding to vitronectin.

Uses of single domain antibodies against fibronectin, vitronectin and GPVI in treating platelet dysfunction disorders are envisaged.

Included in the present disclosure is a protein or polypeptide derived from a snake venom protein and which is capable of inhibiting the interaction between GPVI and fibronectin and/or vitronectin. In a particular embodiment, the snake venom derived protein or polypeptide may bind to GPVI and prevent binding of GPVI to fibronectin. In another particular embodiment, the snake venom derived protein may bind to fibronectin and prevent binding of GPVI to fibronectin.

In a particular embodiment, the snake venom derived protein or polypeptide may bind to GPVI and prevent binding of GPVI to vitronectin. In another particular embodiment, the snake venom derived protein may bind to vitronectin and prevent binding of GPVI to vitronectin.

Snake venoms are mixtures of biologically active proteins and peptides. Many snake venoms affect hemostasis by activating or inhibiting coagulant factors or platelets, or by disrupting the endothelium. Snake venom components can be classified into various families, such as C-type lectins, metalloproteinases, serine proteases, disintegrins and phospholipases. The various members of a particular family may act selectively on different blood coagulation factors, blood cells or tissues (Lu et al; Toxicon. 2005 Jun. 15; 45(8):1089-98)

To be mentioned as potential agents are proteins derived from Crovidisin, a collagen-binding protein isolated from snake venom of Crotalus viridis. For example, such proteins may be recombinant proteins which have homology to Crovidisin and additionally include one or both of a vitronectin-binding domain and a fibronectin-binding domain.

A particular class of agents comprise an amino acid sequence of or comprised in an extracellular domain of GPVI, or a variant thereof that is functional for binding to one, two or three proteins selected from the group consisting of collagen, vitronectin and fibronectin. To be mentioned are such agents which are functional to bind to collagen and to at least one protein selected from fibronectin and vitronectin. Such agents may be soluble in plasma; for example, it is known that glycosylation may be modified, e.g. increased, to improve solubility. Included in the disclosure, therefore, are polypeptides comprising a sequence of or contained in an extracellular domain of GPVI or a function conservative variant of fragment thereof. For example, polypeptides may include a sequence having a homology of at least 90%, e.g. at least 91%, 92%, 93%, 94%, 95%, 96%, 97%, 98% or 99%, with an extracellular domain of GPVI.

Products of the disclosure include formulations which contain a plurality of agents, each targeting a different binding site (e.g. respectively targeting two or three of vitronectin, fibronectin and collagen), e.g. a first agent which interferes with GPVI-vitronectin interaction, a second agent which interferes with GPVI-fibronectin interaction and an optional third agent which interferes with GPVI-collagen interaction.

Methods of the disclosure include co-administration, whether or not in fixed combination, of a plurality of agents (often two or three agents), each targeting a different binding mode, e.g. different vascular binding sites, for platelets, monocytes or other components of blood. For example, there may be administered two or more agents, e.g. two or three agents, each of which interferes with interaction of GPVI with a different one of vitronectin, fibronectin and collagen. Products of the disclosure include formulations, packages or kits comprising a plurality of active agents, each agent being capable of interrupting a different mode of binding, for example different receptor-ligand interaction, of a blood component, particularly a platelet or monocyte. Such a plurality of agents may be used in a method of treatment.

In embodiments, the plurality of agents are all antibody products, for example antibody fragments. Particularly to be mentioned are co-administration and/or combinations (e.g. combination formulations, combination packages, combination kits) of plural (e.g. two or three) single domain antibodies as described above, such as, for example, those referred to by the trade mark Nanobodies. In other embodiments, methods or products involve plural (e.g. two or three) domain antibodies (dAb's) as described above.

Some agents, particularly polypeptides, comprise an amino acid sequence encoded by:
(i) a nucleic acid sequence which encodes a wild type GPVI extracellular domain;
(ii) a nucleic acid sequence which hybridises to said nucleic acid sequence (I) and which encodes a polypeptide that is capable of binding to a plurality of types of GPVI-binding sites of a dysfunctional, inflamed or atherosclerotic blood vessel area; or
(iii) a nucleic acid sequence which differs from said nucleic acid sequence (I) by virtue of the degeneracy of the genetic code.

Also included in the disclosure are agents, particularly polypeptides, which comprise an amino acid sequence encoded by:
(i) a nucleic acid sequence which encodes a wild type GPVI extracellular domain;
(ii) a nucleic add sequence which hybridises to said nucleic acid sequence (I) and which encodes a polypeptide that is which is capable of inhibiting the binding of GPVI to collagen and to one or both of fibronectin and vitronectin; or
(iii) a nucleic acid sequence which differs from said nucleic acid sequence (i) by virtue of the degeneracy of the genetic code.

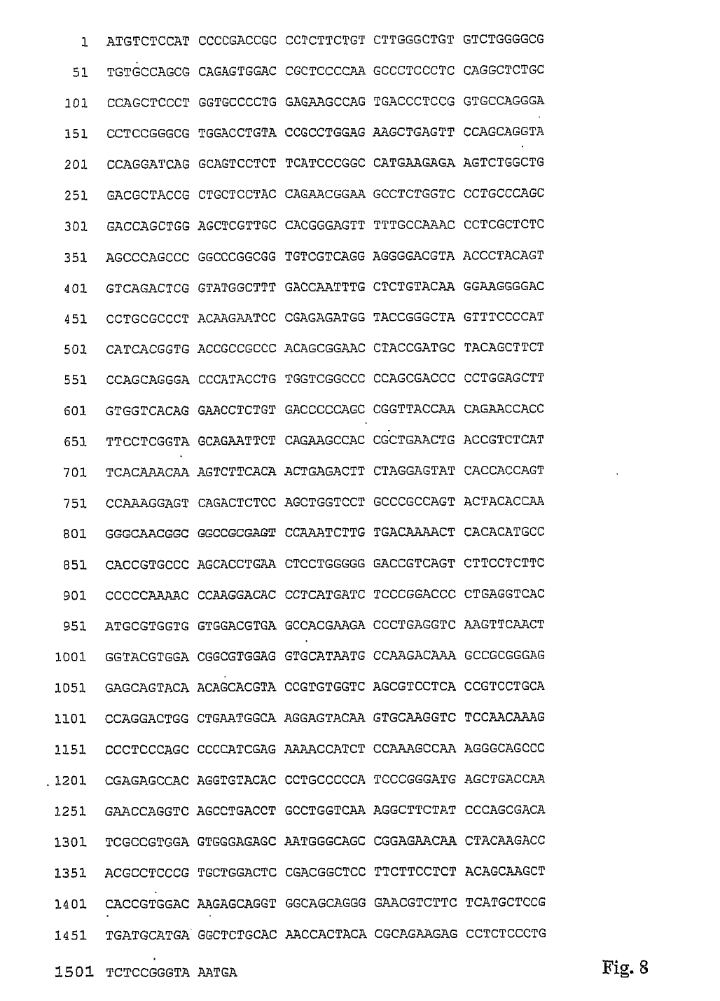

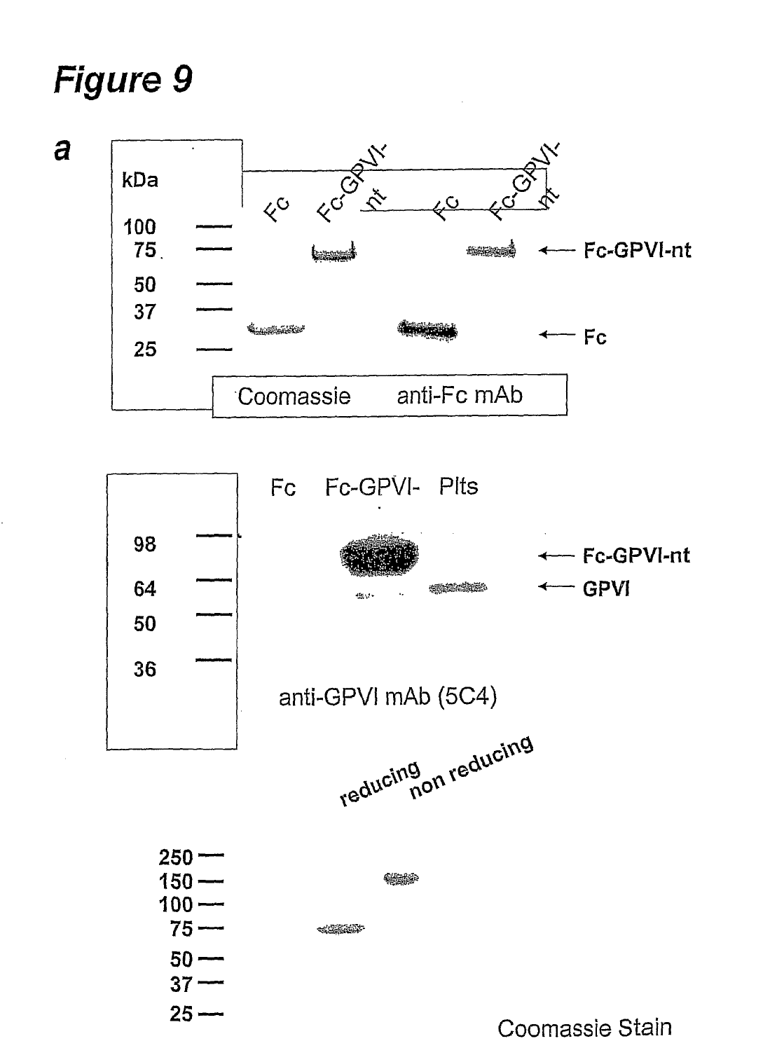

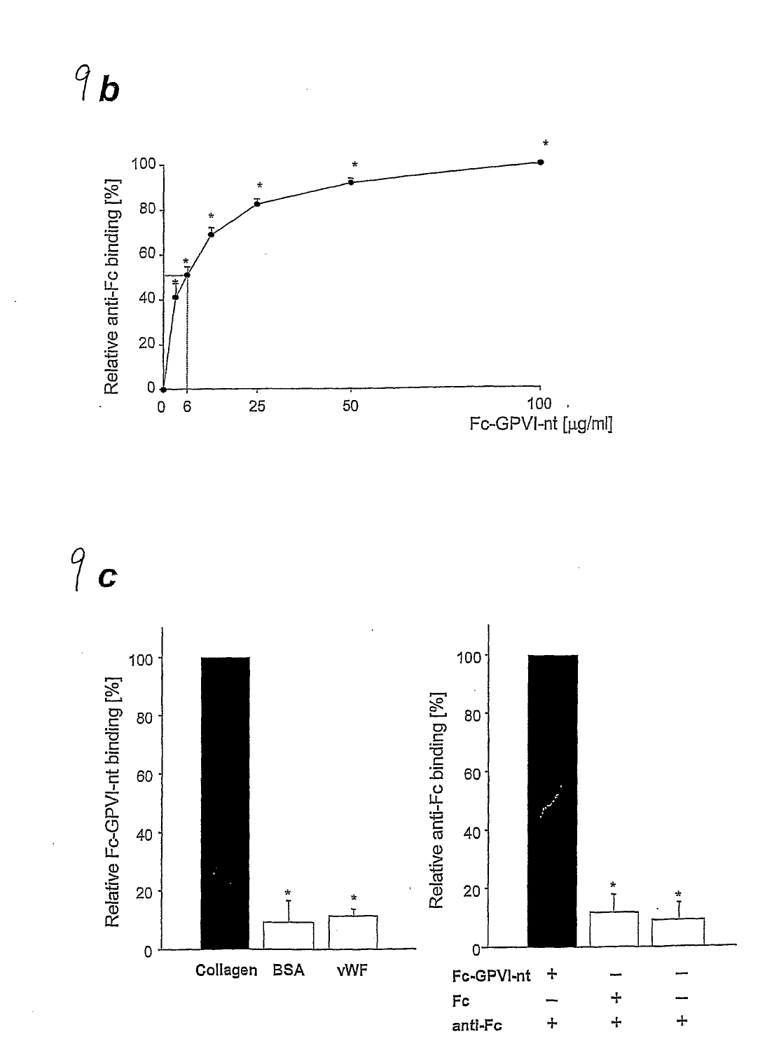

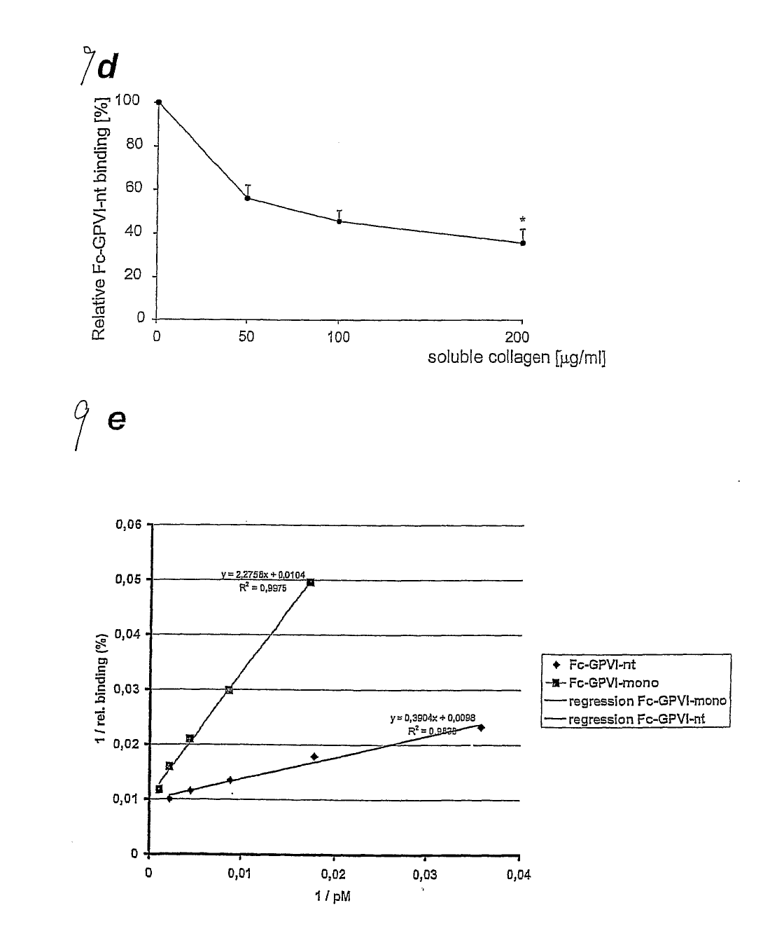

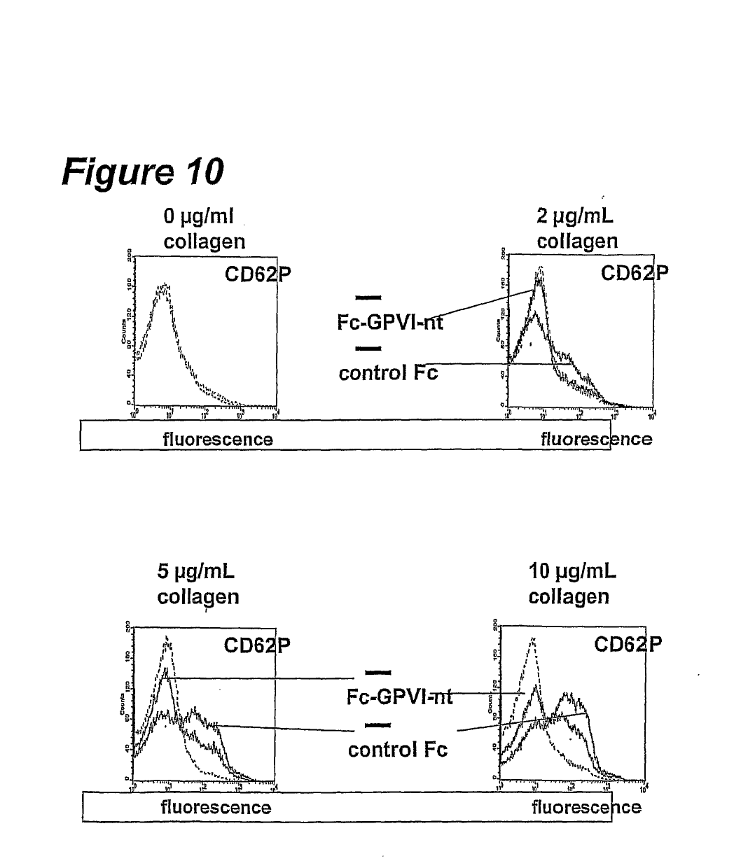

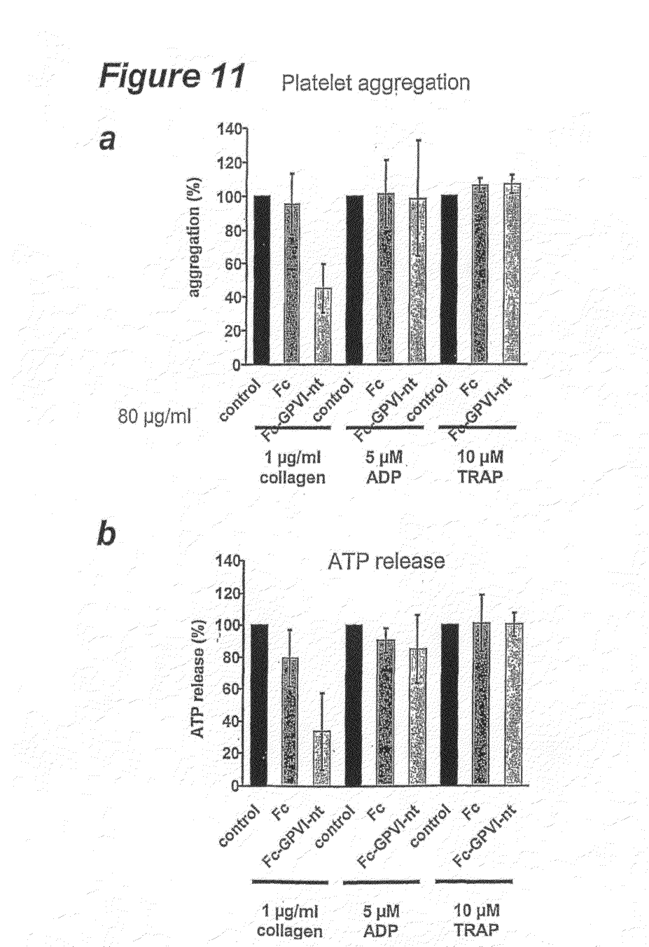

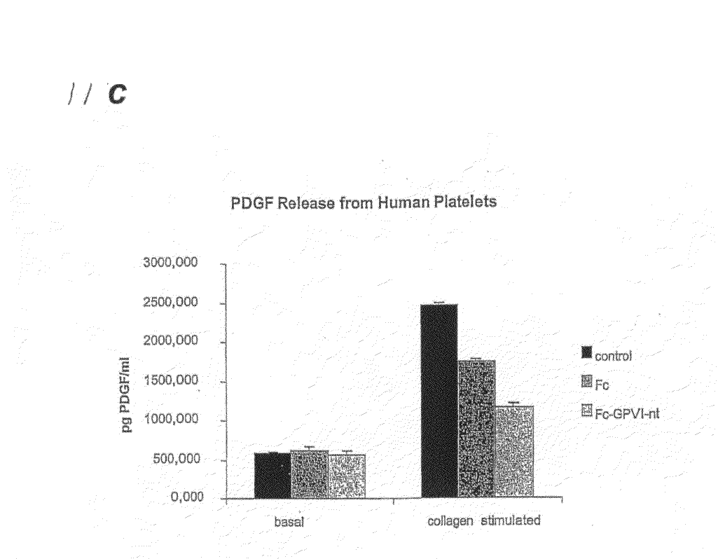

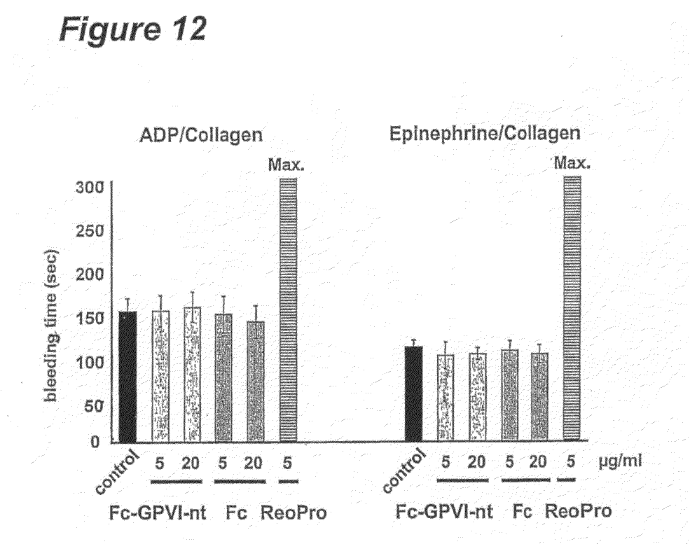

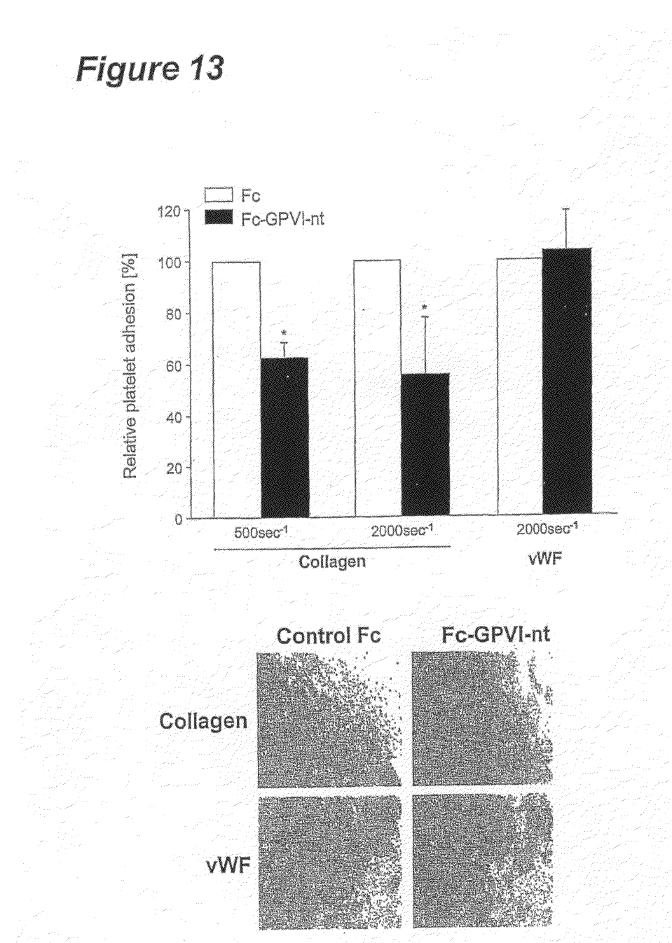

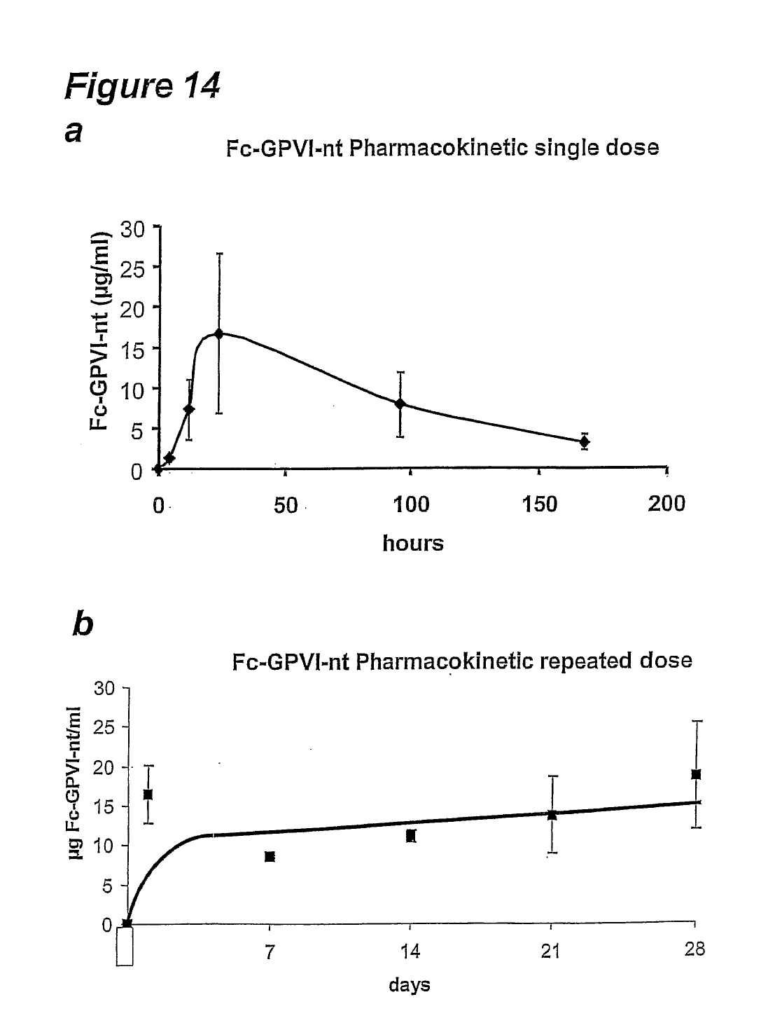

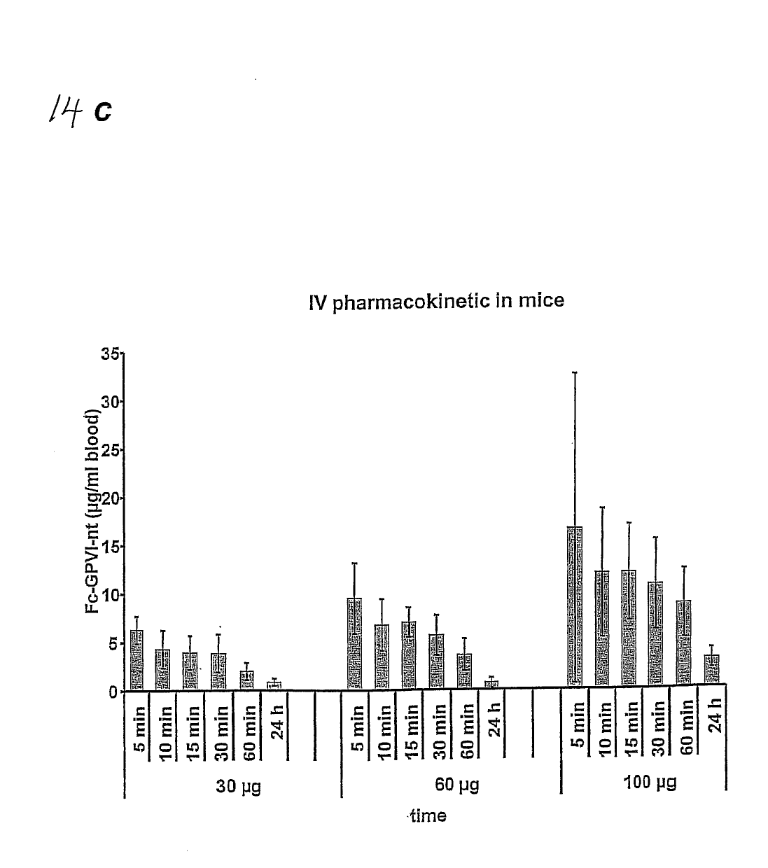

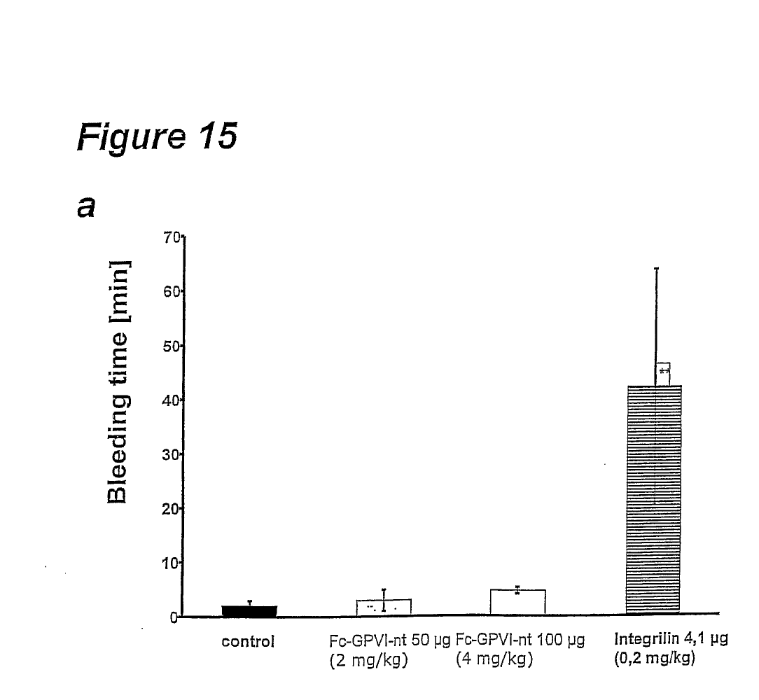

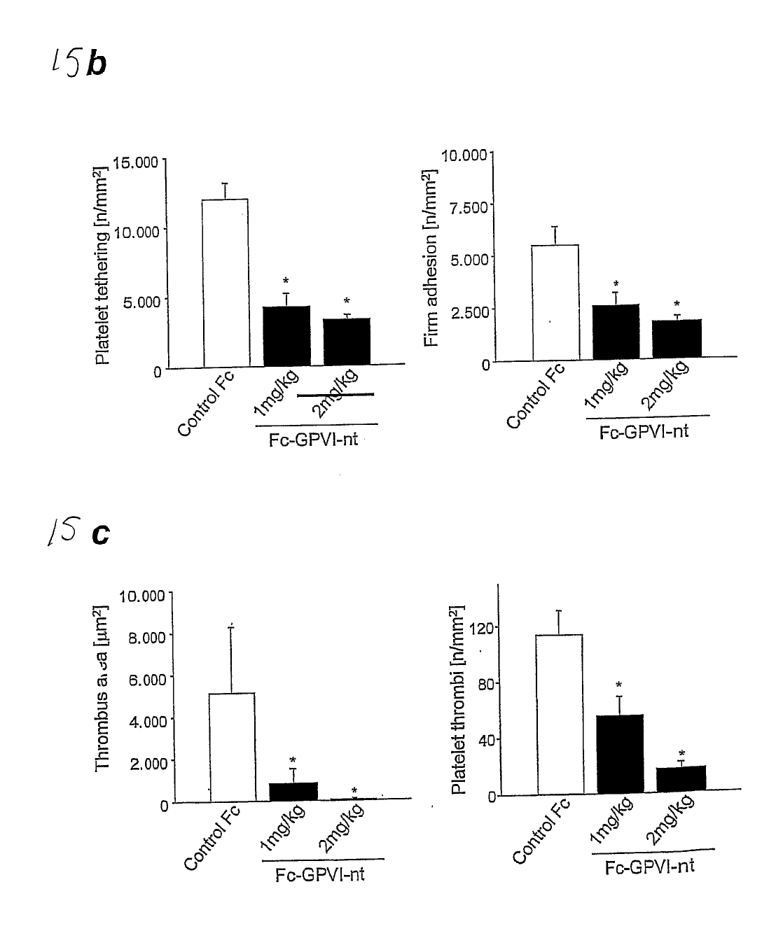

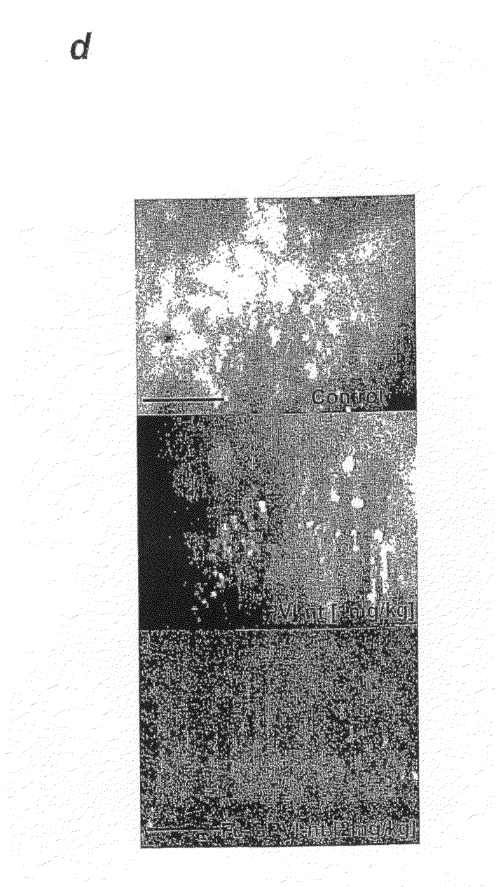

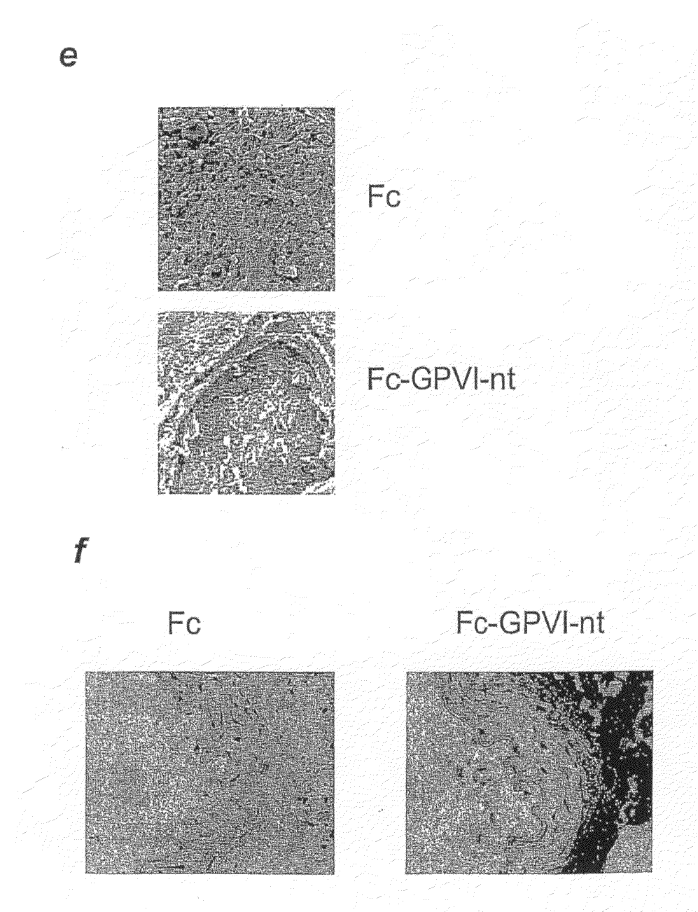

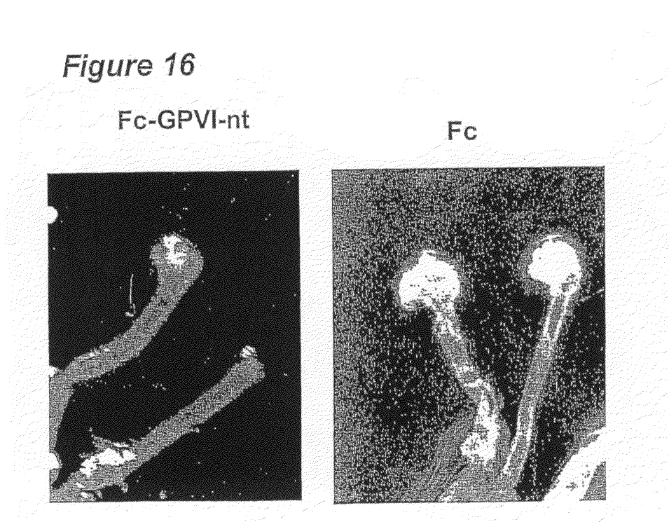

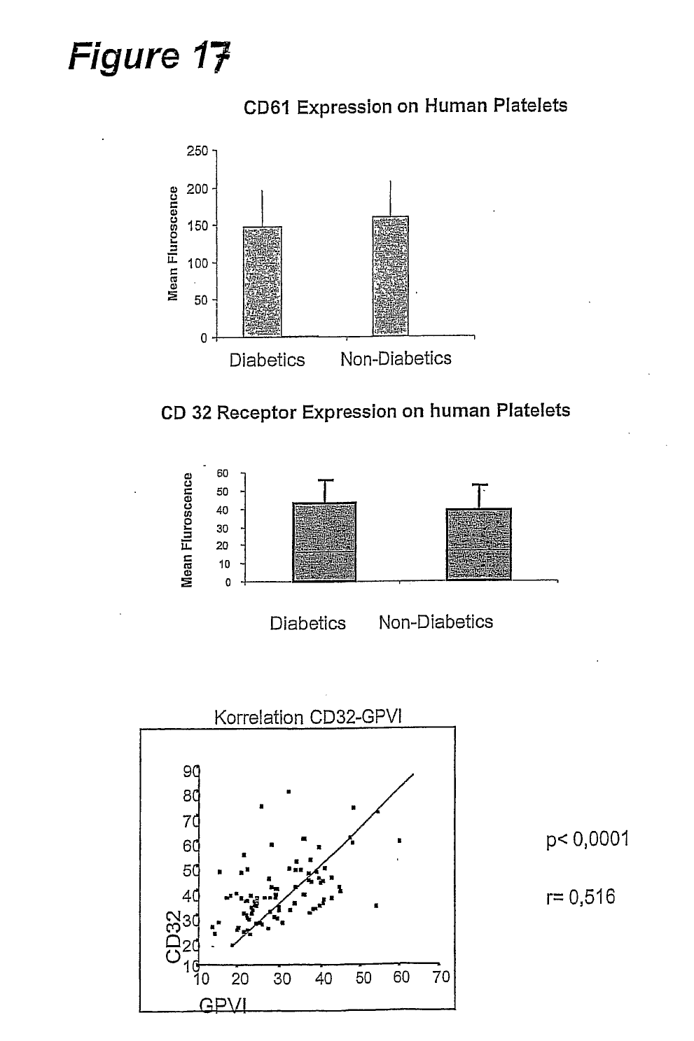

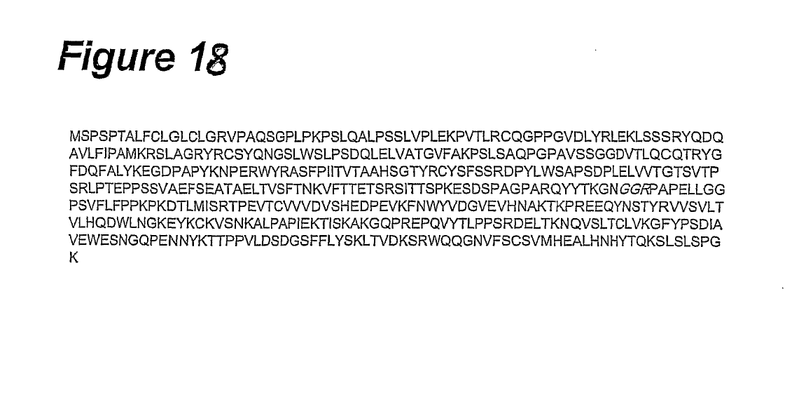

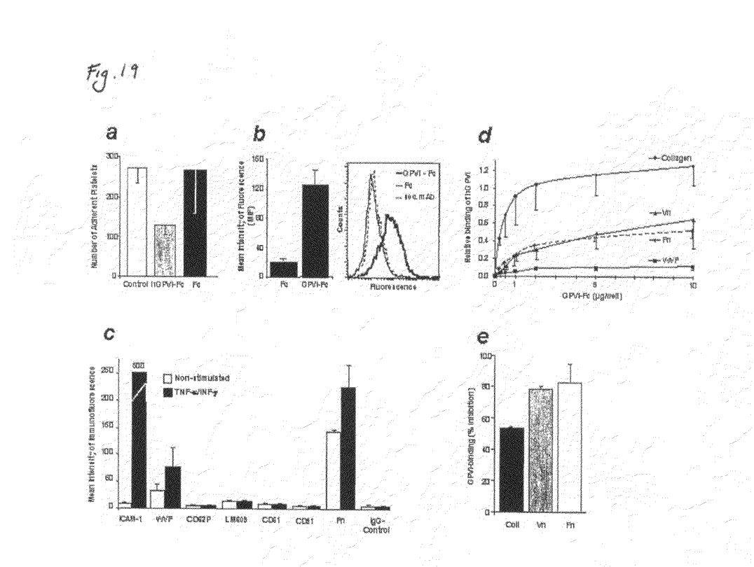

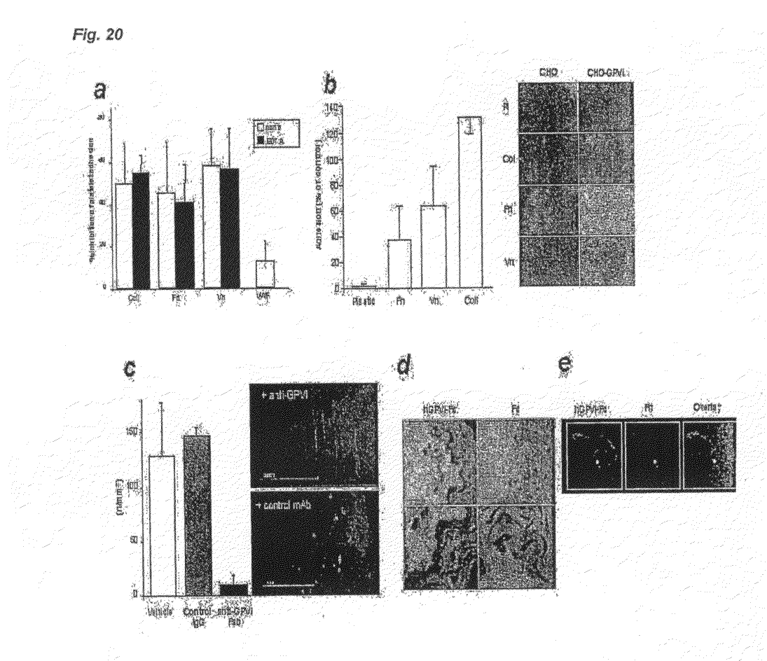

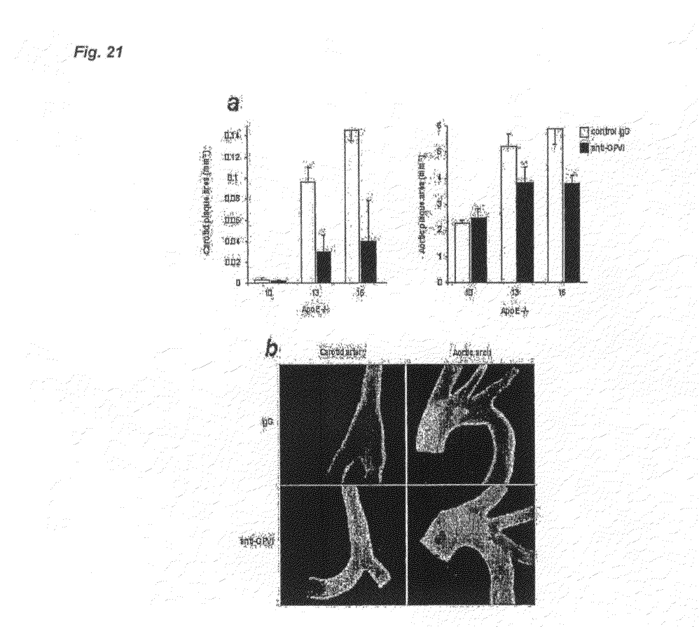

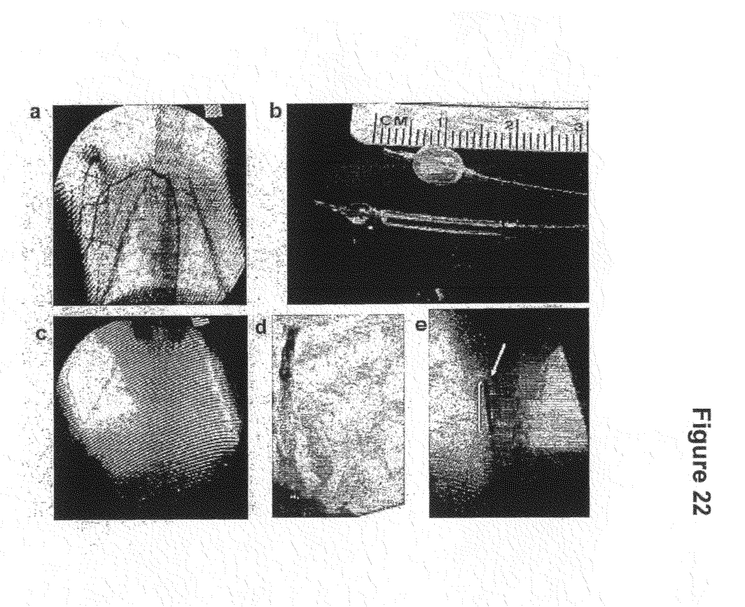

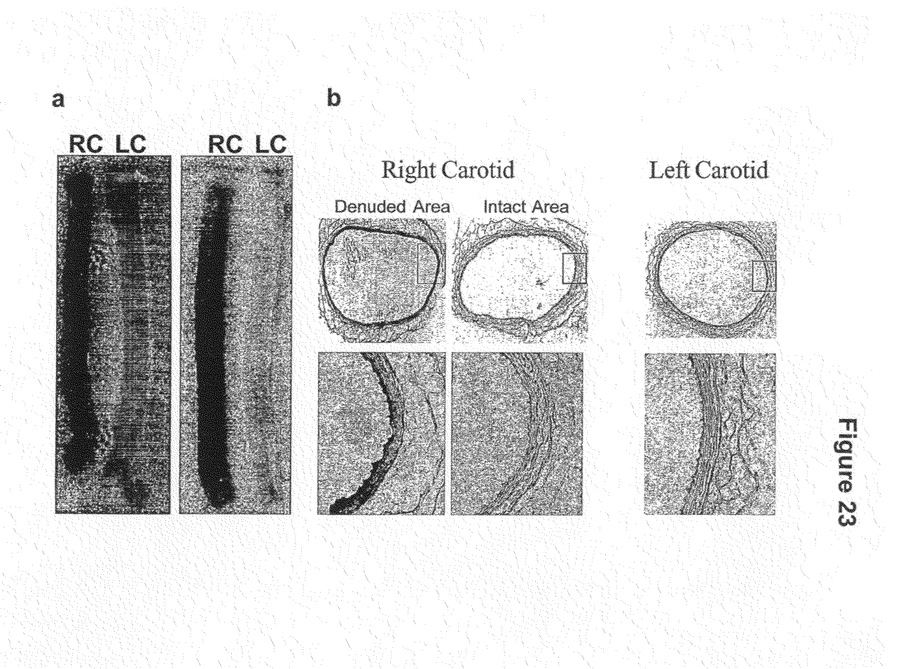

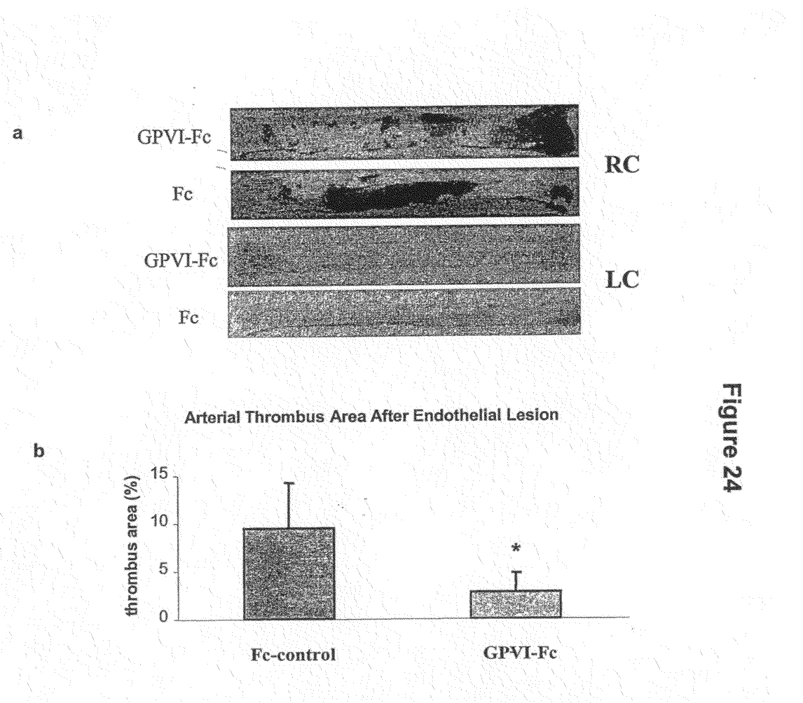

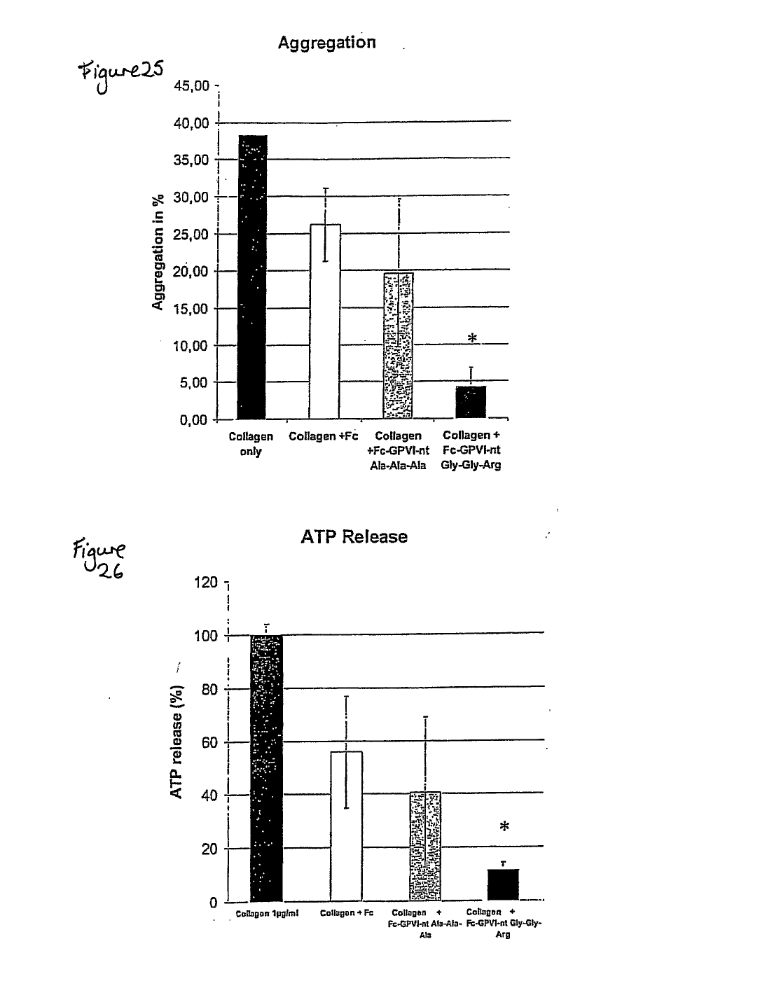

Further to be mentioned are agents, particularly polypeptides, which comprise an amino acid sequence encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8) and which encodes a polypeptide that is capable of binding to a plurality of types of GPVI-binding sites of a dysfunctional or atherosclerotic blood vessel area; or
(ii) a nucleic acid sequence which differs from bases 1 to 807 of SEQ ID No. 2 (FIG. 8) by virtue of the degeneracy of the genetic code.

Also included in the disclosure are agents, particularly polypeptides, comprising an amino acid sequence encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8) and which encodes a polypeptide that is which is capable of inhibiting the binding of GPVI to collagen and to one or both of fibronectin and vitronectin; or
(iii) a nucleic acid sequence which differs from bases 1 to 807 of SEQ ID No. 2 (FIG. 8) by virtue of the degeneracy of the genetic code.

The disclosure includes amongst other things fusion proteins which directly or indirectly inhibit collagen-induced platelet activation and have a longer plasma half-life than the extracellular domain of GPVI as an isolated protein. In this connection, it is known that derivatisation of proteins, e.g. by sugars or by polymers such as, for example, PEG, can increase plasma half-life.

Included are fusion proteins containing an antibody-derived sequence, as in the case of proteins containing at least part of a heavy chain constant region (e.g. at least the hinge) linked to an active sequence (particularly one derived from an extracellular domain of GPVI as mentioned herein) through a linker. Other GPVI fusion proteins, apart from antibody derived fusion proteins, are also encompassed in the present invention.

In one class of proteins the linker comprises the amino acid sequence Gly-X-Z, where Z is a hydrophilic amino acid and X is any amino acid. In some embodiments, X is selected from Gly and Ala. In some embodiments, X may be Gly. In a class of proteins, Z may be selected from Arg, His, Lys, Ser, Thr, Asp, Glu, Tyr, Asn and Gln. In some embodiments, Z may be selected from Arg, Ser, Lys and His: particularly Z is Arg.

The 20 common amino acids may be classified as hydrophobic, polar, positively charged and negatively charged as follows:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=His=histidine
K=Lys=lysine

Negatively Charged Amino Acids
D=Asp=aspartic acid
E=Glu=glutamic acid.

Polar and charged amino acids may be considered hydrophilic, e.g. serine, arginine and lysine. One class of disclosed, proteins comprises a linker having a hydrophilic amino acid selected from positively charged amino acids, e.g. arginine or lysine. A further class comprises a linker having a hydrophilic amino acid selected from negatively charged amino acids. A third class comprises a linker having a hydrophilic amino acid selected from polar uncharged charged amino acids.

The linker may contain additional amino acids to the N-terminal or C-terminal sides of Gly-X-Z, or both sides. Gly is one possible such amino acid. The linker may include the amino acid sequence Gly-Gly-Arg-Gly. Also to be mentioned are linkers comprising a polyglycine amino acid sequence e.g. containing 2, 3, 4, 5 or more contiguous glycine residues. In one embodiment, the linker comprises the amino acid sequence Gly-Arg-Gly.

In one class of proteins, the linker may comprise the amino acid sequence X₁-Gly-Z, where X₁ is not cysteine. X₁ may be Gly or Ala or another amino acid with a hydrophobic side chain, e.g. Val, Ile, Leu, Met, Phe, Pro or Trp. In other embodiments, X₁ is Asp, Gln, Ser, Thr or Tyr. The linker may contain additional amino acids to the N-terminal or C-terminal sides of X₁-Gly-Z, or both sides. Gly is one possible such amino acid.

In one class of proteins, the linker comprises the sequence Gly-Gly-Arg. Gly-Gly-Lys is another linker sequence.

In another class of proteins, the active sequence is at the N-terminal side of the linker and the antibody-derived sequence is at the C-terminal side of the linker. In a sub-class, the linker comprises at least one hydrophilic amino acid. In embodiments, the hydrophilic amino acid may be a residue Z as described above. Some linkers are of the amino acid sequence Gly-X-Z as previously described.

Included are linkers containing at least one of the following sequences as well as 0, 1, 2, 3, 4, 5 or more additional amino acids:

Gly-Gly-Arg;
Gly-Gly-Ser;
Gly-Gly-His;
Gly-Gly-Lys;
Gly-Lys-Gly;
Ser-Gly-Arg;
Arg-Gly-Ser;
Gly-Arg-Arg;
Arg-Gly-Gly;
Gly-Ala-Arg;
Arg-Gly-Arg; and
Arg-Gly-Gly-Ser.

Other linkers may be used in the proteins of the invention. The linker may be flexible.

In one embodiment, the flexible peptide linker is of about 20 or fewer amino acids in length. More preferably, the peptide linker should have at least two amino acids in length. Furthermore, it is even more preferable to use a peptide linker comprising two or more of the following amino acids: glycine, serine, alanine and threonine.

The linker could be at any residue within the extracellular domain of GPVI which would allow the extracellular domain of GPVI to flexibly bind with fibronectin, vitronectin and/or collagen. It will be apparent to one skilled in the art that alternative linkers can be used to link first and second parts. By way of example and by no means of limitation, suitable linkers might be a nucleic acid (egoilgonucleotide); a peptide nucleic acid; a chemical crosslinker (e.g. polyoxyethylene).

Included are fusion proteins which comprise an antibody-derived polypeptide. In one class of protein, the antibody-derived polypeptide includes an Ig heavy chain constant region; in a sub-class of protein, the antibody-derived polypeptide includes a hinge region of an immunoglobulin and is functional to prolong the plasma half-life of the protein beyond that of a fusion protein which does not contain the antibody-derived polypeptide. In another embodiment, the antibody-derived polypeptide includes a hinge region and a CH2 region of an immunoglobulin. In one embodiment, the antibody-derived polypeptide includes a hinge region, a CH₂ and CH3 region of an immunoglobulin. The antibody-derived polypeptide may be an Fc domain of an immunoglobulin, for example. Particular proteins comprise an antibody-derived polypeptide which is an Fc domain of an IgG molecule.

The IgG may be an IgG1. Fc species referred to herein may be IgG derived, particularly IgG1 derived, e.g. derived from such a mouse IgG.

In an alternative embodiment, the antibody-derived polypeptide is a polypeptide which has the properties of an Fc domain of an IgG molecule or a polypeptide which can confer such properties to a fusion protein. Such properties may include, for example, a prolonged serum half-life and thus the incorporation of such a sequence into a fusion protein confers a prolonged serum half-life on the fusion protein as compared to a protein which does not include the antibody-derived polypeptide.

The protein may be conjugated with polyethylene glycol (“PEG”), such conjugation being termed “pegylation” (See Abuchowski et al., J Biol. Chem., 252: 3582-3586 (1977)). PEG is typically characterised as a non-immunogenic uncharged polymer with three water molecules per ethylene oxide monomer. PEG is believed to slow renal clearance by providing increased hydrodynamic volume in pegylated proteins (Maxfield et al, Polymer, 16: 505-509 (1975)). U.S. Pat. No. 5,849,535 also describes humanGH (hGH) variants which are conjugated to one or more polyols, such as poly(ethylene glycol) (PEG).

The protein may be a dimer, for example a dimer containing disulfide bonds between Cys residues of two polypeptides. Typically, the dimer is a homodimer but heterodimers are not excluded. The dimer may be of a polypeptide containing at least part of an Fc hinge region, for example of a polypeptide as described above containing at least a CH2 region and a hinge region, such as CH2, CH3 and hinge regions, for example.

Thus the scope of the disclosure is clearly to be understood to include polypeptides which comprise an antibody derived amino acid sequence linked to an amino acid sequence derived from an extracellular domain of GPVI and which include conservative substitutions, insertions, and/or deletions, but which still retain the biological activity of the fusion protein of FIG. 7 (or SEQ ID No. 1). For example, the number of amino acid residues which are modified, whether by substitution, insert or deletion, may be 1, 2, 3, 4, 5, 6, 7, 8, 9, 10, 11, 12, 13, 14 or 15.

The term “an extracellular domain of GPVI” includes fragments or analogues thereof which have the biological activity of the extracellular domain of GPVI as herein defined. The biological activity of the extracellular domain of GPVI is considered to include, amongst others, collagen binding activity and/or fibronectin binding activity and/or vitronectin binding activity. It will be appreciated that fragments or analogues of GPVI may have greater or less binding affinity to collagen, fibronectin and/or vitronectin than the extracellular domain of GPVI, but nonetheless, will in practice have sufficient binding activity for the protein to be therapeutically useful.

The term “an Fc domain of an immunoglobulin” includes fragments or analogues thereof which have the properties of an Fc domain of an immunoglobulin as herein defined. Alternatively, the IgG Fc may contain amino acid mutations as compared to naturally occurring IgG Fc, for example, to make it non-lytic. In particular embodiments, an Fc is a human Fc; this applies for example to Fc molecules used as a control in certain experiments described herein to assess the activity of PR-15, where an Fc of a human IgG1 may be used.

A particularly advantageous GPVI fusion protein is the protein known as PR-15, which has the amino acid sequence of SEQ ID NO 1 (see FIG. 7). As is described in WO 03/104282, PR-15 is characterised by the linker Gly-Gly-Arg and is therefore a member of a class of a fusion proteins comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

The active, or most active, fusion proteins described in the previous paragraph are dimers thereof. These are capable of binding to a plurality of GPVI-binding sites, optionally a plurality of types of GPVI-binding sites, of a dysfunctional, inflamed or atherosclerotic blood vessel area.

PR-15 is a representative of classes of agents (whether fusion proteins, antibody products, aptamers or otherwise) having one (or more) of the following capabilities shown in Example 13 below:
capable of inhibiting collagen-induced ATP release from human platelets by at least 65%;
capable of inhibiting thrombus formation caused by induction of arterial lesions in the mouse by at least 70% as compared with a control administered with an Fc polypeptide, the relative inhibitions of thrombus formation being determined by measurement of the en face thrombus size as a percentage of an artery wall area placed on a surface for the purpose of said measurement;
capable of inhibiting platelet aggregation in response to collagen by at least 50%.

PR-15 is a representative of classes of agents (whether fusion proteins, antibody products, aptamers or otherwise) having one (or more) of the following characteristics:
1. capable of binding to a plurality of GPVI-binding sites, optionally a plurality of types of GPVI-binding sites, of a dysfunctional, inflamed or atherosclerotic blood vessel area, the agent optionally being capable of inhibiting collagen-induced ATP release from human platelets by at least 65%, e.g. least 70% as in the case of inhibition by at least 80%, e.g. at least 90%;
2. capable of inhibiting GPVI interaction with collagen and a molecule selected from the group consisting of vitronectin, fibronectin and combinations thereof, the agent being capable of inhibiting collagen-induced ATP release from human platelets by at least 65%, e.g. at least 70%, for example 80% or more as in the case of inhibition of at least 90%;
3. capable of binding to a plurality of GPVI-binding sites, optionally a plurality of types of GPVI-binding sites, of a dysfunctional, inflamed or atherosclerotic blood vessel area, the agent being capable of inhibiting thrombus formation caused by induction of arterial lesions in the mouse by at least 70% as compared with a control administered with an Fc polypeptide, the relative inhibitions of thrombus formation being determined by measurement of the en face thrombus size as a percentage of an artery wall area placed on a surface for the purpose of said measurement, the inhibition optionally being by at least 80%, as in the case of inhibition of the thrombus formation by at least 90%, e.g. at least 95%;
4. capable of inhibiting GPVI interaction with collagen and a molecule selected from the group consisting of vitronectin, fibronectin and combinations thereof, the agent being capable of inhibiting thrombus formation caused by induction of arterial lesions in the mouse by at least 70%, e.g. at least 80% and for example at least 90%, e.g. at least 95%, as compared with a control not administered with an Fc polypeptide, the relative inhibitions of thrombus formation being determined by measurement of the en face thrombus size as a percentage of an artery wall area placed on a surface for the purpose of said measurement;
5. capable of binding to a plurality of GPVI-binding sites, optionally a plurality of types of GPVI-binding sites, of a dysfunctional, inflamed or atherosclerotic blood vessel area, the agent being capable of inhibiting platelet aggregation in response to collagen by at least 50%, optionally by at least 60%, for example at least 70%, e.g. 75% or more;
6. capable of inhibiting binding of GPVI to collagen and a molecule selected from the group consisting of vitronectin, fibronectin and combinations thereof, the agent being capable of inhibiting platelet aggregation in response to collagen by at least 50%, e.g. at least 60%, for example at least 70%, e.g. 75% or more.

Amongst the embodiments of the disclosure are those in which PR-15 and members of the above mentioned classes of agent to which PR-15 belongs are used to interfere with or inhibit an interaction with one of both of fibronectin and vitronectin. Examples of such uses are prophylaxis against adhesion of platelets and/or monocytes to blood vessels in patients asymptomatic for atherosclerosis and/or thrombosis, and in patients considered at risk of suffering atherosclerosis and/or cardiovascular disease.

Other embodiments are those in which an agent is used to interfere with or inhibit an interaction with collagen; in sub-embodiments, the agent is not PR-15 or another member of the class of a fusion proteins comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

In fact, all the uses, methods and patient classes described herein may be applied to (i) agents which are fusion proteins as described in the preceding paragraph; (ii) agents which are not fusion proteins as described in the preceding paragraph; (iii) agents which are GPVI fusion proteins; (iv) agents which are not GPVI fusion proteins; (v) agents which do not comprise a GPVI-derived sequence; (vi) agents which do comprise a GPVI-derived sequence; (vii) agents which are antibody products (e.g. antibodies or antibody fragments); (viii) agents which are aptamers; (ix) agents which comprise a sequence derived from a snake venom; (x) agents which do fall within any one or more of paragraphs A1 to A105 below; (xi) agents which do not fall within any one or more of paragraphs A1 to A105 below.

In a further aspect of the present disclosure, there is provided a nucleic acid comprising a nucleic acid sequence, which sequence encodes an agent as described herein when the agent is a polypeptide product, for example an antibody, a fragment thereof, a fusion protein, or a protein.

The present disclosure includes a nucleic acid sequence which encodes an agent comprising a wild type amino acid sequence but whose coding sequence for said wild type amino acid sequence differs from the wild-type nucleic acid sequence as a result of the degeneracy of the genetic code.

The present disclosure also includes nucleic acids that share at least 90% homology with a nucleic acid sequence which encodes an agent of the present disclosure. In particular, the nucleic add may have 90%, 91%, 92%, 93%, 94%, 95%, 96% 97%, 98% or 99% homology to such a nucleic acid. Included are such nucleic acids wherein the agent has a wild type sequence, and polypeptides encoded by such nucleic acids.

In one aspect, there is provided a nucleic acid molecule which hybridises under stringent conditions to a nucleic acid molecule which encodes an agent of the disclosure.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following have been found as exemplary for hybridization conditions but without limitation:

Very High Stringency (allows sequences that
share at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share
at least 80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share
at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

A common formula for calculating the stringency conditions required to achieve hybridisation between nucleic acid molecules of a specified homology is:

Tₘ=81.5° C.+16.6 Log [Na⁺]+0.41[% G+C]−0.63(% formamide)

Also included are nucleic acid sequences which differ from those described above by virtue of the degeneracy of the genetic code.

One aspect of the disclosure pertains to isolated nucleic acid molecules (e.g., cDNAs) comprising a nucleotide sequence encoding a fusion protein comprising an extracellular domain of GPVI and an Fc domain of an immunoglobulin. In particular embodiments, the isolated nucleic acid molecule comprises one of the nucleotide sequences set forth in Sequence SEQ ID NO: 2 (FIG. 8) or the coding region or a complement thereof. In other embodiments, the isolated nucleic acid molecule comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence as in SEQ ID NO: 2 (FIG. 8), or a portion thereof. In other preferred embodiments, the isolated nucleic acid molecule encodes one of the amino acid sequences set forth in SEQ ID NO: 1 (FIG. 7).

In a related aspect, an expression vector is provided which comprises a nucleic acid as described herein and associated regulatory sequences necessary for expression of a protein or polypeptide in a host cell. As used herein, a “vector” may be any of a number of nucleic acids into which a desired sequence may be inserted. Vectors include, but are not limited to, plasmids, phagemids and virus genomes. In a further embodiment said viral based vector is based on viruses selected from the group consisting of adenovirus; retrovirus; adeno associated virus; herpesvirus; lentivirus; baculovirus.

Further to be mentioned are host cells comprising a nucleic acid as described herein or a vector as described herein. Such host cells are transfected or transformed so that they contain the nucleic acid or vector in such a way that they are effective in expressing the desired polypeptide/protein when cultured in appropriate media under the necessary growth conditions. In one embodiment the host cell is selected from a HeLa cell and a CHO (Chinese Hamster Ovary) cell. The host cells to be used are not particularly circumscribed so as long as they can be transfected by a vector to be used and can express the DNA of the present disclosure. For example, bacteria such as Escherichia coli, yeast such as Saccharomyces cerevisiae, and an animal cell such as a COS cell, a CHO cell, etc. can be used.

Examples of prokaryotic host cells include E. coli. Examples of eukaryotic host cells include avian, insect, plant, and animal cells such as COS7, HeLa, and CHO cells.

By cultivating a transformant or transfected cell, a polypeptide agent, for example a fusion protein, antibody or antibody fragment, can be produced in a cell or a culture medium. Then, by collecting the produced polypeptide, e.g. an antibody (or antibody fragment), a polypeptidic agent can be obtained. The obtained polypeptide e.g. antibody or protein can be isolated and purified by appropriately combining methods, for example, centrifugation, ammonium sulfate fractionation, salting out, ultrafiltration, affinity chromatography, ion-exchange chromatography, or gel-filtration chromatography.

For example, the cells can be cultured in a suitable medium, and spent medium can be used as an polypeptide source. Optionally, matrix-coated channels or beads and cell cocultures may be included to enhance growth of transformed cells e.g. antibody-producing cells.

For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal is optionally primed for ascites production by prior administration of a suitable composition, for example, Pristane. Antibodies may also be obtained by employing routine recombinant methods such as described in Sambrook et al. (1989) supra. For Instance, nucleic acid sequences of the disclosure can be cloned into a suitable expression vector (which contains control sequences for transcription, such as a promoter). The expression vector is in turn introduced into a host cell. The host cell is grown under suitable conditions such that the polynucleotide is transcribed and translated into a protein. Heavy and light chains of antibodies may be produced separately, and then combined by disulfide bond rearrangement. Alternatively, vectors with separate polynucleotides encoding each chain of an antibody of the disclosure, or a vector with a single polynucleotide encoding both chains as separate transcripts, may be transfected into a single host cell which may then produce and assemble the entire molecule. Preferably, the host cell is a higher eukaryotic cell that can provide the normal carbohydrate complement of the molecule. The antibody is thus produced in the host cell can be purified using standard techniques in the art.

The agents of this disclosure can be made by any suitable procedure, including by recombinant methods or by chemical synthesis, as appropriate. Peptides which are produced may then be separated from each other by techniques known in the art, including but not limited to, gel filtration chromatography, gel electrophoresis, and reverse-phase HPLC.

Alternatively, polypeptidic agents of the disclosure can be chemically synthesized using information provided in this disclosure, in conjunction with standard methods of protein synthesis. A suitable method is the solid-phase Merrifield technique. Automated peptide synthesizers are commercially available, such as those manufactured by Applied Biosystems, Inc. (Foster City, Calif.).

The disclosure further includes the subject matter of the following paragraphs:

A1. A fusion protein comprising:
a) a first polypeptide which is capable of inhibiting adhesion of platelets to collagen;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A2. The fusion protein of Paragraph A1, wherein the first polypeptide is capable of binding to collagen.

A3. The fusion protein of Paragraph A1 or Paragraph A2, wherein the first polypeptide comprises an extracellular domain of GPVI or a variant thereof that is functional for binding to collagen.

A4. The fusion protein of any preceding Paragraph (i.e. Paragraph A1, A2 or A3), wherein the first polypeptide binds to collagen competitively with platelet-bound GPVI.

A5. The fusion protein of any preceding Paragraph, wherein the first polypeptide is functional for binding to collagen at the platelet-bound GPVI binding site of collagen.

A6. The fusion protein of any preceding Paragraph, wherein the second polypeptide comprises an amino acid sequence of an Ig heavy chain constant part.

A7. The fusion protein of any preceding Paragraph, wherein the second polypeptide comprises a hinge region of an immunoglobulin and is functional to prolong the plasma half-life beyond that of a protein consisting of the first polypeptide and the linker.

A8. The fusion protein of any preceding Paragraph, wherein the second polypeptide comprises a hinge region and a CH2 region of an immunoglobulin.

A9. The fusion protein of any preceding Paragraph, wherein the second polypeptide comprises a hinge region, a CH2 region and a CH3 region of an immunoglobulin.

A10. The fusion protein of any preceding Paragraph, wherein the first polypeptide comprises an extracellular domain of GPVI or a variant thereof that is functional for binding to collagen; and the second polypeptide comprises an Fc domain of an immunoglobulin or a functional conservative part thereof.

A11. The fusion protein of any preceding Paragraph wherein Z is selected from the group consisting of Arg, Ser, Thr, Asp, Glu, Tyr, Asn and Gln.

A12. The fusion protein of any preceding Paragraph, wherein Z is Ser.

A13. The fusion protein of any preceding Paragraph, wherein Z is Arg.

A14. The fusion protein of any preceding Paragraph, wherein the linker comprises the amino acid sequence Gly-X-Z and X is Gly.

A15. The fusion protein of any preceding Paragraph, wherein P is Gly.

A16. The fusion protein of any preceding Paragraph, wherein the linker comprises a diglycine sequence.

A17. The fusion protein of any preceding Paragraph, which is expressed in a mammalian cell.

A18. A fusion protein comprising:
a) an amino acid sequence which, when the protein is administered, results in inhibition of adhesion of platelets to collagen; and
b) an antibody-derived amino acid sequence, wherein sequence (a) is linked at its C-terminus to the N-terminus of sequence (b) through a linker comprising a hydrophilic amino acid.

A19. The fusion protein of Paragraph A18, wherein the antibody-derived amino acid sequence is an Fc domain of an immunoglobulin.

A20. The fusion protein of Paragraph A18 or Paragraph A19, wherein the amino acid sequence which results in the inhibition of adhesion of platelets to collagen is an extracellular domain of GPVI.

A21. The fusion protein of any of Paragraphs A18 to A20, wherein the hydrophilic amino acid is selected from Arg, Ser, Thr, Lys, His, Glu, Asp and Asn.

A22. The fusion protein of any of Paragraphs A18 to A21, wherein the hydrophilic amino acid is Arg.

A23. A fusion protein comprising:
a) an amino acid sequence comprising an extracellular domain of GPVI or a variant thereof that is functional for binding to collagen;
b) a linker comprising an amino acid sequence Gly-Gly-Z, wherein Z is a hydrophilic amino acid; and
c) an amino acid sequence comprising an Fc domain of an immunoglobulin or a functional conservative part thereof.

A24. The fusion protein of Paragraph A23, wherein the amino acid sequence (a) is encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8); or
(iii) a nucleic acid sequence which differs from bases 1 to 807 of SEQ ID No. 2 (FIG. 8) by virtue of the degeneracy of the genetic code.

A25. The fusion protein of Paragraph A23 or Paragraph A24, wherein amino acid sequence (b) is encoded by:
(i) a nucleic acid sequence of bases 808 to 816 of SEQ ID. No 2 (FIG. 8);
(ii) a nucleic acid sequence which hybridises to bases 808 to 816 of SEQ ID No. 2 (FIG. 8);
(iii) a nucleic acid sequence which differs from bases 808 to 816 of SEQ ID No. 2 (FIG. 8) by virtue of the degeneracy of the genetic code

A26. The fusion protein of any of Paragraphs A23 to A25, wherein the hydrophilic amino acid is selected from Arg, His and Lys.

A27. The fusion protein of any of Paragraphs A23 to A26, wherein amino acid sequence (c) is encoded by:
(i) a nucleic acid sequence of bases 817 to 1515 of SEQ ID. No. 2 (FIG. 8);
(ii) a nucleic acid sequence which hybridises to bases 817 to 1515 of SEQ ID No. 2 (FIG. 8); or
(iii) a nucleic acid sequence which differs from bases 817 to 1515 of SEQ ID No. 2 (FIG. 8) by virtue of the degeneracy of the genetic code.

A28. A protein having the characteristics of a protein obtained by expressing in a mammalian cell under non-reducing conditions a DNA sequence comprising in a 5′ to 3′ direction:
(i) a DNA sequence comprising bases 1 to 807 of SEQ ID No. 2 (FIG. 8) or a variant thereof in which said sequence encoded by bases 1 to 807 of SEQ ID No. 2 (FIG. 8) is replaced by a variant having collagen binding activity;
(ii) a DNA sequence comprising bases 808 to 816 of SEQ ID. No. 2 (FIG. 8); and
(iii) a DNA sequence comprising bases 817 to 1515 of SEQ ID No. 2 (FIG. 8) or a variant thereof in which said sequence encoded by bases 817 to 1515 of SEQ ID No. 2 (FIG. 8) is replaced by a variant having the properties of an Fc region.

A29. A dimer of a polypeptide, the polypeptide comprising
a) an extracellular domain of GPVI or a variant thereof that is functional for binding to collagen; and
b) a linker comprising an amino acid sequence Gly-Gly-Z, wherein Z is a hydrophilic amino acid; and
c) an Fc domain of an immunoglobulin or a functional conservative part thereof.

A30. A method of treating or preventing thrombosis in a subject, comprising administering to the subject a therapeutically effective amount of a fusion protein comprising:
a) an amino acid sequence which, when the protein is administered, results in inhibition of collagen-induced platelet activation; and
b) a antibody-derived amino acid sequence, wherein sequence (a) is linked at its C-terminus to the N-terminus of sequence (b) through a linker comprising a hydrophilic amino acid.

A31. The method of Paragraph 30, wherein amino acid sequence (a) comprises an extracellular domain of GPVI or a variant thereof that is functional for binding to collagen; and the antibody-derived amino acid sequence comprises an Fc domain of an immunoglobulin or a functional conservative part thereof.

A32. The method of Paragraph A30 or Paragraph A31, wherein the fusion protein comprises sequentially in an N-terminus to C-terminus direction, a first amino acid sequence, a second amino acid sequence and a third amino acid sequence wherein said first amino acid sequence comprises:
A) i) an amino acid sequence encoded by a nucleic acid sequence of bases 1 to 807 of SEQ ID No. 2 (FIG. 8);
   ii) an amino acid sequence encoded by a nucleic acid sequence which hybridises to bases 1 to 807 of SEQ ID No. 2 (FIG. 8) and which codes for a polypeptide which binds to collagen; or
   iii) an amino acid sequence encoded by a nucleic add sequence which differs from bases 1 to 807 of SEQ ID No. 2 (FIG. 8) by virtue of the degeneracy of the genetic code and which binds to collagen;
      and wherein the second amino acid comprises:
B) i) an amino acid sequence encoded by a nucleic acid sequence of bases 808 to 816 of SEQ ID No. 2 (FIG. 8);
   ii) a 3-mer amino acid sequence containing a hydrophilic amino acid or
   iii) an amino acid sequence encoded by a nucleic acid sequence which differs from bases 808 to 816 of SEQ ID No. 2 (FIG. 8) by virtue of the degeneracy of the genetic code;
      wherein the third amino acid sequence comprises:
C) i) an amino acid sequence encoded by a nucleic acid sequence of bases 817 to 1515 of SEQ ID. No. 2 (FIG. 8);
   ii) an amino acid sequence encoded by a nucleic acid sequence which hybridises to bases 817 to 1515 of SEQ ID No. 2 (FIG. 8) and which codes for a polypeptide which is functional as an Fc domain of an immunoglobulin; or
   iii) an amino acid sequence encoded by a nucleic acid sequence which differs from bases 817 to 1515 of SEQ ID No. 2 (FIG. 8) by virtue of the degeneracy of the genetic code and which is functional as an Fc domain of an immunoglobulin.

A33. A dimer of a polypeptide, the polypeptide comprising:
a) a first polypeptide which is capable of inhibiting adhesion of platelets to collagen;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A34. A dimer of a polypeptide, the polypeptide comprising:
a) a first polypeptide which is capable of inhibiting adhesion of platelets to collagen;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid,
   wherein the first polypeptide binds to collagen competitively with platelet-bound GPVI, the second polypeptide comprises a hinge region of an immunoglobulin and is functional to prolong the plasma half-life beyond that of a protein consisting of the first polypeptide and the linker and wherein the linker comprises the amino acid sequence Gly-X-Z, wherein Z is selected from the group consisting of Arg, Ser, Thr, Asp, Glu, Tyr, Asn and Gln.

A35. A dimer of a polypeptide, the polypeptide comprising:
a) a first polypeptide which is capable of inhibiting adhesion of platelets to collagen;
b) a second, antibody-derived polypeptide; and
c) a linker comprising an amino acid sequence Gly-X-Z, wherein X is Gly and Z is selected from the group consisting of Lys and Arg,
   wherein the first polypeptide binds to collagen competitively with platelet-bound GPVI and wherein the second polypeptide comprises a hinge region of an immunoglobulin and is functional to prolong the plasma half-life beyond that of a protein consisting of the first polypeptide and the linker.

A36. A fusion protein comprising:
a) a first polypeptide which is capable of inhibiting adhesion of platelets to fibronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A37. The fusion protein of Paragraph A36, wherein the first polypeptide is capable of binding to fibronectin.

A38. The fusion protein of Paragraph A37 or Paragraph A38, wherein the first polypeptide comprises an extracellular domain of GPVI or a variant thereof that is functional for binding to fibronectin.

A39. The fusion protein of any of Paragraphs A36 to A38, wherein the first polypeptide binds to fibronectin competitively with platelet-bound GPVI.

A40. The fusion protein of any of Paragraphs A36 to A39, wherein the first polypeptide is functional for binding to fibronectin at the platelet-bound GPVI binding site of fibronectin.

A41. The fusion protein of any of Paragraphs A36 to A40, wherein the second polypeptide comprises an amino acid sequence of an Ig heavy chain constant region.

A42. The fusion protein of any of Paragraphs A36 to A41, wherein the second polypeptide comprises a hinge region of an immunoglobulin and is functional to prolong the plasma half-life beyond that of a protein consisting of the first polypeptide and the linker.

A43. The fusion protein of any of Paragraphs A36 to A42, wherein the second polypeptide comprises a hinge region and a CH2 region of an immunoglobulin.

A44. The fusion protein of any of Paragraphs A36 to A43, wherein the second polypeptide comprises a hinge region, a CH2 region and a CH3 region of an immunoglobulin.

A45. The fusion protein of any of Paragraphs A36 to A44, wherein the first polypeptide comprises an extracellular domain of GPVI or a variant thereof that is functional for binding to fibronectin; and the second polypeptide comprises an Fc domain of an immunoglobulin or a functional conservative part thereof.

A46. The fusion protein of any of Paragraphs A36 to A45 wherein Z is selected from the group consisting of Arg, Ser, Thr, Asp, Glu, Tyr, Asn and Gln.

A47. The fusion protein of any of Paragraphs A36 to A46, wherein Z is Ser.

A48. The fusion protein of any of Paragraphs A36 to A47, wherein Z is Arg.

A49. The fusion protein of any of Paragraphs A36 to A48, wherein the linker comprises the amino acid sequence Gly-X-Z and X is Gly.

A50. The fusion protein of any of Paragraphs A36 to A49, wherein P is Gly.

A51. The fusion protein of any of Paragraphs 36 to 50, wherein the linker comprises a diglycine sequence.

A52. The fusion protein of any of Paragraphs A36 to A51, which is expressed in a mammalian cell.

A53. A fusion protein comprising:
a) an amino acid sequence which, when the protein is administered, results in inhibition of adhesion of platelets to fibronectin; and
b) an antibody-derived amino acid sequence,
   wherein sequence (a) is linked at its C-terminus to the N-terminus of sequence (b) through a linker comprising a hydrophilic amino acid.

A54. The fusion protein of Paragraph A53, wherein the antibody-derived amino acid sequence is an Fc domain of an immunoglobulin.

A55. The fusion protein of Paragraph A53 or Paragraph A54, wherein the amino acid sequence which results in the inhibition of adhesion of platelets to fibronectin is an extracellular domain of GPVI.

A56. The fusion protein of any of Paragraphs A53 to A55, wherein the hydrophilic amino acid is selected from Arg, Ser, Thr, Lys, His, Glu, Asp and Asn.

A57. The fusion protein of any of Paragraphs A53 to A56, wherein the hydrophilic amino acid is Arg.

A58. A fusion protein comprising:
a) an amino acid sequence comprising an extracellular domain of GPVI or a variant thereof that is functional for binding to fibronectin;
b) a linker comprising an amino acid sequence Gly-Gly-Z, wherein Z is a hydrophilic amino acid; and
c) an amino acid sequence comprising an Fc domain of an immunoglobulin or a functional conservative part thereof.

A59. The fusion protein of Paragraph A58, wherein the amino acid sequence (a) is encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8); or
(iii) a nucleic acid sequence which differs from bases 1 to 807 of SEQ ID No. 2 (FIG. 8) by virtue of the degeneracy of the genetic code.

A60. The fusion protein of Paragraph A58 or Paragraph A59, wherein amino acid sequence (b) is encoded by:
(i) a nucleic acid sequence of bases 808 to 816 of SEQ ID. No 2 (FIG. 8);
(ii) a nucleic acid sequence which hybridises to bases 808 to 816 of SEQ ID No. 2 (FIG. 8);
(iii) a nucleic acid sequence which differs from bases 808 to 816 of SEQ ID No. 2 (FIG. 8) by virtue of the degeneracy of the genetic code

A61. The fusion protein of any of Paragraphs A58 to A60, wherein the hydrophilic amino acid is selected from Arg, His and Lys.

A62. The fusion protein of any of Paragraphs A58 to A61, wherein amino acid sequence (c) is encoded by:
(i) a nucleic acid sequence of bases 817 to 1515 of SEQ ID. No. 2 (FIG. 8);
(ii) a nucleic acid sequence which hybridises to bases 817 to 1515 of SEQ ID No. 2 (FIG. 8); or
(iii) a nucleic acid sequence which differs from bases 817 to 1515 of SEQ ID No. 2 (FIG. 8) by virtue of the degeneracy of the genetic code.

A63. A protein having the characteristics of a protein obtained by expressing in a mammalian cell under non-reducing conditions a DNA sequence comprising in a 5′ to 3′ direction:
(i) a DNA sequence comprising bases 1 to 807 of SEQ ID No. 2 (FIG. 8) or a variant thereof in which said sequence encoded by bases 1 to 807 of SEQ ID No. 2 (FIG. 8) is replaced by a variant having fibronectin binding activity;
(ii) a DNA sequence comprising bases 808 to 816 of SEQ ID. No. 2 (FIG. 8); and
(iii) a DNA sequence comprising bases 817 to 1515 of SEQ ID No. 2 (FIG. 8) or a variant thereof in which said sequence encoded by bases 817 to 1515 of SEQ ID No. 2 (FIG. 8) is replaced by a variant having the properties of an Fc region.

A64. A dimer of a polypeptide, the polypeptide comprising
a) an extracellular domain of GPVI or a variant thereof that is functional for binding to fibronectin; and
b) a linker comprising an amino acid sequence Gly-Gly-Z, wherein Z is a hydrophilic amino acid; and
c) an Fc domain of an immunoglobulin or a functional conservative part thereof.

A65. A method of treating or preventing thrombosis in a subject, comprising administering to the subject a therapeutically effective amount of a fusion protein comprising:
a) an amino acid sequence which, when the protein is administered, results in inhibition of fibronectin-induced platelet activation; and
b) a antibody-derived amino acid sequence, wherein sequence (a) is linked at its C-terminus to the N-terminus of sequence (b) through a linker comprising a hydrophilic amino acid.

A66. The method of Paragraph A65, wherein amino acid sequence (a) comprises an extracellular domain of GPVI or a variant thereof that is functional for binding to fibronectin; and the antibody-derived amino acid sequence comprises an Fc domain of an immunoglobulin or a functional conservative part thereof.

A67. The method of Paragraph A65 or Paragraph A66, wherein the fusion protein comprises sequentially in an N-terminus to C-terminus direction, a first amino acid sequence, a second amino acid sequence and a third amino acid sequence wherein said first amino acid sequence comprises:
A) i) an amino acid sequence encoded by a nucleic acid sequence of bases 1 to 807 of SEQ ID No. 2 (FIG. 8);
   ii) an amino acid sequence encoded by a nucleic acid sequence which hybridises to bases 1 to 807 of SEQ ID No. 2 (FIG. 8) and which codes for a polypeptide which binds to fibronectin; or
   iii) an amino acid sequence encoded by a nucleic acid sequence which differs from bases 1 to 807 of SEQ ID No. 2 (FIG. 8) by virtue of the degeneracy of the genetic code and which binds to fibronectin;
      and wherein the second amino acid comprises
B) i) an amino acid sequence encoded by a nucleic acid sequence of bases 808 to 816 of SEQ ID No. 2 (FIG. 8);
   ii) a 3-mer amino acid sequence containing a hydrophilic amino acid or
   iii) an amino acid sequence encoded by a nucleic acid sequence which differs from bases 808 to 816 of SEQ ID No. 2 (FIG. 8) by virtue of the degeneracy of the genetic code;
      wherein the third amino acid sequence comprises:
C) i) an amino acid sequence encoded by a nucleic acid sequence of bases 817 to 1515 of SEQ ID. No. 2 (FIG. 8);
   ii) an amino acid sequence encoded by a nucleic acid sequence which hybridises to bases 817 to 1515 of SEQ ID No. 2 (FIG. 8) and which codes for a polypeptide which is functional as an Fc domain of an immunoglobulin; or
   iii) an amino acid sequence encoded by a nucleic acid sequence which differs from bases 817 to 1515 of SEQ ID No. 2 (FIG. 8) by virtue of the degeneracy of the genetic code and which is functional as an Fc domain of an immunoglobulin.

A68. A dimer of a polypeptide, the polypeptide comprising:
a) a first polypeptide which is capable of inhibiting adhesion of platelets to fibronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A69. A dimer of a polypeptide, the polypeptide comprising:
a) a first polypeptide which is capable of inhibiting adhesion of platelets to fibronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid,
   wherein the first polypeptide binds to fibronectin competitively with platelet-bound GPVI, the second polypeptide comprises a hinge region of an immunoglobulin and is functional to prolong the plasma half-life beyond that of a protein consisting of the first polypeptide and the linker and wherein the linker comprises the amino acid sequence Gly-X-Z, wherein Z is selected from the group consisting of Arg, Ser, Thr, Asp, Glu, Tyr, Asn and Gln.

A70. A dimer of a polypeptide, the polypeptide comprising:
a) a first polypeptide which is capable of inhibiting adhesion of platelets to fibronectin;
b) a second, antibody-derived polypeptide; and
c) a linker comprising an amino acid sequence Gly-X-Z, wherein X is Gly and Z is selected from the group consisting of Lys and Arg,
   wherein the first polypeptide binds to fibronectin competitively with platelet-bound GPVI and wherein the second polypeptide comprises a hinge region of an immunoglobulin and is functional to prolong the plasma half-life beyond that of a protein consisting of the first polypeptide and the linker.

A71. A fusion protein comprising:
a) a first polypeptide which is capable of inhibiting adhesion of platelets to vitronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A72. The fusion protein of Paragraph A71, wherein the first polypeptide is capable of binding to vitronectin.

A73. The fusion protein of Paragraph A71 or Paragraph A72, wherein the first polypeptide comprises an extracellular domain of GPVI or a variant thereof that is functional for binding to vitronectin.

A74. The fusion protein of any of Paragraphs A71 to A73, wherein the first polypeptide binds to vitronectin competitively with platelet-bound GPVI.

A75. The fusion protein of any of Paragraphs A71 to A73, wherein the first polypeptide is functional for binding to vitronectin at the platelet-bound GPVI binding site of vitronectin.

A76. The fusion protein of any of Paragraphs A71 to A74, wherein the second polypeptide comprises an amino acid sequence of an Ig heavy chain constant region.

A77. The fusion protein of any of Paragraphs A71 to A75, wherein the second polypeptide comprises a hinge region of an immunoglobulin and is functional to prolong the plasma half-life beyond that of a protein consisting of the first polypeptide and the linker.

A78. The fusion protein of any of Paragraphs A71 to A76, wherein the second polypeptide comprises a hinge region and a CH2 region of an immunoglobulin.

A79. The fusion protein of any of Paragraphs A71 to A78, wherein the second polypeptide comprises a hinge region, a CH2 region and a CH3 region of an immunoglobulin.

A80. The fusion protein of any of Paragraphs A71 to A79, wherein the first polypeptide comprises an extracellular domain of GPVI or a variant thereof that is functional for binding to vitronectin; and the second polypeptide comprises an Fc domain of an immunoglobulin or a functional conservative part thereof.

A81. The fusion protein of any of Paragraphs A71 to A80 wherein Z is selected from the group consisting of Arg, Ser, Thr, Asp, Glu, Tyr, Asn and Gln.

A82. The fusion protein of any of Paragraphs A71 to A81, wherein Z is Ser. A83. The fusion protein of any of Paragraphs A71 to A82, wherein Z is Arg.

A84. The fusion protein of any of Paragraphs A71 to A83, wherein the linker comprises the amino acid sequence Gly-X-Z and X is Gly.

A85. The fusion protein of any of Paragraphs A71 to A84, wherein P is Gly.

A86. The fusion protein of any of Paragraphs A71 to A85, wherein the linker comprises a diglycine sequence.

A87. The fusion protein of any of Paragraphs A71 to A86, which is expressed in a mammalian cell.

A88. A fusion protein comprising:
a) an amino acid sequence which, when the protein is administered, results in inhibition of adhesion of platelets to vitronectin; and
b) an antibody-derived amino acid sequence, wherein sequence (a) is linked at its C-terminus to the N-terminus of sequence (b) through a linker comprising a hydrophilic amino acid.

A89. The fusion protein of Paragraph A88, wherein the antibody-derived amino acid sequence is an Fc domain of an immunoglobulin.

A90. The fusion protein of Paragraph A88 or Paragraph A89, wherein the amino acid sequence which results in the inhibition of adhesion of platelets to vitronectin is an extracellular domain of GPVI.

A91. The fusion protein of any of Paragraphs A88 to A90, wherein the hydrophilic amino acid is selected from Arg, Ser, Thr, Lys, His, Glu, Asp and Asn.

A92. The fusion protein of any of Paragraphs A88 to A91, wherein the hydrophilic amino acid is Arg.

A93. A fusion protein comprising:
a) an amino acid sequence comprising an extracellular domain of GPVI or a variant thereof that is functional for binding to vitronectin;
b) a linker comprising an amino acid sequence Gly-Gly-Z, wherein Z is a hydrophilic amino acid; and
c) an amino acid sequence comprising an Fc domain of an immunoglobulin or a functional conservative part thereof.

A94. The fusion protein of Paragraph A93, wherein the amino acid sequence (a) is encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8); or
(iii) a nucleic acid sequence which differs from bases 1 to 807 of SEQ ID No. 2 (FIG. 8) by virtue of the degeneracy of the genetic code.

A95. The fusion, protein of Paragraph A93 or Paragraph A94, wherein amino acid sequence (b) is encoded by:
(i) a nucleic acid sequence of bases 808 to 816 of SEQ ID. No 2 (FIG. 8);
(ii) a nucleic acid sequence which hybridises to bases 808 to 816 of SEQ ID No. 2 (FIG. 8);
(iii) a nucleic acid sequence which differs from bases 808 to 816 of SEQ ID No. 2 (FIG. 8) by virtue of the degeneracy of the genetic code

A96. The fusion protein of any of Paragraphs A93 to A95, wherein the hydrophilic amino acid is selected from Arg, His and Lys.

A97. The fusion protein of any of Paragraphs A93 to A96, wherein amino acid sequence (c) is encoded by:
(i) a nucleic acid sequence of bases 817 to 1515 of SEQ ID. No. 2 (FIG. 8);
(ii) a nucleic acid sequence which hybridises to bases 817 to 1515 of SEQ ID No. 2 (FIG. 8); or
(iii) a nucleic acid sequence which differs from bases 817 to 1515 of SEQ ID No. 2 (FIG. 8) by virtue of the degeneracy of the genetic code.

A98. A protein having the characteristics of a protein obtained by expressing in a mammalian cell under non-reducing conditions a DNA sequence comprising in a 5′ to 3′ direction:
(i) a DNA sequence comprising bases 1 to 807 of SEQ ID No. 2 (FIG. 8) or a variant thereof in which said sequence encoded by bases 1 to 807 of SEQ ID No. 2 (FIG. 8) is replaced by a variant having vitronectin binding activity;
(ii) a DNA sequence comprising bases 808 to 816 of SEQ ID. No. 2 (FIG. 8); and
(iii) a DNA sequence comprising bases 817 to 1515 of SEQ ID No. 2 (FIG. 8) or a variant thereof in which said sequence encoded by bases 817 to 1515 of SEQ ID No. 2 (FIG. 8) is replaced by a variant having the properties of an Fc region.

A99. A dimer of a polypeptide, the polypeptide comprising
a) an extracellular domain of GPVI or a variant thereof that is functional for binding to vitronectin; and
b) a linker comprising an amino acid sequence Gly-Gly-Z, wherein Z is a hydrophilic amino acid; and
c) an Fc domain of an immunoglobulin or a functional conservative part thereof.

A100. A method of treating or preventing thrombosis in a subject, comprising administering to the subject a therapeutically effective amount of a fusion protein comprising:
a) an amino acid sequence which, when the protein is administered, results in inhibition of vitronectin-induced platelet activation; and
b) a antibody-derived amino acid sequence, wherein sequence (a) is linked at its C-terminus to the N-terminus of sequence (b) through a linker comprising a hydrophilic amino acid.

A101. The method of Paragraph A100, wherein amino acid sequence (a) comprises an extracellular domain of GPVI or a variant thereof that is functional for binding to vitronectin; and the antibody-derived amino acid sequence comprises an Fc domain of an immunoglobulin or a functional conservative part thereof.

A102. The method of Paragraph A100 or Paragraph A101, wherein the fusion protein comprises sequentially in an N-terminus to C-terminus direction, a first amino acid sequence, a second amino acid sequence and a third amino acid sequence wherein said first amino acid sequence comprises:
A) i) an amino acid sequence encoded by a nucleic acid sequence of bases 1 to 807 of SEQ ID No. 2 (FIG. 8);
   ii) an amino acid sequence encoded by a nucleic acid sequence which hybridises to bases 1 to 807 of SEQ ID No. 2 (FIG. 8) and which codes for a polypeptide which binds to vitronectin; or
   iii) an amino acid sequence encoded by a nucleic acid sequence which differs from bases 1 to 807 of SEQ ID No. 2 (FIG. 8) by virtue of the degeneracy of the genetic code and which binds to vitronectin;
      and wherein the second amino acid comprises
B) i) an amino acid sequence encoded by a nucleic acid sequence of bases 808 to 816 of SEQ ID No. 2 (FIG. 8);
   ii) a 3-mer amino acid sequence containing a hydrophilic amino acid or
   iii) an amino acid sequence encoded by a nucleic acid sequence which differs from bases 808 to 816 of SEQ ID No. 2 (FIG. 8) by virtue of the degeneracy of the genetic code;
      wherein the third amino acid sequence comprises:
C) i) an amino acid sequence encoded by a nucleic acid sequence of bases 817 to 1515 of SEQ ID. No. 2 (FIG. 8);
   ii) an amino acid sequence encoded by a nucleic acid sequence which hybridises to bases 817 to 1515 of SEQ ID No. 2 (FIG. 8) and which codes for a polypeptide which is functional as an Fc domain of an immunoglobulin; or
   iii) an amino acid sequence encoded by a nucleic acid sequence which differs from bases 817 to 1515 of SEQ ID No. 2 (FIG. 8) by virtue of the degeneracy of the genetic code and which is functional as an Fc domain of an immunoglobulin.

A103. A dimer of a polypeptide, the polypeptide comprising:
a) a first polypeptide which is capable of inhibiting adhesion of platelets to vitronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid.

A104. A dimer of a polypeptide, the polypeptide comprising:
a) a first polypeptide which is capable of inhibiting adhesion of platelets to vitronectin;
b) a second, antibody-derived polypeptide; and
c) a linker comprising an amino acid sequence Gly-X-Z or Z-P-Q, wherein X is an amino acid, P and Q are each independently amino acids provided that at least one of P and Q is Gly, and Z is a hydrophilic amino acid,
   wherein the first polypeptide binds to vitronectin competitively with platelet-bound GPVI, the second polypeptide comprises a hinge region of an immunoglobulin and is functional to prolong the plasma half-life beyond that of a protein consisting of the first polypeptide and the linker and wherein the linker comprises the amino acid sequence Gly-X-Z, wherein Z is selected from the group consisting of Arg, Ser, Thr, Asp, Glu, Tyr, Asn and Gln.

A105. A dimer of a polypeptide, the polypeptide comprising:
a) a first polypeptide which is capable of inhibiting adhesion of platelets to vitronectin;
b) a second, antibody-derived polypeptide; and
c) a linker comprising an amino acid sequence Gly-X-Z, wherein X is Gly and Z is selected from the group consisting of Lys and Arg,
   wherein the first polypeptide binds to vitronectin competitively with platelet-bound GPVI and wherein the second polypeptide comprises a hinge region of an immunoglobulin and is functional to prolong the plasma half-life beyond that of a protein consisting of the first polypeptide and the linker.

Included herein are polypeptides which comprise some or all of a GPVI protein which is capable of binding to fibronectin and/or vitronectin. Such a polypeptide may competitively inhibit or prevent platelet-bound GPVI binding to fibronectin and/or vitronectin. Exemplary are soluble GPVI proteins, i.e. soluble proteins comprising a sequence derived from GPVI. Such a soluble GPVI protein may be a GPVI fusion protein. For example the fusion protein may comprise an extracellular domain of GPVI fused to a heterologous peptide sequence. The heterologous peptide sequence may be an Fc constant region. Included herein is the use of a soluble GPVI protein, for example a GPVI-Fc fusion protein, to inhibit fibronectin activity or function. Also included herein is the use of a soluble GPVI protein, for example a GPVI-Fc fusion protein, to inhibit vitronectin activity or function.

An agent described herein may be used in various methods of treating vascular disorders. Advantageously, the agents of the disclosure bind effectively to an atherosclerotic disorder, particularly a ruptured or fissured plaque, when delivered locally, thus avoiding the necessity of systemic administration at clinical levels. Thus, there is disclosed local administration, e.g. by a catheter, of an agent of the disclosure to a site, or suspected site, of an atherosclerotic disorder. In one embodiment, such an agent may be administered to a patient via a catheter to treat, for example, plaque disruption (e.g. rupture, fission) stenosis or restenosis or other thrombotic complications. In one embodiment, the agent is a fusion protein comprising an extracellular domain of GPVI fused to an immunoglobulin Fc portion via a linker. In one embodiment, the fusion protein is PR-15, a fusion protein comprising the sequence disclosed in FIG. 7.

The administration of an agent of the present invention via a catheter may provide an alternative method of treating spontaneous or other types of arterial lesions to systemic antiplatelet therapy.

Advantageously, the agents of the disclosure bind to the target molecule with a high degree of affinity. Binding affinity of an agent for example an antibody or fusion protein may be measured using for example BIACORE systems. The strength of the binding of an agent to a peptide sequence of GPVI may be analysed using chemiluminescence and quantified by measuring signal intensity.

In the all of the embodiments of the invention described herein, the amino acid sequence of the ligand, for example, GPVI, fibronectin and/or vitronectin, to which the agent binds may be modified by one or more changes in sequence which do not eliminate the underlying biological function and utility of the agents as described herein.

The disclosure includes agents which have the function of inhibiting GPVI interaction and/or association with fibronectin and/or vitronectin and/or collagen, including all variant forms which have such functionality, in particular clinically useful functionality, especially ability to inhibit GPVI binding with one, two or particularly all three of fibronectin, vitronectin and collagen. For example, proteins and other poly(amino acids) may be derivatised as by glycosylation, for example, to modify their properties. Other modifications included within the disclosure include without limitation attachment of natural or synthetic polymers (e.g. a polyethylene glycol or dextran) albumin affinity tags (see for example Bioorg Med Chem. Lett. 2002 Oct. 21; 12(20):2883-6). Also included are peptides containing one or more d-amino acids.

### Indwelling

When blood comes into contact with foreign matters or injured tissue platelets may become activated and/or adhere to the foreign matter or tissue. As discussed above, monocytes may be recruited to damaged vascular tissue and cause damage to it. When a device is implanted into the body, it is likely to be exposed to blood and the implantation process will injure neighbouring tissue. Hence, it is desirable for component materials for implantable devices, and other medical articles or instruments which may come into contact with blood, to comprise or be treated with, e.g. coated or impregnated with, a product to interfere with—and thus inhibit—binding and/or activation of platelets and/or monocytes. Exemplary of such implantable devices are intravascular devices, as exemplified by artificial hearts, artificial cardiac valves, artificial blood vessel, pacemakers, defibrillators, neurostimulators, stents, catheters, guidewires, cannulas, pump-oxygenators, blood vessel by-pass tubes, intraaortic balloon pumping, transfusion instruments, extracorporeal circulation circuits and their components and so forth.

As used herein, an indwelling device may be any suitable medical substrate that can be placed in a human or veterinary patient. The device may not be limited to permanently implanted devices and can include any device that comes into contact with blood, for example a surgeon's knife.

In accordance with embodiments, a coating or impregnant can be formed on or in an indwelling device or prosthesis or other medical instruments or articles. For coatings including one or more active agents, the agent will be released, e.g. at a desired rate and for a predetermined duration of time, at the site of implantation.

Generally speaking, a bioactive agent can be coupled to the surface of a medical device by surface modification, embedding or integration and released from within polymeric materials (matrix-type), or surrounded by and released through a carrier (reservoir-type). The polymeric materials in such applications should optimally act as a biologically inert barrier and not induce further inflammation within the body.

A coating composition can be used to coat a device or implant surface using any suitable means, e.g., by dipping, spraying and the like. The suitability of the coating composition for use on a particular material, and in turn, the suitability of the coated composition can be evaluated by those skilled in the art, given the present description.

The polymeric materials should therefore be non-inflammatory and, in the case of a stent, capable of being stretched without flaking or delaminating from the stent, and be able to deliver the drug at a sustained, controlled and predictable rate. Polymer systems that can meet these requirements are, for example, biodegradable polymers, such as polyglycolic-polylactic acid, polyethyleneoxidepolybutylene terephthalate, and polyorthoester or non-degradable polymers, such as Biogold polyamine-heparin, and methacryloylphosphorylcholine-laurylmethacrylate, for example.

Several patents are directed to devices utilizing biodegradable or bioresorbable polymers as drug containing and releasing coatings, including Tang et al, U.S. Pat. No. 4,916,193 and MacGregor, U.S. Pat. No. 4,994,071. Other patents are directed to the formation of a drug containing hydrogel on the surface of an implantable medical device, these include Amiden et al, U.S. Pat. No. 5,221,698 and Sahatjian, U.S. Pat. No. 5,304,121. Still other patents describe methods for preparing coated intravascular stents via application of polymer solutions containing dispersed therapeutic material to the stent surface followed by evaporation of the solvent. This method is described in Berg et al, U.S. Pat. No. 5,464,650.

Exemplary implantable devices include but are not limited to drug-delivering vascular stents; other vascular devices (e.g., grafts, catheters, valves, artificial hearts, heart assist devices), implantable defibrillators, blood oxygenator devices; surgical devices e.g. blades, tissue-related materials, membranes, cell culture devices, chromatographic support materials, biosensors, shunts for hydrocephalus, wound management devices, endoscopic devices, infection control devices, orthopedic devices, dental devices, urological devices, colostomy bag attachment devices, ophthalmic devices, glaucoma drain shunts, synthetic prostheses, intraocular lenses, respiratory, peripheral cardiovascular, spinal, neurological, dental, and ear/nose/throat devices (e.g., ear drainage tubes), renal devices, and dialysis articles (e.g., tubing, membranes, grafts). Other contemplated devices include self-expanding stents (e.g., made from nitinol), balloon-expanded stents (e.g., prepared from stainless steel), degradable coronary stents, non-degradable coronary stents, peripheral coronary stents, urinary catheters (e.g., surface-coated with antimicrobial agents), penile implants, sphincter devices, urethral devices, bladder devices, renal devices, vascular implants and grafts, intravenous catheters (e.g., treated with antithrombotic agents), small diameter grafts, artificial lung catheters, electrophysiology catheters, anastomosis devices, vertebral disks, bone pins, suture anchors, hemostatic barriers, clamps, surgical staples/sutures/screws/plates/clips, atrial septal defect closures, electro-stimulation leads for cardiac rhythm management (e.g., pacer leads), glucose sensors (long-term and short-term), blood pressure and stent graft catheters, blood oxygenator tubing, blood oxygenator membranes, blood bags, birth control devices, breast implants, benign prostatic hyperplasia and prostate cancer implants, bone repair/augmentation devices, breast implants, cartilage repair devices, orthopedic joint implants, orthopedic fracture repairs, tissue adhesives, tissue sealants, tissue scaffolds, cerebral spinal fluid (CSF) shunts, dental implants, dental fracture repair devices, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, central venous access catheters, hemodialysis devices, catheter cuffs, anastomotic closures, vascular access catheters, cardiac sensors, uterine bleeding patches, urological catheters/stents/implants, in vitro diagnostics, aneurysm exclusion devices, neuropatches, Vena cava filters, urinary dilators, endoscopic surgical tissue extractors, atherectomy catheters, clot extraction catheters, percutaneous transluminal angioplasty (PTA) catheters, percutaneous transluminal coronary angioplasty (PTCA) catheters, stylets (vascular and non-vascular), coronary guidewires, drug infusion catheters, esophageal stents, circulatory support systems, angiographic catheters, transition sheaths and dilators, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, parental feeding catheters, vascular prosthesis including endoprosthesis, stent-graft, and endovascular-stent combinations, small diameter grafts, abdominal aortic aneurysm grafts, wound dressings and wound management device, hemostatic barriers, mesh and hernia plugs, patches, including uterine bleeding patches, atrial septic defect (ASD) patches, patent foramen ovale (PFO) patches, ventricular septal defect (VSD) patches, and other generic cardiac patches; ASD, PFO, and VSD closures, percutaneous closure devices, mitral valve repair devices, left atrial appendage filters, valve annuloplasty devices, central venous access catheters, vascular access catheters, drug infusion catheters, blood pressure and stent graft catheters; anastomosis devices and anastomotic closures, aneurysm exclusion devices, biosensors including glucose sensors, birth control devices, breast implants, cardiac sensors, infection control devices, membranes, tissue scaffolds, tissue-related materials, shunts including cerebral spinal fluid (CSF) shunts, glaucoma drain shunts, dental devices and dental implants, ear devices such as ear drainage tubes, tympanostomy vent tubes, ophthalmic devices, cuffs and cuff portions of devices including drainage tube cuffs, implanted drug infusion tube cuffs, catheter cuff, sewing cuff; spinal and neurological devices, nerve regeneration conduits, neurological catheters; neuropatches, orthopedic devices such as orthopedic joint implants, bone repair/augmentation devices, cartilage repair devices, urological devices and urethral devices such as urological implants, bladder devices, renal devices and hemodialysis devices, colostomy bag attachment devices, biliary drainage products.

Other implantable devices may be categorised, such as belonging to the groups medical devices, dental devices, orthopedic devices, diagnostic devices, surgical devices, cell culture devices, urological devices, wound management devices, endoscopic devices, infection control devices, urological devices, colostomy bag attachment devices, ophthalmic devices, renal devices and dialysis.

The devices as listed above may be microporous.

A device may comprise nano bumps in order to closer mimic a smooth muscle vessel.

Indwelling devices e.g. implants, suitably further comprise a further bioactive agent or drug material, e.g. one selected from the group consisting of protein kinase inhibitors, antiproliferative agents, antimitotic agents, antibiotics, antimetabolites, anticoagulants, fibrolytic agents, antimigratory agents, antisecretory agents, anti-inflammatory agents, non-steroidal agents, angiogenic agents, anti-angiogenic agents, immunosuppressive agents, pyrimidine analogues, purine analogues, spleen tyrosine kinase (SYK) inhibitors and combinations thereof.

An aspect of the invention is a method of inhibiting or preventing restenosis, thrombosis, atherogenesis, atheroprogression, atherosclerosis, and/or vascular inflammation in a patient, said method comprising implanting in said patient an intravascular device comprising a direct or indirect GPVI inhibitor adapted to be exposed and/or released when the device is implanted.

It will be appreciated from the aforegoing that the products of the disclosure are proposed for use in the inhibition of blood-foreign surface interactions.

One particular device is a stent.

### Stent

Intravascular stenting of arteries is one of the most frequent operations in cardiovascular surgery. Stents (metal woven fabric cylinders) are inserted into a vessel with injured inner wall to avoid its destruction, thrombus formation or vasoconstriction. Unfortunately, metal stents provoke thrombus formation. To enhance stent biocompatibility, it is possible either to modify metal surface of implant or to apply biocompatible coatings.

The coronary artery response to stent implantation leads to a complex and largely predictable sequence of events. The events are related to inflammation and repair processes, which are known to be natural compensatory mechanisms. The temporal sequence of events to stent implantation can be divided into acute and chronic phases. In the acute phase, thrombosis disturbances predominate. In the chronic phase, tissue remodeling reaches primacy. Thus, while thrombosis takes place early and at the vessel lumen boundary during the acute phase, neointimal hyperplasia and restenosis are protracted in duration, and deeper into the vessel wall during the chronic phase.

However, a stent having a coating or impregnant which contains at least one disclosed agent is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

Recently, the use of drug-eluting stents (DES) in percutaneous coronary interventions has received much attention. DES are medical devices that present or release bioactive agent into their surroundings (for example, luminal walls of coronary arteries).

In addition to anticoagulants, incorporation of antimicrobial agents (e.g., antibiotics such as vancomycin or norfloxacin) into a surface coating, may also prove beneficial as additional active agents.

In one aspect, the present agent is for the treatment of a thromboembolic disorder which may be selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophiebitis, arterial embolism, coronary arterial thrombosis, cerebral arterial thrombosis, cerebral embolism, kidney embolism, pulmonary embolism, and thrombosis resulting from (a) prosthetic valves or other implants, (b) Indwelling catheters, (c) stents, (d) cardiopulmonary bypass, (e) hemodialysis, or (f) other procedures in which blood is exposed to an artificial surface that promotes thrombosis

### Dual Action

In another aspect, the product of the present invention has a dual action, for example, it has a direct or indirect diagnostic function or a therapeutic (Including prophylactic) function by another mode of action.

Accordingly, the disclosure includes an agent which comprises:
a first portion capable of binding to an intravascular GPVI-binding domain; and
a second portion which is capable of binding to a site other than a GPVI-binding domain and/or comprises a therapeutic or diagnostic moiety,
   or a combination thereof.

The second portion may be capable of binding to a binding site for a platelet-bound ligand other than GPVI, for example GPIa, GPIb, GPIc, GPIIa, GPIIb, GPIIIa, GPIX, GPIIb/IIIa, GPIa/IIa, GPIV, GPIc/IIa, GPIb/IX. The second portion may comprise an amino acid sequence derived from a platelet receptor other than GPVI, or a sequence having homology to at least part of such sequence.

To be mentioned are agents having a second portion which:
is functional to interfere with, modulate or confer the activity of platelet derived growth factor (PDGF), glycoprotein IBα, thrombomodulin, vascular epidermal growth factor, transforming growth factor-β1, basic fibroblast growth factor, angiotensin II, factor VIII, von Willebrand factor; or
comprises a protein kinase inhibitor, an antiproliferative agent, an antimitotic agent, an antibiotic, an antimetabolite, an anticoagulant, a fibrolytic agent, an antimigratory agent, an antisecretory agent, an anti-inflammatory agent, a non-steroidal agent, an angiogenic agent, an anti-angiogenic agent, an immunosuppressive agent, a pyrimidine analogue, a purine analogue, or a spleen tyrosine kinase (SYK) inhibitor.

The second portion may confer anti-platelet activity, for example inhibitory activity against platelet adhesion and/or platelet aggregation. The second portion may confer anticoagulant activity.

In embodiments, the second portion comprises a matrix metalloprotease inhibitor.

Included are embodiments in which the second portion is either proliferative (particularly to aid wound healing) or anti-proliferative (to help prevent atherosclerosis or (re)stenosis.

The disclosure includes embodiments in which the second portion comprises an amino acid sequence derived from platelet derived growth factor (PDGF), a platelet receptor protein (e.g. a platelet-bound glycoprotein) hirudin, thrombomodulin, vascular epidermal growth factor, transforming growth factor-1, basic fibroblast growth factor, angiotensin II, factor VIII, von Willebrand factor, tick anticoagulant protein (TAP) or nematode anticoagulant protein (NAP), or a sequence having homology to at least part of such sequence.

The second portion may comprise a domain selected from one or more of the group consisting of protein kinase inhibitors, antiproliferative agents, antimitotic agents, antibiotics, antimetabolites, pyrimidine analogs, purine analogs, anticoagulants, fibrinolytic agents, antiplatelet agents, antimigratory agents, antisecretory agents, anti-inflammatory agents, non-steroidal agents, immunosuppressive agents, angiogenic agents, ACE inhibitors, actin inhibitors, analgesics, anesthetics, anti-hypertensives, anti polymerases, antisecretory agents, anti-AIDS substances, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidants, anti-parasite and/or anti-Parkinson substances, antiproliferatives (Including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, opioids, photodynamic therapy agents, prostaglandins, remodeling inhibitors, statins, steroids, thrombolytic agents, tranquilizers, vasodilators, and vasospasm inhibitors, and combinations thereof.

The dual action products may include an additional agent which interacts with other factors, including protein factors, that are involved in the clotting cascade which may include coagulation factors I-XIII (for example, fibrinogen, prothrombin, tissue thromboplastin, calcium, proaccelerin (accelerator globulin), proconvertin (serum prothrombin conversion accelerator), antihemophilic factor, plasma thromboplastin component, Stuart factor (autoprothrombin C), plasma thromboplastin antecedent (PTA), Hageman factor, and fibrin-stabilizing factor (FSF, fibrinase, protransglutaminase)).

The second portion may comprise an immunosuppressive agent, e.g. rapamycin or a rapamycin derivative. Rapamycin may be used for the treatment of AV graft stenosis since it has been shown to significantly reduce in-stent restenosis and prevent chronic organ rejection. Rapamycin is a potent inhibitor of cytokine and growth factor-mediated smooth muscle cell proliferation.

Other surface adhesion molecule or cell-cell adhesion molecules may also function to promote coagulation or thrombosis. Exemplary cell adhesion molecules or attachment proteins (such as extracellular matrix proteins) include laminin, collagen, elastin, tenascin, fibrinogen, thrombospondin, osteopontin, von Willebrand Factor, bone sialoprotein (and active domains thereof), or a hydrophilic polymer such as hyaluronic acid, chitosan or methyl cellulose, and other proteins, carbohydrates, and fatty acids. Exemplary cell-cell adhesion molecules include N-cadherin and P-cadherin and active domains thereof. It is therefore contemplated that certain products of the present invention will interact with at least one of these.

In a further aspect of the present disclosure there is provided a conjugate comprising an agent of the present disclosure linked to one or more heterologous molecules. A conjugate of the present disclosure may be used as, for example but not limited to, a drug delivery agent or a multifunctional therapeutic agent. In a further aspect of the present disclosure, there is provided a pharmaceutical composition comprising a conjugate according to the present disclosure. Methods of treatment and uses of the conjugates to manufacture a medicament to treat a disorder are also included in the present disclosure. Further aspects of the disclosure include kits comprising conjugates of the present disclosure, and pharmaceutical compositions comprising a conjugate of the present disclosure together with another active agent or drug. Such active agents and drugs include, but are not limited to, those described herein.

In one embodiment, the conjugate comprises (1) an agent, which is a polypeptide, linked to (2) a second polypeptide. In a particular embodiment, the second polypeptide has a different activity from the polypeptide agent. In one embodiment, the agent is a soluble GPVI polypeptide, and is optionally a GPVI fusion protein. In an embodiment, the conjugate comprises a GPVI-Fc fusion protein linked to a second polypeptide. In an alternative embodiment, the agent is linked to a non-polypeptide molecule such as a small molecule drug. The conjugate, in other embodiments, comprises other agents of the disclosure as described herein.

Thus, conjugates of the disclosure may be bi- or multifunctional in their activities. For example, in one embodiment, the agent is conjugated to a molecule which is capable of modulating the expression or activity of factors involved in, for example but not limited to, blood coagulation, leukocyte recruitment, immune system activation, tissue fibrosis and tumorigenesis.

In this way, the conjugates of the disclosure may be rendered multifunctional so that they are active against more than platelet interaction with the vasculature, for example another component in the clotting cascade (eg thrombin activity), or the intracellular signaling cascade (eg growth factor).

In one embodiment, the conjugate comprises the agent linked to a cytotoxic drug or toxin. In one embodiment, the agent is a monoclonal antibody which is capable of being internalized by a target cell. Upon administration to a patient, the conjugate binds to target cells through their antibody portions and becomes internalized, allowing the drugs or toxins to exert their cytotoxic or cytostatic effects.

In this disclosure, the components of the conjugate may be covalently bonded to each other either directly or through a linking group. Thus, the conjugate may comprise a linker between the agent and the heterologous molecule. Alternatively, the agent and heterologous molecule may be linked directed. In one embodiment, the linker is selected from the group consisting of: a flexible linker, an inflexible linker, a linker which is capable of being cleaved further. It will be apparent to one skilled in the art that alternative linkers can be used to link first and second parts of the conjugate. A flexible linker may be about 20 or fewer amino acids in length.

In a further embodiment of the present disclosure, the conjugate comprises an agent which is capable of acting as a presenter protein ligand. In the subject methods, a conjugate which is a bifunctional molecule is synthesized by covalently linking an endogenous presenter protein ligand to a drug moiety, either directly or through a linking group. The host endogenous presenter protein ligand of the conjugate may then act, when administered, to direct the drug moiety to a target presenter protein in the host. Thus, for example, in one embodiment the conjugate comprises an agent which binds to a protein selected from GPVI, fibronectin, vitronectin and/or collagen, linked to a drug moiety. When administered, the agent may then act to direct the drug moiety to presenter protein, namely a protein which interacts with the agent. Thus, the agent binds to a protein which is a target for the drug moiety. For example, in one embodiment, the agent binds to one or more of the proteins selected from GPVI, fibronectin, vitronectin and collagen. In this embodiment, the drug moiety is active against one or more of the proteins to which the agent binds. Upon administration, the conjugate binds to the presenter protein to produce a binary complex. The presenter protein to which the ligand of the conjugate binds may be any protein that is present in the host at the time the conjugate is introduced to the host, i.e. the presenter protein will be endogenous to the host.

In one embodiment, the conjugate comprises (1) a GPVI fusion protein, linked to (2) a drug moiety or a peptide moiety. In a particular embodiment, the conjugate comprises a GPVI fusion protein which comprises an extracellular domain of GPVI linked to an Fc portion via a linker comprising a Gly-Gly-Arg sequence. In alternative embodiments, the conjugate comprises a GPVI fusion protein as described herein.

It is disclosed herein that soluble GPVI is capable of adhering to the location where it is locally released. Thus, it is envisaged that use of a soluble GPVI, for example in the form of a GPVI fusion protein, in a conjugate with a drug moiety or peptide moiety would act to ensure that the drug or peptide was not swept away in the blood following local delivery. Thus, the use of soluble GPVI in a conjugate with a drug moiety or peptide moiety may act to address the problem of locally delivered drugs being swept away by the blood after delivery.

The term “soluble GPVI” is considered to include any protein or polypeptide which comprises at least an extracellular domain of GPVI or functional portion thereof. Soluble GPVI may include, but is not limited to, GPVI fusion proteins.

The drug moiety may include, but is not limited to, any of the compounds or drug described herein. In one embodiment, the conjugate comprises a cleavable linker between the agent and the drug moiety or peptide moiety. In this embodiment, the agent may act to “slow down” or retard the removal of the drug by the blood stream from the local area of delivery.

The drug moiety may be any molecule, as well as a fragment for example a binding portion, thereof, that is capable of modulating a biological process in a living host, either by itself or in the context of the presenter protein/conjugate binary complex. Generally, the drug moiety is a small organic molecule that is capable of binding to a target of interest.

The drug moiety of the conjugate will include one or more functional groups necessary for structural interaction with the target, e.g. groups necessary for hydrophobic, hydrophilic, electrostatic or even covalent interactions, depending on the particular drug and its intended target. Where the target is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The drug moiety will also comprise a region that may be modified and/or participate in covalent linkage to the other components of the conjugate, such as the presenter protein ligand or linker, without substantially adversely affecting the moiety's ability to bind to its target.

The drug moiety of the conjugate may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

There is provided herein a diagnostic or visualisation agent comprising an agent of the present disclosure linked to a moiety to enable the location of the agent to be detected e.g. a detectable moiety or a moiety for binding to a dectable moiety or entity.

The term “detectable moiety” may include molecules that are readily detectable by a medical imaging system. In one embodiment, the detectable moiety is selected from the group consisting of electron-opaque molecules, for example gold particles, radioisotopes, for example technetium, and stable isotope-labeled molecules. The detectable moiety can be any one which is capable of producing, either directly or indirectly, a detectable signal.

In an embodiment, the detectable moiety is selected from a radiolabel, a fluorophore, a chromophore, an imaging agent and a metal ion. For example, the detectable moiety may be a radioisotope, for example 1-123, 1-124 and 1-131, Tc-99m, Re-186 and Re-188, particularly Tc-99m; a fluorescent or chemiluminescent compound, such as fluorescein isothiocyanate, rhodamine, or luciferin; or an enzyme, such as alkaline phosphatase, beta-galactosidase or horseradish peroxidase. In embodiments, there is excluded radioiodine (radioactive iodine isotopes) as a component of an agent of the disclosure. Particularly, radioiodine is excluded when the agent is the agent of Paragraph 15 below.

Radio-labelling of biomolecules has been used as a means to track and detect the pathway or location of a particular biomolecule when administered to a patient or subject. Such radio-labelled biomolecules are capable of emitting low levels of radiation, which can be detected and pin-pointed to a target organ or other substrate.

Radionuclides such as rhenium-186m and, particularly, technetium-99m, are useful for biomolecule labelling since they are known to form relatively stable bonds with a variety of biomolecules and may be used in the imaging of patients.

Any method known in the art for conjugating the agent to the detectable moiety may be employed, including those methods described by Hunter, et al, Nature 144:945 (1962); David, et al, Biochemistry 13:1014 (1974); Pain, et al, J. Immunol. Meth. 40:219 (1981); and Nygren, J. Histochem and Cytochem. 30:407 (1982). The detetable moiety may be administered as a separate entity which binds to the visualisation imaging or locating agent in vivo through a binding partner e.g. an antibody product.

In one embodiment, the agent is a protein, antibody or fragment thereof. In one embodiment, the protein is a soluble GPVI protein, for example a GPVI fusion protein. Other agents described herein are also encompassed in this aspect of the disclosure.

A method for diagnosis is also provided in which the imaging agent is administered to a subject suspected of having an atherosclerotic disorder, for example a dysfunctional endothelium, microscopic atherosclerotic lesions and/or macroscopic atherosclerotic lesions, and the distribution of the imaging agent within the body of the subject is measured or monitored.

The imaging agents of the disclosure are useful for in vivo imaging, wherein an imaging agent linked to a detectable moiety such as a radio-opaque agent or radioisotope is administered to a subject, preferably into the bloodstream, and the presence and location of the imaging agent in the host is assayed. This imaging technique is useful in the staging and treatment of atherosclerosis or thrombosis or other cardiovascular disorders. The technique may also be useful in the staging and treatment of cancers. The imaging agent may comprise any moiety that is detectable in a host, whether by nuclear magnetic resonance, radiology, or other detection means known in the art.

The imaging agent may further comprise polymerized vesicles, which are composed of polymerized lipids and lipids that are chelated to gadolinium. These vesicles counteract the instability of pure liposomes while avoiding the clumping and tangling of pure polymers. The resulting vesicle is round and flexible, and it interacts well with surfaces where biomarkers such as collagen, fibronectin and/or vitronectin may be located.

Agents, for example proteins and antibodies, specific to the biomarker can be attached to the vesicle and injected into the blood. Using polymerized vesicles, the amount of imaging agent accumulating at target sites may increase substantially over imaging agents without the vesicles.

The present disclosure further includes methods of amplifying the signal produced by the imaging agent. These are important to ensure that a signal sufficient for detection is produced. Thus, accumulation of the imaging agent at the target site may be helpful. In this instance, use in the imaging agent of an agent which binds to plural binding sites as described herein, e.g. selected from fibronectin, vitronectin and collagen, will potentially ensure that the imaging agent accumulates at site which expresses fibronectin, vitronectin and/or collagen in quantities sufficient to produce a detectable signal. Alternatively or in addition, the imaging agent may comprise a molecule which causes amplification of the imaging agent, for example an enzyme.

The present disclosure also includes methods of detecting an imaging agent of the present disclosure comprising administering the imaging agent to a subject and using a method to detect the signal produced by the imaging agent. Such methods may include CT, MR, optical imagining, ultrasound, and PET.

Thus, in one aspect of the present disclosure there is provided a method of diagnosing one of the following conditions in a subject:

(a) inflamed endothelium of a blood vessel;
(b) microscopic atherosclerotic lesions;
(c) macroscopic atherosclerotic lesions; and
(d) other disorders disclosed herein,
comprising administering an imaging agent of the present disclosure to a subject and detecting a signal produced by the imaging agent.

In a further aspect of the disclosure there is provided a method for preparing a hybridoma cell-line producing monoclonal antibodies according to the disclosure comprising the steps of:
i) immunising an immunocompetent mammal with an immunogen for example, GPVI, fibronectin, vitronectin or fragments thereof;
ii) fusing lymphocytes of the immunised immunocompetent mammal with myeloma cells to form hybridoma cells;
iii) screening monoclonal antibodies produced by the hybridoma cells of step (i) for binding activity to the amino acid sequences of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The ability of an agent to bind human glycoprotein VI, fibronectin and/or vitronectin or fragments thereof, may be tested by immunoassay. Any form of direct binding assay is suitable. In one such assay, the human glycoprotein VI, fibronectin and/or vitronectin or alternatively the agent is labeled. Suitable labels include radioisotopes such as ¹²⁵I, enzymes such as peroxidase, fluorescent labels such as fluorescein, and chemiluminescent labels. Typically, the other binding partner is insolubilized (for example, by coating onto a microtiter plate) to facilitate washing. After combining the labeled component with the insolubilized component, the solid phase is washed and the amount of bound label is determined. To conduct the inhibition assays, the agent is titered for its ability to decrease the binding of, for example, an anti-GPVI antibody e.g. hGP 5C4 Fab to human glycoprotein VI, or human glycoprotein VI to fibronectin and/or vitronectin. Either of the binding pairs in the reaction to be inhibited is labeled, while the other is typically insolubilized in order to facilitate washing.

### Use of the Products of the Disclosure

### Disease Treatment

The present invention relates in some respects to treating a subject with a condition in which inhibition cardiovascular disease, particularly atherosclerosis and/or thrombosis, is required or desired.

As previously described, the referenced agents find application in the primary prophylaxis of atherosclerotic disorders. They find application in the secondary prophylaxis of atherosclerotic disorders, after an atherosclerotic event or suspected atherosclerotic event, e.g. atherothrombosis. The disclosures elsewhere in this specification of those and other medical uses, including uses associated with surgery, will not be repeated here.

Particular disease states which may be mentioned include the therapeutic and/or prophylactic treatment of venous thrombosis (e.g. DVT) and pulmonary embolism, arterial thrombosis (e.g. In myocardial infarction, unstable angina, thrombosis-based stroke and peripheral arterial thrombosis) and systemic embolism usually from the atrium during arterial fibrillation or from the left ventricle after transmural myocardial infarction, or caused by congestive heart failure; prophylaxis of re-occlusion (le thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular thrombosis caused by bacteria, multiple trauma, or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in hemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosderosis such as coronary arterial disease, cerebral arterial disease, peripheral arterial disease, reperfusion damage, and restenosis after percutaneous trans-luminal angioplasty (PTA).

Conditions which may be treated include thrombosis, especially DVT, including distal and proximal DVT. The present disclosure finds particular utility in the prophylactic treatment of DVT resulting from surgery, such as gastrointestinal, or orthopaedic, surgery (e.g. hip or knee replacement). This includes DVT resulting from immobilisation after surgery.

Suitable doses of the described agents, in the therapeutic and/or prophylactic treatment of mammalian, especially human, patients may be determined routinely by the medical practitioner or other skilled person. In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this disclosure.

There is further provided a package or kit of parts comprising:

(1) an agent described herein; together with
(2) instructions to use the agent in a method described herein.

The package defined herein may comprise more than one dosage unit, in order to provide for repeat dosing. If more than one dosage unit is present, such units may be the same, or may be different in terms of the dose of active agent composition and/or physical form.

A further aspect of the disclosure provides a method of treatment of a condition in which inhibition of thrombin is required or desired, which comprises administration of:

(a) a pharmaceutical formulation including a low molecular weight thrombin inhibitor, or a pharmaceutically acceptable derivative thereof, in admixture with a pharmaceutically acceptable adjuvant, diluent or carrier; in conjunction with
(b) an agent of the disclosure,
to a patient suffering from, or susceptible to, such a condition.

For the avoidance of doubt, as used herein, the term “treatment” includes therapeutic and/or prophylactic treatment.

In other aspects the disclosure provides an agent or a ligand as hereinbefore described for use as a pharmaceutical.

For most applications, polypeptides of the disclosure are at least partially purified from other cellular constituents. Preferably, the polypeptide is at least about 50% pure. as a weight percent of total protein. More preferably, the protein is at least about 50-75% pure. For clinical use, the polypeptide is preferably at least about 90% pure, typically 98% pure or more.

In further aspects, there is provided a pharmaceutical formulation comprising an agent, a ligand or a humanised antibody as hereinbefore described. The formulation may contain at least one additional pharmaceutically acceptable component, e.g. an excipient, diluent or carrier. The formulation may be for parenteral administration. Alternatively, the formulation is for oral administration. In this embodiment, the agent may be, for example, a single-chain antibody or other antibody fragment which is suitable for oral administration; it may by way of example be a VHH or aAb.

In one embodiment, the product which comprises an agent of the disclosure, may be comprised within a pharmaceutical formulation which is for chronic administration. The pharmaceutical formulation may be for the treatment of atherosclerosis in a subject who has atherosclerotic plaques or who does not have atherosclerotic plaques but is at risk thereof.

Further included is a pharmaceutical formulation comprising a ligand described herein, for example, GPVI, fibronectin and/or vitronectin or fragments thereof; in embodiments the formulation is a composition comprising the ligand and a pharmaceutically acceptable diluent, carrier or excipient. In another aspect there are provided the described ligands for use as a pharmaceutical. The formulation may be an intravenous formulation.

The agents of the present disclosure may also be used in methods of inhibiting platelet aggregation, wherein the method comprises contacting platelets with an effective amount of the agent as described herein. The method may involve platelets that are in vitro or in vivo.

The agents of the present disclosure may also be used in methods of treating a disease or disorder associated with pathological, dysfunctional or non-pathological interaction between platelet-bound GPVI and fibronectin. The method may comprise administering an agent, or a product which comprises an agent, which inhibits the binding of platelet bound GPVI to fibronectin to a subject with the disease or disorder or at risk of developing the disease or disorder. Preferably, a therapeutically effective amount of the agent or product is administered.

The agents of the present disclosure may also be used in methods of treating a disease or disorder associated with pathological, dysfunctional or non-pathological between platelet-bound GPVI and vitronectin comprising administering an agent or a product which comprises an agent which inhibits the binding of platelet bound GPVI to vitronectin to a subject with the disease or disorder or at risk of developing the disease or disorder. Suitably, a therapeutically effective amount of the agent or product is administered.

In one embodiment, the treatment is therapeutic or prophylactic and the disease or disorder is selected from acute coronary syndromes, cardiovascular thrombosis, cerebrovascular thrombosis, unstable angina, stable angina, angina pectoris, embolus formation, deep vein thrombosis, hemolytic uremic syndrome, hemolytic anemia, acute renal failure, thrombolytic complications, thrombotic thrombocytopenic purpura, disseminated intravascular comgelopathy, thrombosis, coronary heart disease, thromboembolic complications, myocardial infarction, restenosis, and atrial thrombosis formation in atrial fibrillation, chronic unstable angina, transient ischemic attacks and strokes, peripheral vascular disease, arterial thrombosis, pre-eclampsia, embolism, restenosis and/or thrombosis following angioplasty, carotid endarterectomy, anastomosis of vascular grafts, and chronic exposure to cardiovascular devices.

In one embodiment, the treatment is for therapy or prevention of thromboembolism and reclusion occurring during and after thrombolytic therapy, after angioplasty, and after coronary artery bypass.

The agents of the present disclosure may also be used in methods of preventing or retarding initiation and/or progression of atherosclerotic lesions in a subject with the atherosclerotic lesions or at risk of developing atherosclerotic lesions. Preferably, a therapeutically effective amount of the agent or a product comprising the agent is administered.

The agents of the present disclosure may also be used in methods of treating or reducing advanced atherosclerotic lesions in a subject. Preferably, a therapeutically effective amount of the agent or a product comprising the agent is administered.

The agents of the present disclosure may also be used in the manufacture of a medicament for the treatment of a disease or disorder associated with pathological, dysfunctional or non-pathological interaction between platelet-bound GPVI and vitronectin.

The agents of the present disclosure may also be used in the manufacture of a medicament for the treatment of a disease or disorder associated with pathological, dysfunctional or non-pathological interaction between platelet-bound GPVI and fibronectin.

In one embodiment, the treatment is therapeutic or prophylactic and the a disease or disorder is selected from acute coronary syndromes, cardiovascular thrombosis, cerebrovascular thrombosis, unstable angina, stable angina, angina pectoris, embolus formation, deep vein thrombosis, hemolytic uremic syndrome, hemolytic anemia, acute renal failure, thrombolytic complications, thrombotic thrombocytopenic purpura, disseminated intravascular comgelopathy, thrombosis, coronary heart disease, thromboembolic complications, myocardial infarction, restenosis, and atrial thrombosis formation in atrial fibrillation, chronic unstable angina, transient ischemic attacks and strokes, peripheral vascular disease, arterial thrombosis, pre-eclampsia, embolism, restenosis and/or thrombosis following angioplasty, carotid endarterectomy, anastomosis of vascular grafts, and chronic exposure to cardiovascular devices.

In one embodiment, the medicament is for treating or preventing thromboembolism and reocculsion occurring during and after thrombolytic therapy, after angioplasty, and after coronary artery bypass.

The methods of the present invention have particular usefulness in in vivo applications. For example, agents which bind GPVI with fibronectin and/or vitronectin and/or collagen can be used in the treatment of any disease, state or condition involving the interaction of GPVI with fibronectin and/or vitronectin and/or collagen.

It is considered that agents of the disclosure which interrupt the interaction between GPVI and fibronectin and/or vitronectin may be useful in treating or preventing atherosclerosis. Based on the recent improvements in imaging techniques by intravascular ultrasound or nuclear magnetic resonance imaging, it is possible to identify patients with atherosclerosis being at risk of acute clinical complications such as acute coronary or carotid syndrome, whereby the patients have active lesions as possible causes for intravascular thrombosis. It is then possible by the present teachings to prevent or delay the formation of intravascular thrombosis by the administration of a medicament containing the agents of the invention, advantageously without undesired or unacceptable side effects.

The occurrence of atherosclerotic lesions can be investigated e.g. by intravascular ultrasound or thermography (e.g., Fayed and Fuster, Clinical imaging of the high-risk or vulnerable atherosclerotic plaque. Circulation 2001; 89:305-316) or nuclear resonance imaging (Helft et al., Progression and Regression of Atherosclerotic Lesions. Circulation 2002; 105:993-998). Such lesions are highly probable in patients with acute coronary or carotid syndromes, and the risk of the reoccurrence of acute clinical complications such as myocardial infarction or stroke is very high, decreasing progressively with increasing time distance from the primary event.

Accordingly, further aspects of the disclosure provide methods of treatment comprising, administration of an agent as provided, pharmaceutical compositions comprising such a agent, and use of such an agent in the manufacture of a medicament for administration, for example in a method of making a medicament or pharmaceutical composition comprising formulating the agent with a pharmaceutically acceptable excipient.

The agents of the disclosure, particularly those which interact with, particularly bind with, one or both of fibronectin and vitronectin, may be used in the prophylaxis of atherosclerotic disorders, e.g. initiation or development of atherosclerosis, in patients having one or more markers of vascular inflammation. Patients identified as having such markers, particularly at a clinically significant level, may be at risk of an atherosclerotic event, e.g. angina or myocardial infarction.

Since inflammation is associated with vascular disease, the measurement of inflammatory markers may be a powerful method for identifying increased inflammatory activity and predicting future cardiovascular events in individuals. Such markers include liver proteins, such as high-sensitivity-C-reactive protein (hs-CRP), serum amyloid A (SAA), secretory phospholipase A2 and fibrinogen; cytokines, such as interleukin-1 (IL-1), interleukin-6 (IL-6) and tumor necrosis factor-alpha (TNF-α); and vascular markers, such as soluble intercellular adhesion molecule-1 (sICAM-1). Indeed, to date, these variables have been identified as prospective risk markers of cardiovascular disease, and together provide a consistent message of the involvement of inflammation in cardiovascular risk. Other identified markers include brain natriuretic peptide (BNP) and N-terminal pro-BNP (NT-proBNP) and elevated ADMA is now widely recognized as a risk marker for vascular disease. Other markers of vascular disease may also be used to predict future vascular disorders in individuals.

Clinical indications in which an agent of the present disclosure may be used to provide therapeutic benefit include any condition in which fibronectin and/or vitronectin and/or collagen recognition by GPVI potentially has pathological consequences, for example in cardiovascular conditions such as thrombosis, including for example arterial thrombosis occurring at a blood vessel wall disease (e.g. coronary artery thrombosis, which causes myocardial infarction). Similar thrombotic processes may occur in other serious conditions at diverse anatomical locations, for instance in the cerebral vasculature, leading to stroke, or in the peripheral extremities. In the latter case for instance, patients with intermittent claudication may be treated. Agent-mediated blockade of GPVI may be used and be beneficial during therapeutic procedures which induce damage to the blood vessel wall, for instance vascular surgery. Examples of vascular surgery may include, but are not limited to, coronary artery bypass grafting, balloon angioplasty and stenting. In other, unrelated, disease processes, circulating platelets may be exposed to fibronectin or vitronectin where they may contribute to local thrombotic effects and to the inflammatory processes which ensue. An example of the latter occurs in hepatitis where the hepatic circulation is compromised by the disease. In addition diseases of generalised platelet activation such as thrombocytopenic purpura and haemolytic uraemic syndrome and other clinical conditions with disseminated intravascular coagulation may be ameliorated. Furthermore multi-organ damage because of arterial insufficiency in patients with homozygous sickle disease may be beneficially affected by inhibiting the activation of platelets via GPVI. Similarly kidney damage by platelet and fibrin disposition on the glomerular membrane and other conditions such as micro-angiopathic vasculitides may be treated by agent-mediated GPVI and/or fibronectin and/or vitronectin blockade.

Venous thromboembolic events (VTE) are often associated with cancer and contribute to the increased mortality in these patients. It is considered that methods, products, agents and uses of the present disclosure may be used to treat or alleviate or prevent VTE in patients suffering from cancer.

In fact, venous thromboembolism, which includes deep vein thrombosis (DVT) and resulting pulmonary emboli (PE) is the second leading cause of death in people affected by cancer, after death from the cancer itself. Thrombosis is a common complication in patients with malignant disease, with up to 15 percent of patients developing a clot with major symptoms and serious outcomes. The occurrence of a blood clot can have a significant impact on a cancer patient's quality of life and in some instances can be life-threatening. Up to 1 in 7 cancer patients will develop thromboembolism at some point. Types of treatment may include;
Thromboprophylaxis (prevention of thrombosis) in conjunction with surgery
Treatment of acute deep vein thrombosis
Unstable coronary artery disease (UCAD), i.e., unstable angina and non-Q-wave myocardial infarction
Prevention of clotting in the extracorporeal system during hemodialysis and hemofiltration in connection with acute renal failure or chronic renal insufficiency
Extended treatment of symptomatic VTE to prevent recurrence of venous thromboembolism in patients with cancer.

Cancer patients frequently develop alterations in hemostasis including platelet abnormalities, abnormal activation of the coagulation cascade, release of plasminogen activator, and decreased hepatic synthesis of anticoagulant and coagulant proteins. Platelet aggregation and activation in cancer patients may be triggered by the shedding of membrane glycolipids into the host circulation by tumor cells. Platelet activation and aggregation can then lead to thrombus formation in the cancer patient.

Thrombotic manifestations in cancer patients may present as one of the following: migratory thrombophlebitis or Trousseau syndrome, deep venous thrombosis (DVT), pulmonary embolism, TTP/HUS, arterial thrombosis, and DIC.

As previously mentioned, the agents of the disclosure have application in primary prophylaxis of thrombosis, and this includes such prophylaxis in cancer patients.

### Migratory Thrombophiebitis (Trousseau Syndrome)

Trousseau syndrome is a classically described variant form of venous thrombosis characterized by a recurrent and migratory pattern preferentially involving superficial veins of the arms and chest. Trousseau syndrome is highly associated with adenocarcinomas and laboratory evidence of DIC. Migratory thrombophlebitis has also been associated with the use of somatostatin or ocreotide therapy for malignant carcinoid syndrome.

### Venous Thromboembolism

Venous thromboembolism (VTE), as manifested by DVT and pulmonary embolism, is diagnosed in up to 15% of cancer patients. The overall incidence of cancer-related VTE in post-mortem studies is much higher, however, ranging between 35% and 50%. Cancer patients at greatest risk for VTE include those with mucin-secreting tumors (e.g. pancreatic and gastrointestinal cancer), cancers of the lung, brain, prostate, breast, and ovary, and patients with acute promyelocytic leukaemia and myeloproliferative disorders, specifically polycythemia vera and essential thrombocythemia. VTE often complicates the care of cancer patients undergoing major surgery and of patients receiving chemotherapy and/or hormonal therapy. The risk of developing thrombosis in cancer patients is influenced by the age and hormonal status of the patient. Postmenopausal women with advanced breast cancer receiving tamoxifen in addition to adjuvant chemotherapy have a higher risk for thrombotic events than do premenopausal women with breast cancer. Other antineoplastic agents that are associated with VTE include cisplatin, cyclophosphamide, methotrexate, and 5-fluorouracil. Thromboembolic events have been also reported with thalidomide therapy. Mechanical causes of VTE may result from vascular invasion by tumors of the kidney, stomach, lung, liver, adrenal cortex, and testicle, and form the use of central venous catheters.

Anti-platelet therapy, for example therapy utilizing the agents of the present disclosure, may be useful in the prevention of strokes, thrombosis, myocardial infarction and vascular death in a patient suffering from cancer. Such therapy may be acute therapy immediately or shortly following a cardiovascular event or, alternatively a chronic or long-term therapy to treat, prevent or reduce the likelihood of vascular disease in patients suffering from cancer and/or being treated for cancer.

VTE may be diagnosed using several techniques including determining the present probability of VTE by measuring of plasma D-dimer. Other techniques include diagnostic imaging modalities for DVT such as ascending contrast venography, compression ultrasonography, impedance plethysmography, and magnetic resonance venography. Such diagnostic techniques may be used to determine whether a patient suffering from cancer would benefit form treatment using an agent of the present disclosure.

The major concern about treatment of VTE in cancer patients is the higher risk of bleeding and of VTE recurrence compared to noncancer patients. This is particularly pertinent in patients suffering from metastases of the brain. Agents of the present disclosure, for example, but in no way limited to, a soluble GPVI protein, may be considered to cause lower amounts of bleeding than some conventional anti-coagulant drugs, and therefore may be particularly useful in treating VTE in cancer patients. Agents of the present disclosure, for example but not limited to a soluble GPVI protein or an anti GPVI single domain antibody, may be particularly useful in treating VTE in patients suffering from brain metastases, since agents of the disclosure may be considered not to significantly increase bleeding, unlike other anti-coagulant therapies.

Furthermore, cancer chemotherapies can cause significant changes in platelet counts and marked fluctuation in anticoagulant responses, which may further increase the risk for bleeding. An agent of the disclosure, for example a soluble GPVI protein, may not be affected by changes in platelet counts and therefore may be useful in treating VTE in patients being treated by chemotherapy.

Treatment of VTE using the methods, agents and products of the present disclosure described herein may be considered to include treatment of recurrent VTE in cancer patients. It is also considered that agents of the disclosure can be used to coat catheters, for example although not limited to, long-term central venous catheters which are necessary for some cancer patients, thus preventing or reducing the risk of thrombotic complications as a result of catheter use. It is considered that use of agents of the disclosure in such a method may further comprise any other of the features described herein.

Agents of the disclosure may also be used prophylactically to treat cancer patients who require long-term catheter use to reduce or prevent thrombotic and/or cardiovascular complications of catheter use.

Anti-GPVI and/or anti-fibronectin and/or anti-vitronectin treatment in accordance with the present disclosure may be used to provide clear benefit for patients with cardiovascular disease, especially those who have undergone corrective vessel surgery or angioplasties with or without stenting. Anti-GPVI, and/or anti-fibronectin and/or vitronectin treatment may be given by injection (e.g. intravenously) or by local delivery methods (e.g. pre-coating of stents or other indwelling devices).

Anti-GPVI, and/or anti-fibronectin and/or vitronectin may be delivered by gene-mediated technologies. Alternative formulation strategies may provide preparations suitable for oral or suppository route. The route of administration may be determined by the physicochemical characteristics of the treatment, by special considerations for the disease, to optimise efficacy or to minimise side-effects. Thus, the agents of the disclosures may be used to treat and/or protect against a variety of disorders, including, for example, seizures, transient ischemic shock, strokes, focal ischemia originating from thrombus or cerebral hemorrhage, global ischemia originating from cardiac arrest, trauma, neonatal palsy, hypovolemic shock, and hyperglycemia and associated neuropathies.

It has been shown that both vitronectin and fibronectin are involved in cell migration and adhesion. It has been suggested that fibronectin is involved in the metastasis of cancer cells. Similarly, it has been suggested that vitronectin may promote the differentiation of both normal and neoplastic cells. It is therefore considered that the agents of the present disclosure may be useful in treating or preventing metastasis in a subject. Thus, it is considered that agents of the present disclosure may be useful in the treatment of inflammatory disorders, such as rheumatoid arthritis and psoriasis, and cardiovascular diseases, such as atherosclerosis and restenosis. The agents of the present disclosure may be useful for the treatment or prevention of other diseases including, but not limited to, thromboembolic disorders, asthma, allergies, adult respiratory distress syndrome, graft versus host disease, organ transplant rejection, septic shock, eczema, contact dermatitis, inflammatory bowel disease, and other autoimmune diseases. The agents of the present disclosure may also be useful for wound healing. The products of the present disclosure are also useful for the treatment, including prevention, of angiogenic disorders. The term angiogenic disorders as used herein includes conditions involving abnormal neovascularization. Where the growth of new blood vessels is the cause of, or contributes to, the pathology associated with a disease, inhibition of angiogenisis will reduce the deleterious effects of the disease. An example of such a disease target is diabetic retinopathy. Where the growth of new blood vessels is required to support growth of a deleterious tissue, inhibition of angiogenisis will reduce the blood supply to the tissue and thereby contribute to reduction in tissue mass based on blood supply requirements. Examples include growth of tumors where neovascularization is a continual requirement in order that the tumor grow and the establishment of solid tumor metastases. Thus, the agents of the present disclosure inhibit tumor tissue angiogenesis, thereby preventing tumor metastasis and tumor growth. Thus, according to methods of the present disclosure, the inhibition of angiogenesis using the agents of the present disclosure can ameliorate the symptoms of the disease, and, in some cases, can cure the disease.

The present inventive method includes the administration to an animal, such as a mammal, particularly a human, in need of the inhibition of platelet activation of an effective amount, e.g., a therapeutical effective amount, of one or more of the aforementioned present inventive agents, alone or in combination with one or more other pharmaceutically active agents.

The agents of the disclosure may be administered orally, intravenously, subcutaneously, buccally, rectally, dermally, nasally, tracheally, bronchially, by any other parenteral route, as an oral or nasal spray or via inhalation, The agents may be administered in the form of pharmaceutical preparations in a pharmaceutical acceptable dosage form. Depending upon the disorder and patient to be treated and the route of administration, the compositions may be administered at varying doses.

If an agent of the disclosure is to be administered to an individual, it is particularly at least 80% pure, more preferably it is at least 90% pure, even more preferably it is at least 95% pure and free of pyrogens and other contaminants. In this context, the percent purity is calculated as a weight percent of the total protein content of the preparation, and does not include constituents which are deliberately added to the composition after the agent is purified. The present disclosure provides a method of treatment of a human or animal body in need of treatment or prevention of acute or chronic vascular diseases associated with intraarterial and/or intravenous thrombosis, which comprises administration to a human or animal of a pharmaceutically effective amount of an inhibitor or agent of the disclosure. If the agent is used as a medicament, the dosage may be in the range of from 0.1 to 100 mg/patient/day. In one embodiment the agents are used as lyophilised powders solubilised in PBS/succrose/mannitol-buffer prior to parenteral administration.

The most preferred routes of administration are injection and infusion, especially intravenous administration.

In yet another aspect, the disclosure provides a method for treating a disease or disorder selected from therapeutic or prophylactic cardiovascular conditions, thrombosis, heart attack, stroke, Intermittent coagulation, conditions with disseminated intravascular coagulation, thrombocytopenic purpura, haemolytic uraemic syndrome, damage to blood vessel wall resulting from surgery or therapy, collagen-induced inflammation, homozygous sickle disease, kidney damage by platelet and fibrin disposition on the glomerular member and micro-angiopathic vasculitides comprising administering an agent of the disclosure, to a subject with the disease or disorder or at risk of developing the disease or disorder. The treatment may be therapeutic and/or prophylactic.

Consequently, the disclosure provides for the use of an agent of the disclosure for the manufacture of a medicament to treat or prevent of a disease or disorder selected from cardiovascular conditions, thrombosis, heart attack, stroke, intermittent coagulation, conditions with disseminated intravascular coagulation, thrombocytopenic purpura, haemolytic uraemic syndrome, damage to blood vessel wall resulting from surgery or therapy, collagen-induced inflammation, homozygous sickle disease, kidney damage by platelet and fibrin disposition on the glomerular member and micro-angiopathic vasculitides.

Agents of the disclosure are advantageously used as drugs for the treatment and prevention of atherosclerosis. The agents also solve the problem of treatment of atherosclerosis by inhibition of platelet secretion.

In one embodiment, the products or agents of this disclosure are administered prophylactically.

It will be appreciated from the above that the disclosure provides inhibitors of GPVI-fibronectin interactions, which in embodiments are selective GPVI-fibronectin interaction inhibitors. It will also be appreciated from the above that the disclosure provides inhibitors of GPVI-vitronectin.

The products of the invention may also be combined and/or co-administered with any antithrombotic agent with a different mechanism of action, such as the inhibitors of thrombin or other coagulation enzymes (e.g. Factor IXa or X), antiplatelet agents acetylsalicylic acid, ticlopidine, clopidogrel, thromboxane receptor and/or synthetase inhibitors, matrix metalloprotease inhibitors, fibrinogen receptor antagonists, prostacyclin mimetics and phosphodiesterase inhibitors and ADP-receptor (Pa₂T) antagonists.

The agents of the invention may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The products of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic, an angiotensin receptor blocker and an ACE (angiotensin converting enzyme) inhibitor.

The products of the disclosure may be combined and/or co-administered with a cardioprotectant, for example an adenosine A1 or A3 receptor agonist.

There is also provided a method for treating a cardiovascular disease in a patient that comprises treating the patient with a product of the disclosure and an NSAID, e.g., a COX-2 inhibitor. Accordingly, the products of the disclosure may be combined and/or co-administered with an NSAID.

In one embodiment of the present invention, the agents of the present disclosure may be combined more than one other active agents. In one embodiment, the agents may be combined with anti-platelets drugs and/or anti-coagulant drugs. Thus, included in the present invention are methods of treatment of vascular diseases which comprise administering a combination of anti-platelet drugs and/or anti-coagulant drugs to a patient, wherein the administration may be sequential or simultaneous.

As disclosed above, any of the agents disclosed herein may be used in combination with other drugs, for example anti-thrombotic or anti-platelet drugs, in the treatment of any of the disorders disclosed herein, for example cardiovascular disorders. The agent and the drug may be for simultaneous, sequential or separate administration to a patient. That is to say that administration of the agent and other drug(s) may be separately, sequentially or simultaneously.

In the present invention a dosage form for administration of the agent and the drug “simultaneously” is not particularly limited provided that the agent and the other drug can be administered almost at the same time. One dosage form is a single unit dosage form containing the agent and the drug.

In the present invention a dosage form for administration of the agents and the drug “separately or sequentially” is not particularly limited provided that the two agents can be separately administered wherein there is a time interval between the first administration, of the agent, and the second administration, of the other drug. For example, after a pre-determined time from the first administration of the agent, the drug is administered, or after pre-determined time from the first administration of the agent, the drug is administered.

It has been shown that triple antiplatelet therapy—aspirin plus cilostazol and clopidogrel or ticiopidine—reduces the early risk of thrombotic complications after coronary stenting with a bare metal stent compared with dual therapy. The increased efficacy is not associated with an increased risk of major bleeding (J Am Coll Cardiol 2005; 46:1833-1837.)

Thus, in one embodiment, the treatment may comprise triple antiplatelet therapy comprising treating the patient with a composition comprising an agent of the present disclosure together with at least three agents, the agent all having different mods of antiplatelet activity, each being selected from the group consisting of aspirin, cilostazol, clopidogrel and ticlopidine.

Triple anti-platelet therapy as disclosed herein may include administering compositions containing the agents of the disclosure with other anti-platelet or, for example, to reduce the early risk of thrombotic complications after coronary surgery, for example stenting.

In one embodiment, the agents of the present disclosure may be administered with a drug which acts by way of a different mechanism to the agent of the present disclosure. For example, cilostazol, which selectively inhibits phosphodiesterase III, may be used in combination with an agent of the present disclosure. Alternatively or in addition, a patient may be treated with a combination of drugs including the agents of the invention and clopidogrel or ticlopidine, which are adenosine diphosphate receptor antagonists.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

According to a further aspect there is provided a parenteral formulation including a product or an agent as described herein. The formulation may consist of the product alone or it may contain additional components, in particular the product may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier.

The disclosed products may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a product of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would most desirably be administered at a pH of between 6.0 and 7.0 such as a pH of 6.5. The pH values mentioned in this paragraph are not critical, however, and formulations may fall outside them.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active product or products with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed products might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

One class of formulations disclosed herein comprises subcutaneous or intradermal formulations (for example formulations for injection) in which the active product (or active agent combination) is formulated into a parenteral preparation that can be injected subcutaneously or intradermally. The formulation for administration will comprise the active product and a liquid carrier.

The carrier utilized in a parenteral preparation that will be injected subcutaneously or intradermally may be an aqueous carrier (for example water, typically containing an additive e.g. an antioxidant and/or an isotonicity agent) or a nonaqueous carrier (again one or more additives may be incorporated). As a non-aqueous carrier for such parenteral preparations may be mentioned highly purified olive oil.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed products will not be stored in liquid formulation but as dry solids, in order to prolong shelf life.

For making subcutaneous implants, the active product may suitably be formulated together with one or more polymers that are gradually eroded or degraded when in use, e.g. silicone polymers, ethylene vinylacetate, polyethylene or polypropylene.

Transdermal formulations may be prepared in the form of matrices or membranes, or as fluid or viscous formulations in oil or hydrogels or as a compressed powder pellet. For transdermal patches, an adhesive which is compatible with the skin may be included, such as polyacrylate, a silicone adhesive or polyisobutylene, as well as a foil made of, e.g., polyethylene, polypropylene, ethylene vinylacetate, polyvinylchloride, polyvinylidene chloride or polyester, and a removable protective foil made from, e.g., polyester or paper coated with silicone or a fluoropolymer. For the preparation of transdermal solutions or gels, water or organic solvents or mixtures thereof may be used. Transdermal gels may furthermore contain one or more suitable gelling agents or thickeners such as silicone, tragacanth, starch or starch derivatives, cellulose or cellulose derivatives or polyacrylic acids or derivatives thereof. Transdermal formulations may also suitably contain one or more substances that enhance absorption though the skin, such as bile salts or derivatives thereof and/or phospholipids. Transdermal formulations may be prepared according to a method disclosed in, e.g., B W Barry, “Dermatological Formulations, Percutaneous Absorption”, Marcel Dekker Inc., New York—Basel, 1983, or Y W Chien, “Transdermal Controlled Systemic Medications”, Marcel Dekker Inc., New York—Basel, 1987.

Typically, therefore, the pharmaceutical products of the invention may be administered orally or parenterally (“parenterally” as used herein, refers to modes of administration which include intravenous, intramuscular, intraperitoneal, intrasternal, subcutaneous and intraarticular injection and infusion.) to a host to obtain an protease-inhibitory effect. In the case of larger animals, such as humans, the compounds may be administered alone or as compositions in combination with pharmaceutically acceptable diluents, excipients or carriers.

According to a further aspect of the invention there is thus provided a pharmaceutical composition including a described product, in admixture with a pharmaceutically acceptable adjuvant, diluent or carrier.

Pharmaceutical compositions of this invention for parenteral injection or infusion, e.g. intravenous injection or infusion, suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

As solvents, co-solvents or additives for parenteral, e.g. Intravenous, or other formulations may be mentioned:

Acids, e.g. with pH greater than 1.8
Bases, e.g. with pH less than 14
Cremophor EL, e.g. up to 25% in water
Dextrose, e.g. up to 5%, in water or NaCl
Ethanol, e.g. up to 15% in water
Glycerol
sorbitol
Phosphate buffer
Polyethylene glycol 300 or 400, neat or in water
Propylene glycol, neat or in water
Saline, 0.9% (or other aqueous salt solution)
poloxamer
Solutol, e.g. up to 30% in water
Tween surfactants, e.g. up to 2%
Water.

Also to be mentioned are microsphere-based delivery systems composed of the desired bioactive molecule incorporated into a matrix of poly-DL-lactide-co-glycolide (PLG).

As a further option may be mentioned lipophilic carbohydrate excipients, termed oligosaccharide ester derivatives. (OEDs), which have been used to modify pharmacokinetic profiles of drugs (SoliDose™ technology, Elan Pharmaceuticals). This technology offers the ability to formulate drug molecules with modified-release characteristics and improved bioavailability. Another technology from the same company makes use of select carbohydrate excipients, such as trehalose and sucrose to stabilize molecules in the dry state, thereby preventing their physical and chemical degradation at ambient temperatures and above.

Intravenous and other parenteral compositions may be provided as ready-to-use solutions or as lyophilisates or dry powders for reconstitution prior to administration.

Parenteral and other compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic add. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid, for example; b) binders such as carboxymethylcellulose, alginates, gelatin, polyvinylpyrrolidone, sucrose and acacia, for example; c) humectants such as glycerol, for example; d) disintegrating agents such as agar-agar, calcium carbonate, potato or tapioca starch, alginic acid, certain silicates and sodium carbonate, for example; e) solution retarding agents such as paraffin, for example; f) absorption accelerators such as quaternary ammonium compounds, for example; g) wetting agents such as cetyl alcohol and glycerol monostearate, for example; h) absorbents such as kaolin and bentonite day for example and i) lubricants such as talc, calcium stearate, magnesium stearate, solid polyethylene glycols, sodium lauryl sulfate and mixtures thereof, for example. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkyloiamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, capsules, pills, and granules can be prepared with coatings and shells such as enteric coatings and other coatings such as multiple coatings, for example, well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions which can be used include polymeric substances and waxes.

The active compounds may also be in micro-encapsulated form, if appropriate, with one or more of the above-mentioned excipients.

The active compounds may be in finely divided form, for example it may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Agents can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of an agent invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Examples of dosage forms and formulations are illustrated below in the following paragraphs. It will be understood that these are examples and do not represent limitations to the scope of the present disclosure.

1. A liquid dosage form for parenteral administration, for example a solution, suspension, liposome formulation, or emulsion in oily or aqueous vehicles. In addition to the active compounds, the liquid dosage forms may contain e.g. tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

2. A large volume parenteral (LVP), e.g. more than 100 ml of a liquid formulation, more particularly about 250 ml, of a liquid formulation of the active compound. Examples of LVPs are infusion bags.

3. A small volume parenteral (SVP), e.g. about 100 ml or less of a liquid formulation of the active compound. Examples of SVPs are vials with solution, vials for reconstitution, prefilled syringes for injection and dual chamber syringe devices.

4. A formulation having a pH of from 5 to 8 of from 6.0 to 7.0 such as a pH of 6.5 for example.

5. Parenteral formulations in glass containers. If desired, amber glass employing iron oxide or an opaque cover fitted over the container may afford the appropriate UV protection.

6. Parenteral formulations in plastics containers such as plastics infusion bags, for example.

7. Dry solid formulations for reconstitution, particularly a finely divided form such as, for example, a lyophilisate, for intravenous or other parenteral use. Solid dosage forms for parenteral administration may encompass solid and semi-solid forms and may include pellets, powders, granules, patches, and gels.

8. Subcutaneous or intradermal formulations (for example formulations for injection) in which the active product (or active agent combination) is formulated into a parenteral preparation that can be injected subcutaneously or intradermally. The formulation for administration will comprise the active product and a liquid carrier.

9. A parenteral preparation having an aqueous carrier (for example water, typically containing an additive e.g. an antioxidant and/or an isotonicity agent), for example solutions, dispersions, suspensions or emulsions.

10. A parenteral formulation having a nonaqueous carrier (again one or more additives may be incorporated), for example solutions, dispersions, suspensions or emulsions. Pharmaceutically acceptable non-aqueous carriers can be fully saturated, or partially or fully unsaturated. Examples of non-aqueous carriers include, but are not limited to:
(i) Vegetable oils (such as cottonseed oil, corn oil, sesame oil, soybean oil, olive oil, fractionated coconut oils, peanut oil, sunflower oil, safflower oil, almond oil, avocado oil, palm oil, palm kernel oil, babassu oil, beechnut oil, linseed oil, rape oil, and the like), mineral oils, synthetic oils, and combinations thereof.
(ii) Fully saturated non-aqueous carriers, examples of which include, but are not limited to, medium to large chain fatty acids (e.g. capric acid and/or caprylic acid) and particularly esters thereof (such as fatty acid triglycerides with a chain length of about 6 C to about 24 C); mixtures of fatty acids are split from the natural oil (for example coconut oil palm kernel oil, babassu oil, or the like) and are refined. In some embodiments, about 8 C to about 12 C fatty acid medium chain triglycerides are useful. Other fully saturated non-aqueous carriers include, but are not limited to, saturated coconut oil (which typically includes a mixture of lauric, myristic, palmitic, capric and capric acids), including those sold under the Miglyo trademark from Huls and bearing trade designations 810, 812, 829, and 840). Also noted are the NeoBee products sold by Drew Chemicals. Isopropyl myristate is another example of a non-aqueous carrier.
(iii) Synthetic oils, examples of which include triglycerides, and propylene glycol diesters of saturated or unsaturated fatty acids having from 6 to 24 carbon atoms such as, for example hexanoic acid, octanoic (caprylic), nonanoic (pelargonic), decanoic (capric), undecanoic, lauric, tridecanoic, tetradecanoic (myristic), pentadecenoic, hexadecanoic (palmitic), heptadecanoic, octadecanoic (stearic), nonadecanoic, heptadecanoic, eicosanoic, heneicosanoic, docosanoic, and lignoceric acids, and the like.
(iv) Unsaturated carboxylic acids, examples of which include oleic, linoleic, and linolenic acids, and the like.
(v) A “non-oil”, for example polyethylene glycol.
It will be understood that the non-aqueous carrier can comprise the mono-, di-, and triglyceryl esters of fatty acids or mixed glycerides and/or propylene glycol diesters wherein at least one molecule of glycerol has been esterified with fatty acids of varying carbon atom length

11. Sterile powders for reconstitution into sterile injectable or infusable solutions, dispersions, suspensions or emulsions prior to use, for example immediately prior to use. The injectable formulation may be in an aqueous carrier or a non-aqueous carrier.

12. Formulations comprising as aqueous and nonaqueous carriers, diluents, solvents or vehicles water, ethanol, polyols (such as glycerol, propylene glycol, polyethylene glycol, for example), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. The features of this paragraph may be applied to the formulations of any of preceding paragraphs 1, 2, 3, 4, 5, 6, 8, 9, 10 and 11.

13. Formulations comprising an acid, e.g. formulations with pH greater than 1.8, for example at least 2, e.g. at least 3, as in the case of formulations having a pH of at least 4. Often acidic formulations have a pH of at least 5.

14. Formulations comprising a base, e.g. formulations with pH of less than 14 for example less than 13, e.g. no more than 12, as in the case of formulations having a pH of no more than 11. Often acidic formulations have a pH of no more than 10, particularly no more than 9. In particular embodiments, the pH is no more than 8.

15. Formulations comprising Cremophor EL®, suitably in an aqueous carrier, e.g. up to 25% in water. Cremophor EL is also known as Polyoxyl 35 Castor oil. Cremophor EL is a non-ionic solubilizer and emulsifier obtained by causing ethylene oxide to react with castor oil of German Pharmacopoeia (DAB 8) quality in a molar ratio of 35 moles to 1 mole. Cremophor EL forms clear solutions in water. It is also soluble in ethyl alcohol, n-propyl alcohol, isopropyl alcohol, ethyl acetate, chloroform, carbon tetrachloride, trichloroethylene, toluene and xylene.

16. Formulations comprising dextrose, e.g. up to 5%, in an aqueous solvent, for example water or saline.

17. Formulations comprising ethanol, e.g. up to 15% and optionally up to 5%, in an aqueous solvent, for example water or saline.

18. Formulations comprising glycerol, e.g. in an alcoholic or aqueous solvent, for example water or saline.

19. Formulations comprising sorbitol, e.g. in an alcoholic or aqueous solvent, for example water or saline.

20. Formulations comprising phosphate buffer.

21. Formulations comprising polyethylene glycol, e.g. PEG 300 or 400, neat or e.g. in an alcoholic or aqueous solvent, for example water or saline.

22. Formulations comprising propylene glycol or a propylene glycol derivative, for example propylene glycol alginate, neat or e.g. In an alcoholic or aqueous solvent, for example water or saline.

23. Formulations comprising a sugar, e.g. lactose, sucrose or glucose, whether as a solid or in solution, e.g. in an alcoholic or aqueous solvent, for example water or saline.

24. Formulations comprising an antioxidant, e.g. In an alcoholic or aqueous solvent, for example water or saline.

25. Formulations comprising an amino acid additive, e.g. In an alcoholic or aqueous solvent, for example water or saline.

26. Formulations comprising a lipid, e.g. a phospholipid.

27. Formulations comprising saline.

28. Formulations comprising a polyoxyethylenesorbitan ester surfactant, e.g. Tween 20 (polyoxyethylenesorbitan monolaurate), Tween 40 (polyoxyethylenesorbitan monopalmitate), Tween 60 (polyoxyethylenesorbitan monostearate), Tween 80 (polyoxyethylenesorbitan monooleate) or Tween 85 (polyoxyethylenesorbitan trioleate).

29. Formulations comprising a poloxamer (poly(oxyethylene)-poly(oxypropylene) block copolymer).

30. Formulations comprising a Solutol, for example Solutol HS 15 (Polyethylene glycol-15-hydroxystearate), e.g. up to 30% in water

31. Formulations comprising a microsphere-based delivery system, e.g. composed of the desired bioactive molecule incorporated into a matrix of poly-DL-lactide-co-glycolide (PLG).

32. Formulations comprising lipophilic carbohydrate excipients, termed oligosaccharide ester derivatives (OEDs), which have been used to modify pharmacokinetic profiles of drugs (SoliDose™ technology, Eian Pharmaceuticals).

33. Intravenous and other parenteral compositions provided as ready-to-use solutions, suspensions, liposome formulations, or emulsions in oily or aqueous vehicles

34. Intravenous and other parenteral compositions provided as lyophilisates or dry powders for reconstitution prior to administration.

35. A subcutaneous implant.

36. A transdermal formulation, for example in the form of matrices or membranes, or as fluid or viscous formulations in oil or hydrogels or as a compressed powder pellet. See above for further details.

37. Injectable depot forms, e.g. comprising microencapsule matrices of the drug in biodegradable polymers, for example a polylactide-polyglycolide, poly(orthoester) and poly(anhydride), or comprising the drug entrapped in liposomes or microemulsions which are compatible with body tissues.

38. Formulations comprising a biodegradable polymer, e.g. one mentioned previously.

It will be appreciated that dosage forms and formulations of the present invention may be one of, or any permissible combination of, features(s) of any of the above paragraphs 1 to 38.

Advantageously, the compounds of the invention are orally active, have rapid onset of activity and low toxicity.

The compounds of the invention have the advantage that they may be more efficacious, be less toxic, be longer acting, have a broader range of activity, be more potent, produce fewer side effects, be more easily absorbed than, or that they may have other useful pharmacological properties over, compounds known in the prior art.

The invention also includes a method for the long term or chronic prevention of atherosclerosis and/or arterial thrombosis, comprising administering to a subject an effective amount of an agent of the disclosure.

Example 8 below contains data indicating that the agents disclosed herein will prevent or retard atherogenesis, in patients without active atherosclerotic lesions (or at least substantially free of observable lesions) as well as in patients with active atherosclerotic lesions. The data indicate that the agents will be useful to treat or prevent vascular disorders caused by or associated with dysfunctional endothelium (e.g. atherogenesis, ischemia, pro-thrombotic state, thrombosis, pro-inflammatory state, inflammation, atherosclerotic lesion formation, plaque activation and rupture, vasospasm, impaired vasomotion). The data further indicate that the agents will be useful for the treatment or prevention of stroke, myocardial infarction and atherosclerosis as well as other cardiovascular diseases.

Included in the disclosure therefore are methods for performing the following treatments by administering a therapeutically effective amount of an agent disclosed herein:
treatment, e.g. prevention or retardation of, atherogenesis in patients or arterial sites without active atherosclerotic lesions
treatment, e.g. prevention or retardation of, atherogenesis in patients or arterial sites with active atherosclerotic lesions
treatment, e.g. prevention or retardation of, atheroprogression in patients or arterial sites without active atherosclerotic lesions
treatment, e.g. prevention or retardation of, atheroprogression in patients or arterial sites with active atherosclerotic lesions
treatment, e.g. prevention or retardation of, vascular disorders caused by or associated with dysfunctional endothelium in patients or arterial sites without active atherosclerotic lesions
treatment, e.g. prevention or retardation of, vascular disorders caused by or associated with dysfunctional endothelium in patients or arterial sites with active atherosclerotic lesions
treatment, e.g. prevention or retardation of, stroke, myocardial infarction and atherosclerosis as well as other cardiovascular diseases in patients or arterial sites without active atherosclerotic lesions
treatment, e.g. prevention or retardation of, stroke, myocardial infarction and atherosclerosis as well as other cardiovascular diseases in patients or arterial sites with active atherosclerotic lesions
treatment, e.g. prevention or retardation of, vascular disorders in patients or arterial sites with dysfunctional endothelium
treatment, e.g. prevention or retardation of, vascular disorders in patients or arterial sites without dysfunctional endothelium.

Further included is the use of the agents described herein for the manufacture of medicaments for use in the above treatments.

Methods of treatment described herein may be chronic or acute. In the case of chronic treatments, the agents described herein may be administered over a period of at least one month, e.g. two, four, six or twelve months or more. Advantageously, chronic treatment is performed by oral administration, for example of an oral formulation as described herein. Amongst oral formulations are those containing humanised antibodies (e.g. humanised Fabs) and single domain antibodies, e.g. VHH's. Acute treatment may be administered parenterally, e.g. by intravenous injection and/or infusion.

Particular formulations, whether oral or parenteral, comprise an active agent which binds to vitronectin or fibronectin (or, and this is more usual, both) and blocks (that is, blocks to a therapeutically useful extent) binding of the vitronectin or fibronectin to GPVI, thereby inhibiting platelet/endothelium adhesion, including amongst others in patients or arterial sites substantially free of observable active atherosclerotic lesions. Exemplary are polypeptides, e.g. proteins, comprising the extracellular domain of GPVI or a vitronectin-binding or fibronectin-binding amino acid sequence contained within said domain; in either case a natural sequence may be modified by one or more amino acid deletions, substitutions or insertions (e.g. conservative substitutions). Please see earlier in this specification for descriptions of variants of natural sequences, particularly those derived from GPVI, and polypeptides having the vitronectin-binding and/or fibronectin-binding characteristics of GPVI (but not necessarily its quantitative degree of affinity for vitronectin or, as the case may be, fibronectin, or both). In one embodiment, there is provided use of a GPVI-Fc fusion protein having the sequence disclosed in FIG. 7 of WO03/104282 (PCT Application No. PCT/EP03/05929) in the uses and treatments disclosed above.

### Assays

The activity of an agent of the present disclosure can also be analyzed by a platelet adhesion assay. Briefly, the adhesion assay is performed as follows: 5′Cr-labeled platelets are incubated in microtiter plates that have collagen, convuixin or BSA immobilized to the surface of the wells, the cells are washed, 2% SDS is added to each well, and the number of adherent platelets is determined by counts for 5′Cr using a scintillation counter (see, e.g., Jandrot-Perrus et al., 1997, J. of Biol. Chem. 272: 27035-27041). Further, the activity of an agent can be analyzed by platelet aggregation assays or secretion assays known to those of skill in the art (see, e.g., Morol et al., 1989, J. Clin. Invest. 84: 1440-1445 and Poole et al., 1997, EMBO J. 16 (9): 2333-2341). Briefly, the platelet aggregation is performed as follows: platelets are incubated with collagen or convuixin in a cuvette at 37 C while being stirred, and the cell suspension is monitored by a lumiaggregometer.

Such assays may be utilized as part of GPVI, collagen, fibronectin or vitronectin diagnostic assays. In addition, such assays may be utilized as part of screening methods for identifying compounds that modulate the activity and/or expression and/or interactions of GPVI, collagen, fibronectin or vitronectin.

The disclosure includes a method (also referred to herein as a “screening assay”) for identifying agents, i. e., candidate or test compounds or agents (e.g., peptides, peptidomimetics, small molecules or other drugs) which bind to polypeptides of the present disclosure or have a stimulatory or inhibitory effect on, for example, expression or activity of a polypeptide for example, GPVI, collagen, fibronectin or vitronectin. Thus, the disclosure provides a method of identifying an agent which binds to GPVI, fibronectin and/or vitronectin comprising contacting a candidate agent with GPVI, fibronectin and/or vitronectin or fragments thereof.

In one embodiment, there are provided assays for screening candidate or test compounds which bind to or modulate the activity of the membrane-bound form of polypeptides, for example GPVI, fibronectin or vitronectin, or biologically active portions thereof. The agents of the present disclosure can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12: 145). Examples of methods for the synthesis of molecular libraries can be found in the art, for example in: DeWitt et al. (1993) Proc. Natl. Acad. Sci. USA 90: 6909; Erb et al. (1994) Proc. Natl. Acad. Sci. USA 91: 11422; Zuckermann et al. (1994). 3 Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and Gallop et al. (1994) J. Med. Chem. 37: 1233. Libraries of compounds may be presented in solution (e.g., Houghten (1992) Bio/Techniques 13:412-421), or on beads (Lam (1991) Nature 354: 82-84), chips (Fodor (1993) Nature 364: 555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89: 1865-1869) or phage (Scott and Smith (1990) Science 249: 386-390; Devlin (1990) Science 249: 404-406; Cwirla et al. (1990) Proc. Natl. Acad. Sci. USA 87: 6378-6382; and Felici (1991) J. Mol. Biol. 222:301-310). Other methods for use in screening assays which are known in the art are included in the present disclosure.

In one embodiment, an assay is a cell-based assay in which a cell which expresses a membrane-bound form of polypeptides of the disclosure, for example GPVI, fibronectin and/or vitronectin, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with 125I, 35S, 14C, or 3H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of the polypeptides, for example GPVI, fibronectin and/or vitronectin, or a biologically active portion thereof, on the cell surface with a known compound which binds the polypeptide to form an assay mixture, contacting the assay mixture with a test compound, and determining the ability of the test compound to interact with GPVI, fibronectin and/or vitronectin, wherein determining the ability of the test compound to interact with GPVI, fibronectin and/or vitronectin comprises determining the ability of the test compound to preferentially bind to GPVI, fibronectin and/or vitronectin or a biologically active portion thereof as compared to the known compound.

In another embodiment, an assay is a cell-based assay comprising contacting a cell expressing a membrane-bound form of the polypeptides, for example GPVI, fibronectin and/or vitronectin, or a biologically active portion thereof, on the cell surface with a test compound and determining the ability of the test compound to modulate (e.g., stimulate or inhibit) the activity of the polypeptide or biologically active portion thereof or to interfere with its interaction with other molecules. Determining the ability of the test compound to modulate the activity of GPVI, fibronectin and/or vitronectin or a biologically active portion thereof can be accomplished, for example, by determining the ability of the polypeptide protein to bind to or interact with a target molecule. Determining the ability of a polypeptide of the disclosure to bind to or interact with a target molecule can be accomplished by one of the methods described above for determining direct binding. As used herein, a “target molecule” is a molecule with which a selected polypeptide (e.g., GPVI, fibronectin and/or vitronectin) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide described herein or some other polypeptide or protein. For example, a target molecule can be a component of a signal transduction pathway which facilitates transduction of an extracellular signal (e.g., a signal generated by binding of a compound to a polypeptide of the disclosure) through the cell membrane and into the cell or a second intercellular protein which has catalytic activity or a protein which facilitates the association of downstream signaling molecules with GPVI, fibronectin and/or vitronectin.

Determining the ability of the polypeptide, for example, GPVI, collagen, fibronectin and/or vitronectin, to bind to or interact with a target molecule can be accomplished by determining the activity of the target molecule. For example, the activity of the target molecule can be determined by detecting induction of a cellular second messenger of the target (e.g., intracellular Ca2+, diacylglycerol, IP3, etc.), detecting catalytic/enzymatic activity of the target on an appropriate substrate, detecting the induction of a reporter gene (e.g., a regulatory element that is responsive to a polypeptide of the disclosure operably linked to a nucleic acid encoding a detectable marker, e.g., luciferase), or detecting a cellular response, for example, cellular differentiation, or cell proliferation.

In yet another embodiment, an assay of the present disclosure is a cell-free assay comprising contacting GPVI, collagen, fibronectin and/or vitronectin or biologically active portion thereof with a test compound and determining the ability of the test compound to bind to GPVI, collagen, fibronectin and/or vitronectin or biologically active portion thereof. Binding of the test compound to GPVI, collagen, fibronectin and/or vitronectin can be determined either directly or indirectly as described above. In a preferred embodiment, the assay includes contacting GPVI, collagen, fibronectin and/or vitronectin or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the disclosure for example GPVI, collagen, fibronectin and/or vitronectin or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the disclosure to further modulate the target molecule. For example, the catalytic/enzymatic activity of the target molecule on an appropriate substrate can be determined as previously described.

In yet another embodiment, the cell-free assay comprises contacting a polypeptide of the disclosure, for example GPVI, vitronectin, collagen and/or fibronectin or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present disclosure are amenable to use of both a soluble form or the membrane-bound form of a polypeptide of the disclosure, for example GPVI, collagen, vitronectin and/or fibronectin. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly (ethylene glycol ether) n,3-[(3-cholamidopropyl)dimethylamminio]-I-propane sulfonate (CHAPS),3-[(3-cholamidopropyl) dimethylamminlo]-2-hydroxy-1-propane sulfonate (CHAPSO), or N-dodecyl=N,N-dimethyl-3-ammonio-I-propane sulfonate. In more than one embodiment of the above assay methods of the present disclosure, it may be desirable to immobilize either the polypeptide of the disclosure or its target molecule to facilitate separation of complexed from uncomplexed forms of one or both of the proteins, as well as to accommodate automation of the assay. Binding of a test compound, to consider whether it is an agent according to the present disclosure, to the polypeptide, or interaction of the polypeptide with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-5-transferase fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a polypeptide of the disclosure, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the disclosure can be determined using standard techniques. Other techniques for immobilizing proteins on matrices can also be used in the screening assays of the disclosure. For example, either the polypeptide of the disclosure, for example GPVI, collagen, vitronectin and/or fibronectin or its target molecule can be immobilized utilizing conjugation of biotin and streptavidin.

Biotinylated polypeptide of the disclosure or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptides of the disclosure or target molecules but which do not interfere with binding of the polypeptide of the disclosure to its target molecule can be derivatized to the wells of the plate, and unbound target or polypeptide of the disclosure trapped in the wells by antibody conjugation. Methods for detecting such complexes, in addition to those described above for the GST-Immobilized complexes, include immunodetection of complexes using antibodies reactive with the polypeptide(s) of the disclosure or target molecule, as well as enzyme-linked assays which rely on detecting an enzymatic activity associated with the polypeptide of the disclosure, for example, GPVI, fibronectin, vitronectin and/or collagen or target molecule.

In another embodiment, modulators of expression of a polypeptide of the disclosure are identified in a method in which a cell is contacted with a candidate compound, to consider whether it is an agent of the present disclosure and the expression of the selected mRNA or protein (i. e., the mRNA or protein corresponding to a polypeptide or nucleic acid of the disclosure) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the disclosure based on this comparison. For example, when expression of the selected mRNA or protein is greater (statistically significantly greater) in the presence of the candidate compound than in its absence, the candidate compound is identified as a stimulator of the selected.

Thus, the disclosure provides for the use of fibronectin and/or vitronectin or fragments thereof in a binding assay for identifying an agent capable of binding GPVI, preferably an agent capable of inhibiting platelet aggregation, more preferably an agent capable of inhibiting platelet aggregation by fibronectin and/or vitronectin interaction with platelet-bound GPVI. Once a candidate agent has been identified which binds to GPVI then further assays can be carried out to check for the preferred activities described herein.

The present invention considers the use of fibronectin or fragments thereof having GPVI binding activity in a binding assay for identifying an agent capable of inhibiting interaction of GPVI and fibronectin. In one embodiment, the agent is capable of inhibiting binding of GPVI to fibronectin.

In one aspect of the disclosure, there is provided, the use of vitronectin or fragments thereof having GPVI binding activity in a binding assay for identifying an agent capable of inhibiting interaction of GPVI and vitronectin. The agent may be capable of inhibiting binding of GPVI to fibronectin.

### EXAMPLES

The following methods and examples represent a way of producing or obtaining an agent of the present invention. It will be apparent to the skilled person that alternative methods are available to obtain the agent of the invention.

### Example 1

### Cloning of the Fully Human Fusion Protein of GPVI (Fc-GPVI-nt)

To generate a soluble form of human GPVI, the extra-cellular domain of human GPVI was cloned and fused to the human immunoglobin Fc-domain. The Fc was amplified from a human heart cDNA library (Clonetech, Palo Alto, Calif.) by PCR using the forward primer 5′-cgcggggcggccgcgagt-ccaaatcttgtgacaaaac-3′ and the reverse primer 5′-gcgggaagctttcatttacccggagacagggag-3′. The PCR reaction was performed at 58° C. annealing temperature and 20 cycles with the Expand High Fidelity PCR System (Roche Molecular Biochemicals, Mannheim, Germany). The PCR fragment was cloned in the plasmid pADTrack CMV with NotI/HindIII and the sequence was checked by sequencing (MediGenomix, Martinsried, Germany).

For cloning of the extracellular domain of the human GPVI RNA from cultured megakaryocytes was isolated (RNeasy Mini Kit; Qiagen, Hilden, Germany) according to the manufacturer's protocol and reverse transcription was performed (Omniscript RT Kit; Qiagen) with 2 μg RNA at 37° C. overnight. 100 ng of the reaction was used as a template in PCR amplification of the hGPVI with the primer 5′-gcggggagatctaccaccatgtctccatccccgacc-3′ and 5′-cgcggggcggccgccgttgcccttggtgtagtac-3′. The PCR reaction was performed at 54° C. annealing temperature and 24 cycles with the Expand High Fidelity PCR System (Roche Molecular Biochemicals, Mannheim, Germany). The PCR fragment was cloned in the plasmid pADTrack CMV Fc with BglII/NotI and the sequence was checked by sequencing.

### Example 2

### Cloning of Stable Fc-GPVI-nt-CHO-Fp-n Cells for Expression and Secretion of FcGPVI-nt

The human Fc-GPVI-nt was amplified from the plasmid pADTrackCMV human Fc-GPVI-nt by PCR using the forward primer 5′-gcgggggctagcaccaccatgtctccatccccgac-3′ and the reverse primer 5′-cgcgggggatcctcatttacccggagacagggag-3′. The PCR reaction was performed at 58° C. annealing temperature and 24 cycles with the Expand High Fidelity PCR System (Roche Molecular Biochemicals, Mannheim, Germany). The PCR fragment was cloned in the plasmid pREP4 (Invitrogen, Carlsbad, Calif.) with NheI/BamHI and the sequence of the resulting plasmid pREP4 human Fc-GPVI-nt was checked by sequencing (MedlGenomix, Martinsried, Germany). CHO K1 cells (DSMZ, Braunschweig, Germany) were transfected with the plasmid pREP4 human FC-GPVI-NT using effectene transfection reagent (Qiagen, Hilden, Germany). 48 hours after transfection the cells were split in medium containing 200 μg/ml hygromycin. Single colonies were picked and the expression was tested by precipitation of the recombinant protein with Protein-A-sepharose (Amersham Pharmacia Biotech AB, Uppsala, Sweden) from 1 ml culture supernatant. After SDS-PAGE the proteins were detected with peroxidase-conjugated goat anti-human IgG antibody (Fc-fragment specific; 109-035-098; Dianova, Hamburg, Germany). Fc-GPVI-nt and control Fc were expressed as secreted soluble proteins using the CHO cell line to prevent misfolding and non-glycosylation of the expressed proteins.

### Example 3

### Generation of Monoclonal Antibodies Against Human GPVI

Monoclonal antibodies were generated essentially as described (Kremmer, E., Kranz, B. R., Hille, A., Klein, K., Eulitz, M., Hoffmann-Fezer, G., Felden, W., Herrmann, K., Delecluse, H. J., Delsol, G., Bornkamm, G. W., Mueller-Lantzsch, N., Grassert, F. A. (1995) Rat monoclonal antibodies differentiating between the Epstein-Barr virus nuclear antigens 2A (EBNA2A) and 2B (EBNA2B). Virology 208, 336-342). Lou/C rats were immunized with human dimeric Fc-GPVI-nt fusion protein (PR-15) as disclosed in WO03/104282). Screening of hybridoma supernatants was performed in a solid-phase immunoassay using (PR-15) dimeric Fc-GPVI-nt or an Fc portion lacking the GPVI domain. Screening identified the supernatant of hybridoma different antibodies to bind specifically to dimeric Fc-GPVI-nt but not to Fc lacking the external GPVI domain. The immunoglobulin type was determined with rat Ig class (anti-IgM) and IgG subclass-specific mouse mAbs. The monoclonal antibodies were purified using Protein G-Sepharose columns. Antibody specificity of monoclonal antibody (specific to GPVI) hGP 5C4 was verified by immunoblotting against dimericFc-GPVI-nt and control Fc. hGP 5C4 monoclonal antibody detected recombinant dimeric Fc-GPVI-nt but not control Fc (data not shown). Furthermore, hGP 5C4 binds specifically to the surface of human platelets (data not shown).

### Example 4

### Generation of Fab-Fragments of Monoclonal IgG Antibodies

Complete IgG antibodies were digested to generate Fab-fragments of anti-GPVI antibodies with ImmunoPure Fab Kit (Pierce Biotechnology, Inc., Rockford, Ill., USA) according to the manufacturer's instructions. Accordingly, IgG molecules were digested into Fab fragments and Fc fragments by using immobilized papain. After digestion, the fragments were purified on an immobilized Protein A column. Detailed instructions allow for flexibility in the protocol for hard to digest antibodies. The success of Fab-fragment generation was tested by comparing molecular size of both antibody formats in SDS gels and staining with Coomassie blue (data not shown).

### Example 5

Confluent human umbilical vein endothelial cells (HUVEC) were activated with TNF-α (50 ng/ml) and INF-γ (20 ng/ml) for 16 hours. Thereafter, the cells were pre-treated with a soluble form of recombinant dimeric human GPVI (100 μg/ml) fused to the human immunoglobulin Fc domain via a specific hinge region (hGPVI-Fc) (PR-15)^{11,12} or with equimolar amount of Fc control for 60 min. Recombinant soluble dimeric hGPVI-Fc exhibits high affinity for collagen and inhibits collagen-induced platelet aggregation^{11,12}.

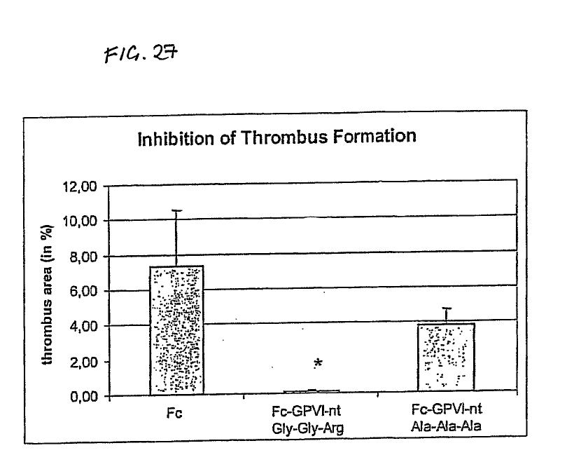

Surprisingly, perfusion of isolated human platelets for 10 min over hGPVI-Fc pre-treated HUVEC resulted in a substantial reduction (p<0.05) of platelet adhesion to endothelium compared with controls by approximately 50% (FIG. 19a) suggesting that activated endothelium exposes binding sites for GPVI. To test whether GPVI directly binds to HUVEC, TNF-α/INF-γ-activated HUVEC were incubated with hGPVI-Fc (20 μg/ml) or an equivalent amount of control Fc for 60 min and determined surface binding of both proteins by flow cytometry. A significant and specific binding of dimeric hGPVI-Fc to HUVEC was found (FIG. 19b). Thus, these data show that GPVI recognizes a binding partner on endothelial cells that mediates platelet adhesion to endothelium.

Activated endothelial cells express a variety of adhesion receptors (e.g. β₃-integrin, P-selectin, ICAM-1)¹³⁻²¹ and molecules (e.g. von Willebrand factor (vWF), vitronectin (Vn), fibronectin (Fn))^{22,24} that all have been shown to play a role in platelet/endothelium adhesion. Most of these adhesion molecules are presented in substantial amounts on the luminal aspect of endothelial cells (FIG. 19c) and thereby are potential candidates for endothelial GPVI ligands. To identify specific ligands of GPVI, binding of recombinant hGPVI to a variety of immobilized adhesion molecules (vWF, Vn, Fn) or adhesion receptors stably expressed in Chinese hamster ovary (CHO) cells (αᵥβ₃, P-selectin, ICAM-1) was directly assessed. It was found, unexpectedly, that dimeric hGPVI specifically binds to both immobilized Vn and Fn in a concentration-dependent manner (FIG. 19d). Binding of soluble GPVI to Vn or Fn was substantially inhibited in the presence of a Fab fragment of the blocking anti-GPVI mAb, hGP 5C4 (FIG. 19e). In contrast, virtually no GPVI binding to immobilized vWF was evident (FIG. 19d). To test whether GPVI binds to adhesion receptors CHO cells that stably express αᵥβ₃, P-selectin, or ICAM-1 on the cell surface were incubated with soluble GPVI or control Fc. No specific binding of hGPVI to these cells (data not shown) suggesting that neither of these receptors is recognized by GPVI. Together, these findings show that GPVI, which has been previously thought to act solely as a signal-transducing receptor for subendothelial collagen, also specifically binds to immobilized Fn or Vn, both of which are expressed on the surface of activated endothelial cells.

### Example 6

To analyze whether Vn and Fn mediate GPVI-dependent platelet adhesion under flow conditions, purified human Vn, Fn, vWF, and collagen were each immobilized on glass coverslips. After incubation of immobilized ligands with hGPVI-Fc (PR-15) or control Fc (20 μg), adhesion of isolated platelets under defined wall shear rates in a parallel plate flow chamber^{25,26} ws evaluated. Adhesion of isolated platelets to these matrices was monitored for 5 min at a wall shear rate of 2000 s⁻¹. On all ligands, robust platelet adhesion occurred within 5 min and this was not significantly altered when coverslips had been preincubated with control Fc. In contrast, pre-incubation with hGPVI-Fc (PR-15) reduced platelet adhesion to collagen, Fn, and Vn by approximately 50% whereas adhesion to vWF was almost unaffected by the treatment (FIG. 20a). The effect of soluble GPVI on platelet adhesion to Vn and Fn was not dependent on divalent cations because a similar extent of inhibition was found in the presence of EDTA (FIG. 20a). Thus, the inhibition of platelet adhesion to immobilized Vn or Fn through soluble GPVI is independent of integrin receptors.

Furthermore, CHO cells stably transfected with human GPVI¹² showed substantially enhanced adhesion to immobilized Vn and Fn (FIG. 20b). This finding demonstrates that GPVI alone is sufficient to mediate cellular adhesion on Fn and Vn and establishes a novel role of GPVI for platelet physiology.

Activation of endothelial cells induces apical expression of Vn and Fn both on cultured endothelium in vitro^{21,23} and in atherogenic diabetic mice in vivo²⁴. It has previously been demonstrated by intravital fluorescence microscopy that platelets adhere to the intact and activated endothelium of the carotid artery in apoE^{-/-} mice prior to lesion formation²⁷.

### Example 7

To assess, the role of GPVI for platelet adhesion in the early stage of atherogenesis, platelet adhesion to the carotid endothelium was studied using intravital video fluorescence microscopy in apoE^{-/-} mice that were on cholesterol-enriched diet for 6 weeks. At this stage, significant platelet adhesion occurs at lesion-prone sites, but no lesion formation is detectable by histochemistry²⁷. ApoE^{-/-} mice received fluorescence-tagged syngeneic platelets preincubated with IgG or Fab fragments of a blocking monoclonal antibody (JAQ1) that is directed against the murine GPVI receptor^{28,29}. As control, a Fab fragment of the mAb 300N/A²⁷ that is directed against murine GPIIb-IIIa (not shown) or an unspecific IgG (FIG. 20c) were tested.

It was found that adhesion of platelets to the carotid endothelium of 6-weeks old apoE^{-/-} mice was substantially reduced in the presence of JAQ1 by approximately 80% and 95%, respectively, compared with vehicle or IgG control (P<0.001) (FIG. 20c). Thus, GPVI mediates platelet adhesion in vivo to diseased endothelium of the lesion prone site during atherogenesis. To determine whether GPVI directly binds to the intact arterial wall, sections of the common carotid artery of 6-weeks old apoE^{-/-} mice were stained with either hGPVI-Fc (10 μg/ml) or control Fc. Most strikingly, it was found specific hGPVI-Fc binding to the endothelial monolayer of the common carotid artery (FIG. 20d) confirming the in vitro observation that GPVI binds to a ligand present on endothelium. Furthermore, two color immunofluorescence studies showed that binding of hGPVI co-localizes with Fn presented on the luminal surface of the atherosclerotic carotid artery (FIG. 20e). Because the present disclosure (FIG. 20d) and others²⁴ found that expression of Fn is enhanced on the apical side of endothelium in atherogenic mice, the present data strongly suggest that the interaction of GPVI with Vn or Fn presented on endothelium is of biological relevance and mediates platelet adhesion in vivo.

### Example 8

Recently it was demonstrated that platelet adhesion to endothelium is critical for atherogenesis in mice²⁷. To determine the role of GPVI-mediated platelet/endothelium adhesion for vascular remodeling in atherogenic mice, 6-weeks old apoE^{-/-} mice were treated with the anti-GPVI mAb JAQ1^{28,29} or control IgG over a period of 4 weeks by intraperitoneal injection twice a week of 50 μg. Treatment of mice with JAQ1 results in specific and sustained loss of GPVI from circulating platelets and, consequently, abolished collagen responses to those cells²⁹. The degree of lesion formation was assessed in the carotid artery and the aortic arch thereafter at the age of 10, 13 and 16 weeks, respectively. In both vascular beds, the prolonged inhibition of GPVI during the early stage of atherogenesis significantly limited lesion formation (FIGS. 21aa and 21b). Quantitative computer-assisted image analysis of the carotid artery showed an approximately 69% and 73% reduction (p<0.01) in the extent of fatty streak formation in 13- and 16-wk-old apoE^{-/-} mice, respectively, as assessed by Sudan-III staining (FIG. 21b). Similarly, albeit to a lesser degree, anti-GPVI-treated apoE^{-/-} mice showed substantial decreases in lesion formation in the aortic arch (27% and 36% reduction in 13- and 16-wk-old apoE^{-/-}, respectively, p<0.01) (FIG. 21a and 21b).

Platelet adhesion to endothelial cells plays a critical role in a variety of disease states such as reperfusion and cardiovascular diseases including stroke, myocardial infarction, and atherosclerosis^{1,2,4}. Defining the relative importance of adhesion receptors has been a central goal in order to understand the molecular requirements of platelet/endothelium adhesion. The present disclosure indicates an unexpected role of the platelet receptor GPVI for platelet-endothelium adhesion and vascular remodelling. Although initially recognized as a collagen receptor⁷ the present specification discloses for the first time the identification of identified novel ligands for GPVI that are presented on the apical surface of dysfunctional endothelium^{23,24}.

### Methods of Examples 5 to 8

Human platelets were isolated as described². In animal studies whole blood from mice was collected from the retro orbital plexus in acid citrate dextrose (150 μl per ml blood) from anesthesized mice. Washed platelets were resuspended in Tyrode's solution HEPES buffer (mmol/L: HEPES 2.5, NaCl(mmol/L: HEPES 2.5, NaCl 150, NaHCO₃ 12, KCl 2.5, MgCl₂ 1, CaCl₂ 2, and D-glucose 5.5, and 1 mg/mL BSA, pH 7.4) to obtain a final platelet count of 1×10⁸/mL.

### Flow Chamber Experiments

Human and mouse platelets were isolated as described^{2,30}. Experiments were essentially performed as described elsewhere^{12,26}. Briefly, transparent flow chambers with a slit depth of 50 μm were cultivated with HUVEC until confluency was obtained. Perfusion was performed for 10 min using a pulse-free pump under a high shear rate of 2000 s⁻¹. Thereafter, chambers were rinsed by a 4 min perfusion with Tyrode's solution-HEPES buffer at the same shear rate, and phase-contrast images were recorded from 5 to 8 different microscopic fields (20× objectives). In experiments with immobilized ligands, coverslips were coated with collagen, vWF, Vn, or Fn and isolated platelets were perfused as described above.

### Cell Culture

Primary human umbilical vein endothelial cells (HUVEC) (PromoCell, Germany) were grown in 24-well culture plates (Nunc) as described². The stable CHO cell lines were kindly provided by Dr Mark Ginsberg (αᵥβ₃) and Dr Roger McEver (P-selectin). ICAM-1-transfected and untransfected Chinese hamster ovary (CHO) cells were purchased from American Type Culture Collection (Manassas, U.S.A.). CHO-hGPVI stable cell line were generated as described¹².

### Flow Cytometry

Confluent monolayers of HUVEC were stimulated with TNF-α (50 ng/ml) and INF-γ (20 ng/ml) for 16 hours. Thereafter, HUVEC were incubated with 10 μg/ml with hGPVI-Fc or Fc, respectively. After two steps of gentle washings FITC-labeled anti-human Fc mAb was added for 30 min. Endothelial cells were detached with trypsin/EDTA and analysed on a FACScallibur (Becton Dickinson, Heidelberg, Germany). HUVEC were gated by FSC/SSC characteristics². In further experiments, non-stimulated or TNF-α/INF-γ-activated HUVEC were incubated for 30 min with fluorochrome-conjugated mAbs as indicated at saturating concentrations before flow cytometric analysis.

### GPVI Binding to Immobilized Ligands

To generate a soluble form of GPVI, the extracellular domain of hGPVI was fused to the human Fc domain as described¹¹. For assessment of hGPVI-Fc binding to immobilized ligands, ELISA plates (Immulon2 HB, Dynx Technologies, Chantilly, Va.) were coated over night at 4° C. with 1 μg collagen (type I bovine; BD Bioscience, Bedford, Mass.), 2 μg vitronectin, 2 μg fibronectin (both human, Becton Dickinson, Heidelberg, Germany), or 2 μg vWF (human, Calbiochem, Bad Soden, Germany) in 100 μl coating buffer (1.59 g/l Na₂ CO₃, 2.93 g/l NaHCO₃, 0.2 g/l NaN₃, pH 9.6). The plates were washed with PBS/0.05% Tween 20 (PBST) twice and blocked with Roti-Block (Roth, Karlsruhe, Germany) over night. Then, 3.0, 6.0, 12.5, 25.0, 50.0 or 100 μg/ml GPVI-Fc in PBST was added and the plate was incubated for 1 hr at room temperature. After washing, peroxidase-conjugated goat anti-human IgG antibody Fcγ fragment specific (Dianova, Hamburg, Germany) was added and incubated for 1 hr at room temperature. After 5 fold washing with 250 μl PBST 100 μl detection reagent (BM Blue POD Substrate; Roche, Mannheim, Germany) was added and incubated up to 10 min. The reaction was stopped by the addition of 100 μl 1 M H₂ SO₄ and the plate was measured at 450 nm against reference wavelength 690 nm. The assay was developed as described¹². A rat monoclonal antibody hGP 5C4 was prepared against the recombinant soluble human GPVI and found to inhibit collagen-induced platelet aggregation¹². Two μg of GPVI were incubated with 5 μg of Fab 5C4 for 30 min and GPVI binding to ligands was analyzed as described above. Adhesion of GPVI-transfectants to immobilized ligands was evaluated. ELISA plates (Immulon2 HB, Dynx Technologies, Chantilly, Va.) were coated over night at 4° C. with collagen, Fn, or Vn. Single separated CHO cells (CHO or CHO-GPVI¹¹) (1×10⁵ per well) were added to the wells for 30 min. After three rounds of gentle washing with Tyrode's buffer cell, adhesion was quantified using a color reaction based method¹¹.

### Animals

Four-week-old male apoE^{-/-} (C57BL/63-ApoE^{tm1Unc}) mice (The Jackson Laboratory, Bar Harbor, Me., USA) consumed a 0.25% cholesterol diet (Harlan Research diets, 0% cholate) for another 6, 9, or 12 weeks, respectively (n=5 per group).

### Video Fluorescence Microscopy

Platelet adhesion were monitored in vivo by use of video fluorescence microscopy as described in detail elsewhere^{12,30}. Where indicated, fluorescent wild type platelets were preincubated with 50 μg/ml ant-GPVI (JAQ1-Fab)²⁸ or control IgG for 10 minutes prior to infusion. The number of adherent platelets was assessed by counting the cells that did not move or detach from the endothelial surface within 20 seconds^{12,30}.

### Assessment of In Vivo GPVI-Fc Binding by Immunohistochemistry

The remaining carotid arteries obtained from mice were shock frozen and embedded in cryoblocks (medite, Medizintechnik GmbH, Burgdorf, Germany). 5 μm cryostat sections were incubated with hGPVI-Fc (10 μg/ml) or control Fc (10 μg/ml) for 60 min. After blocking and washing sections were stained with peroxidase-conjugated goat anti-human IgG antibody Fcγ fragment specific (Dianova, Hamburg, Germany). For dual color staining, sections were stained with soluble GPVI-Fc (PR-15) and anti-mouse Fn (Santa Cruz). After addition of secondary mAbs co-localization of antigens was analyzed by confocal laser microscopy.

Chronic Inhibition of GPVI in apoE^{-/-} Mice

To determine the relevance of GPVI for atheroprogression, 6-wk-old apoE^{-/-} mice were randomly assigned to receive either an irrelevant rat immunoglobulin (50 μg, n=5) or a rat anti-mouse GPVI (50 μg, mAb JAQ1, n=5) for another 4, 7, or 10 weeks twice a week by intraperitoneal injection. Finally, all mice were sacrificed at the age of 10, 13, or 16 weeks, respectively. The carotid arteries and aortic arch were processed for Sudan III staining and analyzed according to a standardized protocol as described²⁷.

### Deposit

A hybridoma cell line producing hGP 5C4 Fab antibody has been deposited under terms of the Budapest Treaty as hGP 5C4 with DSMZ-Deutsche Sammlung von Mikroorganismen und Zellkulturen on Nov. 25, 2003 and has been given Accession No. 2631.

### Example 9

GPVI mediates platelet adhesion to human atheromatous plaque and is critical for atheroprogression. Platelets play a critical role in development of atherosclerotic lesions, Ruggeri Z M. Nat. Med. November 2002; 8(11):1227-1234; Massberg S et al, J Exp Med. Oct. 7, 2002; 196(7):887-896; Theilmeier G et al, Blood. Jun. 15, 2002; 99(12):4486-4493; Huo Y et al, Nat. Med. January 2003; 9(1):61-67. During atherogenesis platelets adhere to the arterial wall and induce inflammatory and proliferative reactions resulting in lesion formation, (Massberg S et al, J Exp Med. Oct. 7, 2002; 196(7):887-896). Platelet adhesion induces platelet activation and secretion of potent proinflammatory compounds, (Gawaz M, Cardiovasc Res. Feb. 15, 2004; 61(3):498-511). Inhibition of platelet adhesion during early stage of atherosderosis reduces leucocyte accumulation in the arterial intima and attenuates atherosclerotic lesion formation, (Massberg S et al, J Exp Med. Oct. 7, 2002; 196(7):887-896).

Collagen is the main constituent of the plaque organ matrix and the predominant subtypes I and III represent 80-90% of total collagenous protein in atherosclerotic lesions, (Katsuda S, Kaji T. J Atheroscier Thromb. 2003; 10(5):267-274). While collagen type I heavily accumulates within the fibrous cap that overlies the lipid-rich core, collagen type III has been described to be predominant at the plaque/thrombus interface in regions of plaque erosion, (Fernandez-Ortiz A et al, J Am Coll Cardiol. June 1994; 23(7):1562-1569). Collagen acts as a strong activator of platelets and supports platelet adhesion both in vitro and in vivo. GPVI binds to fibrillar collagen, especially types I and III. Extracellular matrix collagen is exposed in microerosions of the vascular wall during atherogenesis resulting in enhanced transient platelet adhesion at lesions sites. Ligand binding to GPVI induces platelet activation leading to secretion and subsequent activation of integrin receptors that stabilize the platelet interaction with the arterial wall, (Cabeza N et al, Diabetes. August 2004; 53(8):2117-2121); (Nieswandt B, Watson S P, Blood. Jul. 15, 2003; 102(2):449-461).

Recently it was shown that collagen type I and type III containing structures in lipid rich atherosclerotic plaques stimulate thrombus formation by activating platelet GPVI, (Penz S et al, Faseb J. June 2005; 19(8):898-909). The present invention includes processes, products and uses relating to the molecular mechanisms of platelet adhesion to atherosclerotic plaques and investigates the effect of inhibition on atherosderotic lesion formation.

### Materials and Methods

### Reagents

The ant-GPVI mAb 5C4 and the irrelevant idiotype 2D1 were generated as described previously (Massberg S et al, Faseb J. February 2004; 18(2):397-399). MAb IB4 was raised in rats against human α2β1 integrin. Mouse-antibodies directed against human collagen type I and III, and IgG1-isotype control were purchased from Rockland Immunochemicals (Gilbertsville, Pa., USA). Soluble GPVI, the extracellular domain of human GPVI, was cloned and fused to the human Fc domain as described previously, (Massberg S et al, Faseb J. Feb. 2004; 18(2):397-399). For experimental procedures, control Fc and GPVI-Fc were purified as described, (Massberg S et al, Faseb J. February 2004; 18(2):397-399). Collagen (horn) was obtained from Nycomed Pharma (Munich, Germany). All other reagents used were from Sigma. Radio-labeled GPVI (¹²⁵IGPVI) was generated and performed as described, (Gawaz M et al, Thromb Haemost. May 2005; 93(5):910-913).

### Platelet Adhesion Under Low

Adhesion experiments were basically performed as previously described, (Massberg S et al, J Exp Med. Jan. 6, 2003; 197(1):41-49). Human whole blood from healthy volunteer donors, who had not taken any medication for at least 10 days before the experiment, was collected into syringes containing 0.5 vol % heparin. Platelets were labelled in whole blood by incubation with the fluorescent dye rhodamine-6G (final concentration 0.2 g/L) for 15 min at 37° C. Leukocytes could be readily distinguished from platelets by their larger size and nuclear morphology, red cells were not visualized by rhodamine-6G. Adhesion of platelets to coverslips coated with type I collagen, type III collagen, or human atheromatous plaque was determined in a parallel plate flow chamber in the presence of anti-GPVI (5C4, 10 μg/mL), anti-α2β1 integrin (IB4, 10 μg/mL), an irrelevant control antibody (2D1, 10 μg/mL), 0.8 mg/mL GPVI-Fc, or control Fc. In another set of experiments atheromatous plaque tissue was pre-incubated with function blocking antibodies recognizing human collagen type I or type III, or rabbit control IgG, coated onto the coverslips after 1 h incubation and perfused with whole blood as follows. Perfusion was performed for 10 minutes at a wall shear rate of 1000 s⁻¹, followed by a 4 min perfusion with Tyrode's solution-HEPES buffer at a shear rate of 1000/s using a pulse-free pump. The flow chamber was mounted on an inverted fluorescence microscope (Axiovert, Zeiss, Jena, Germany) and fluorescent images were recorded from microscope fields (20× objectives) using a digital photo camera (Axiocam, Zelss, Jena, Germany). All images were evaluated using a computer-assisted image analysis program (Cap Image 7.4). In experiments with collagen monolayers platelet surface coverage was assessed directly with the image analysis program. In experiments with atheromatous plaques, images in the phase-contrast (PC) and fluorescence microscopy mode were obtained, and recorded, before and after perfusion experiments. To quantify thrombus formation on plaque, the image taken from atheromatous plaque before perfusion was substracted offline from the fluorescent image recorded after 10 min perfusion using Adobe Photoshop (Adobe Systems, San Jose, Calif., USA). On the resulting images, platelet aggregates were calculated and related as percentage of plaque covered with platelet aggregates.

### Assessment of GPVI Binding to Immobilized Collagen and Atheromatous Plaque Material

Cell culture plates (Immulon2 HB, Dynex Technologies, Chantilly, Va.) were coated over night at 4° C. with 50 μl of diluted plaque or 1 μg collagen type I (bovine, BD Bioscience) or type III in 100 μl coating buffer (1.59 g/L Na₂ CO₃, 2.93 g/L NaHCO₃, 0.2 g/L NaH₃, pH 9.6). The plates were washed with 250 μl/well PBS/0.05% Tween 20 (PBST), blocked with Roti-Block (Roth, Karsruhe, Germany) over night and washed again twice with PBST. Then 20 μg/mL GPVI-Fc in PBST was added, incubated for 1 h at room temperature, and washed five times with 250 μl PBST. A peroxidase-conjugated goat anti-human IgG antibody Fcγ fragment specific (109-035-098, Dianova, Hamburg, Germany) was added in a dilution of 1:10.000 and incubated for 1 h at room temperature. After washing with PBST, 100 μl of detection reagent (BM Blue POD Substrate; Roche, Mannheim, Germany) was added and incubated for 10 min. The reaction was stopped by the addition of 100 μl 1 M H₂ SO₄, and the plate was measured at 450 nm against reference wavelength 690 nm.

### Platelet Aggregation

Platelet aggregation was performed in whole blood by luminometry, (Penz S et al, Faseb J. June 2005; 19(8):898-909). Collagen type I 1 μg/ml or collagen type III 1 μg/ml or extracted human atheromatous plaque tissue was added to citrated whole blood and stirred at 1000 rpm at 37° C. In some experiments aggregation was performed in the presence of anti-GPVI mAb 5C4 (5 μg/ml) or control IgG (5 μg/ml).

### Animals

Specific pathogen-free wild type C57BL6/J mice were obtained from Charles River (Suzfeld, Germany). 4-week-old male ApoE^{-/-} (C578L/63ApoEtm1Unc) mice (The Jackson Laboratory) consumed a 0.25% cholesterol diet (Harlan Research diets, 0% cholate) for 8 and 12 weeks as indicated.

### Detection of [¹²⁵]GPVI Binding In Vivo and Ex-Vivo

Wire-induced injury of the carotid artery in wild type and ApoE^{-/-} mice was performed as described¹². Approximately five minutes after induction of injury, a mixture of 1 MBq [¹²⁵I]GPVI and 7.4 MBq [¹²³I]GPVI in 100 μl buffer or equivalent amounts of the radioiodinated Fc-fragment were injected intravenously. Twenty-four hours p.i., planar whole-body images were acquired using a small animal positron emission tomography scanner (PET) (microPET Focus 120 scanner, CTI-Concorde Microsystems, Knoxville, Tenn., USA). Quantitative analysis was carried out by normalizing the regions of interest (ROI) at the lesion with the total activity concentration in the corresponding mouse. Values were expressed as % of the total activity found in the mouse. After imaging the animals were sacrificed. The injured and uninjured contralateral carotid arteries and the aortic arch were removed, counted in a gamma counter before rinsed with 1% paraformaldehyde, stained with Sudan III and evaluated by autoradiography. Therefore the arteries were placed on phosphor screens which were exposed for 4-7 days. For readout the Molecular Dynamics PhosphorImager 445 SI (Sunnyvale, USA) was used. Due to the low weight of the vessels measured activity was normalized to the vessel area determined via quantitative morphometry. Data are expressed as counts/mm².

### Chronic Administration of Soluble GPVI-Fc or Control Fc

4-week old ApoE mice were fed for further 8 or 12 weeks with 0.25% cholesterol diet (Harlan Research diets, 0% cholate). Animals were randomly assigned to receive either control Fc (n=4) 200 μg or GPVI-Fc (n=6) every other day intraperitoneal for the last 4 weeks. Thereafter animals were sacrificed at the age of 12 or 16 weeks respectively. Both carotid arteries and the aortic arch were excised and perfusion-fixed with 4% paraformaldehyde and stained with Sudan III to assess en face plaque extension as previously reported, (Massberg S et al, J Exp Med Oct. 7, 2002; 196(7):887-896).

### Preparation of Human Carotid Atheromatous Plaque and Immunohistochemistry

Atherosclerotic tissue specimens were obtained from patients who underwent surgery for high grade carotid artery stenosis as described previously, (Penz S et al, Faseb J. June 2005; 19(8):898-909). In brief, the carotid plaque tissue was removed by a technique of intraoperative endarterectomy that preserved the plaque structure en bloc. The specimens containing lipid-rich soft plaques were collected. The atheromatous plaques were carefully dissected from other regions of the atherosclerotic tissue specimens, weighed, homogenized and stored at −20° C. The atheromatous plaque concentration was 50 mg/mL wet weight corresponding to protein concentrations of 0.5-2.5 mg/mL. For flow chamber experiments, homogenized atheromatous plaque (5 mg/mL) was dissolved in PBS containing 15 mM fatty acid-free albumin, incubated for 16 h at 4° C. in PBS-albumin (75 mM) to prevent non-specific binding, and immobilized onto glass coverslips. Where indicated, atheromatous plaque was pre-incubated for 1 h with anti-human collagen type I or type III antibodies or rabbit IgG control and thereafter coated onto coverslips as described. For immunohistochemical localization of collagen type I and III structures in situ, atheromatous plaques were fixed in 4% paraformaldehyde (pH 7.0) and embedded in paraffin as described. Serial paraffin sections of 3 μm thickness were deparaffinized and dehydrated. The specimens were microwaved in citrate buffer (pH 6.0) for 20 min. Endogenous peroxidase was blocked by 3% H₂O₂, followed by incubation with anti-collagen type I or type III antibodies, or IgG (control) antibody. Collagen-positive structures were identified by using the streptavidin-horseradish peroxidase technique (DakoChem Mate Detection kit; Hamburg, Germany). Specific binding of the antibodies was detected by 0.1% 3′3′-diaminobenzidine. For immunohistochemical localization of GPVI-binding sites in situ, atheromatous plaques were processed as described above and stained with GPVI-Fc.

### Statistical Analysis

Data represent mean±s.e.m. Data were analyzed using one-way ANOVA with a Tukey post-hoc test. A value of P<0.05 was regarded as significant.

### Results

Recently, collagen type I- and type III-containing structures and the platelet collagen receptor GPVI has been shown to be essential for atherosclerotic plaque-induced thrombus formation, (Penz S et al, Faseb J. June 2005; 19(8):898-909; Cosemans J M et al, Atheroscierosis. July 2005; 181(1):19-27). To explore whether atheromatous plaque contains GPVI binding activity in situ, immunostaining of human carotid atheromatous plaque sections with anti-collagen type I and type III, or human soluble Fc-GPVI was performed (n=8) (data not shown). The presence of collagen type I- and type III-containing structures both in the cap as well as in the core region of plaques in situ was revealed (data not shown). The pattern of GPVI-Fc binding was similar to the immunostaining pattern of collagen type III and differed substantially from the distribution pattern of collagen type I structures (data not shown). Maximal in situ binding of soluble GPVI was found in the core region (data not shown).

Next, binding of soluble GPVI-Fc to atheromatous plaque (AP) material isolated from the core region of human carotid plaque specimen was quantified. Substantial and specific binding activity of GPVI-Fc to atheromatous plaque material was found (data not shown), indicating that GPVI interacts with collagen-containing structures present within the atherosclerotic plaque tissue. Binding of soluble GPVI-Fc was enhanced to immobilized collagen type III compared with collagen type I (data not shown). Thus, it is shown that soluble GPVI preferentially binds to collagen structure within the core region of human atherosclerotic plaque.

Recently, it was shown that GPVI is essential for plaque-induced thrombus formation, (Penz S et al, Faseb J. June 2005; 19(8):898-909; Cosemans J M et al, Atherosclerosis. July 2005; 181(1):19-27). To further analyze the role of GPVI for plaque-induced platelet aggregation isolated atherosclerotic plaque tissue was added to whole blood and platelet aggregation was monitored by impedance measurement, (Penz S et al, Faseb J. June 2005; 19(8):898-909). Extracted AP material was found to induce substantial platelet aggregation similar to collagen (data not shown). ATP-Induced platelet aggregation was substantially reduced in the presence of a blocking anti-GPVI mAb (5C4) but not in the presence of control mAb (data not shown).

Platelet adhesion to collagen is mediated both through GPVI and the integrin collagen receptor α₂β₁, (Nieswandt B, Watson S P, Blood. Jul. 15, 2003; 102(2):449-461). To evaluate the relative importance of these two collagen receptors for platelet adhesion to AP material, platelet adhesion under dynamic flow conditions was studied using cover slips coated with extracted AP material or collagen type I and III. Significant platelet adhesion to immobilized collagen type I and type III was found. Platelet adhesion to both collagen type I and III was significantly reduced by inhibition of GPVI with the neutralizing anti-GPVI Ab 5C4 (data not shown). In contrast a blocking anti-α_{z}β₁ (IB4) mAb inhibited platelet adhesion to collagen platelet type I but to a much lesser extent to collagen type III (data now shown). Further, inhibition of platelet adhesion through soluble GPVI-Fc was found to be maximal to collagen type III and moderate to type I.

To explore the importance of GPVI and α₂β₁ for platelet adhesion onto immobilized AP material, cover slips that were coated with AP extracts were perfused with whole blood in the absence and presence of mAb 5C4 (anti-GPVI) and IB4 (antiα₂β₁). It was found that anti-GPVI mAb but not antiα₂β₁ substantially reduced platelet adhesion to immobilized AP material under flow conditions (data not shown). Further, pre-incubation of AP with soluble GPVI—Fc but not with Fc control resulted in inhibition of platelet adhesion to immobilized AP (data not shown). This indicates that GPVI is the critical collagen receptor for platelet interaction with AP. In control experiments, immobilized AP material was prej-incubated with anti-Col I and anti-Col I mAb, or both, and significant reduction of platelet adhesion was found (data not shown). Thus, GPVI is a critical receptor for platelet interaction with human atheromatous plaque material.

To test whether platelet GPVI binds to atherosclerotic lesions in vivo, soluble Fc-GPVI-Fc or control Fc fragment was radio-labelled with iodine (¹²⁵I) and injected into ApoE-deficient or wild type mice, (Gawaz M et al, Thromb Haemost. May 2005; 93(5):910-913). Ex vivo autoradiography of the explanted aortic arch revealed a substantial approximately 1.5-fold increase in ¹²⁵I-GPVI binding activity of ApoE-deficient mice compared with wild type after tracer administration (ApoE vs wild type, counts/mm²: 356±45 vs 250±10) (p<0.01) (data not shown). Similarly, in vivo positron emission tomography (PET) imaging revealed substantial accumulation of radioiodinated GPVI-Fc in the aortic arch of ApoE-deficient mice but not wild type mice (data not shown). As control, radioiodinated GPVI-Fc accumulated to a similar degree in wire-induced lesions of the right carotid artery (data not shown). Thus, GPVI binds to atherosclerotic lesions of ApoE-deficient mice in vivo.

Recently, it was demonstrated that platelet adhesion to endothelium is critical for atherogenesis in mice, (Massberg S et al, J Exp Med. Oct. 7, 2002; 196(7):887-896; Massberg S et al, Circulation. Aug. 23, 2005; 112(8):1180-1188). To determine the role of GPVI-mediated platelet adhesion to the arterial wall for vascular remodelling in atherogenic mice, 8- and 12-weeks old ApoE^{-/-} mice with soluble GPVI-Fc or control Fc were treated over a period of 4 weeks by intraperitoneal injection of 200 μg every other day. Thereafter, the degree of lesion formation was assessed in the aortic arch thereafter at the age of 12 and 16 weeks, respectively. Prolonged administration of GPVI-Fc (n=x) but not of Fc (n=x) significantly limited lesion formation in 16-weeks old mice. Quantitative computer-assisted image analysis of the aortic arch showed an approximately 35% reduction (p<0.01) in the extent of fatty streak formation in 16-week-old ApoE^{-/-} mice, as assessed by Sudan-III staining (data not shown). Thus, prolonged administration of soluble GPVI attenuates atherosclerotic lesion formation in ApoE-deficient mice.

The present disclosure therefore relates to the following: 1) the platelet collagen GPVI binds to atheromatous plaque tissue in situ and recognizes preferentially collagen structures within the core region of plaque tissue. 2) GPVI but not integrin α₂β₁ is the predominant platelet collagen receptor that mediates adhesion of platelets to atheromatous plaque material under flow conditions. 3) The soluble GPVI receptor (for example in the form of a GPVI fusion protein) substantially binds to atherosderotic arteries of ApoE-deficient mice in vivo. 4) Prolonged administration of soluble GPVI but not control Fc attenuated atheroprogression in ApoE-deficient mice. The findings indicate that the GPVI is critical for platelet adhesion to atheromatous arteries and plays a critical role in atheroprogression.

### Example 10

Biodistribution of GPVI-Fc fusion protein having the sequence shown in FIG. 7 (PR-15) was assessed by using ¹²⁵I-radiolabelled GPVI-Fc in mice. In initial experiments, it was shown that collagen binding capacity and affinity of GPVI-Fc were not affected by radiolabelling. Radioactivity was counted 30 min, 2 hours, 6 hours and 24 hours after intravenous application in the respective organs and in the serum. Uptake of free atomic iodine to the thyroid gland was blocked by administration of perchlorate to the animals prior to the experiment. Radioactivity was determined by gamma-counting in the serum, the blood, the liver, the kidney, the spleen, the heart, the lung, the intestines, in skeletal muscle and the brain on a counts per tissue weight/tissue volume basis. For this purpose, the respective animals were sacrificed after the indicated time periods after administration of GPVI-Fc, and organs were harvested by necropsy.

Radioactivity in the blood and in the serum reached a maximum 30 minutes after intravenous administration and declined progressively over the following 24 hours. Half-maximal values were observed after 4-5 hours. The differences in plasma half-lives as determined by ELISA detection of GPVI-Fc and those determined by radioactivity counting are probably due to degradation of some parts of the protein into polypeptides occurring in circulating blood, starting 60 minutes after the injection.

Initial GPVI-Fc concentrations per tissue volume (30 minutes after intravenous administration) clearly depended on the relative perfusion of the respective organs: Compared to circulating blood, it was almost equal in the liver, and clearly lower in the kidney, the heart, the spleen, the intestines, and in the skin. Hardly any radioactivity was measured in the brain and in skeletal muscle, indicating that the blood brain barrier is not crossed by GPVI-Fc (PR-15).

Over the following hours, radioactivity declined also in the liver, the heart, the spleen, the intestines and in the brain in a comparable manner to that in the blood and serum. Consequently, these studies did not reveal any delay in clearance from any of these organs. In contrast, unchanged levels of radioactivity were determined in the kidney, corresponding to the formation of primary urine in that organ and successive renal excretion of GPVI-Fc. Intrarenal complex formation could be excluded. Radioactivity uptake in the thyroid gland was increased, but not different to that observed after administration of other ¹²⁵I-labelled peptides, so that it must be ascribed to non-specific free iodine uptake.

Binding of radiolabelled GPVI-Fc to arterial lesions was tested in Apo E^{-/-} mice in vivo, and compared to healthy wild type mice. The aortae and the carotid arteries were dissected from mice 24 hours after intravenous administration of GPVI-Fc. The atherosclerotic arteries were exposed to phospho imagers to detect specific GPVI-Fc signals. GPVI-Fc specifically labelled arterial lesions, as determined by subsequent sudan oil red staining of the arteries.

### Example 11

In a similar approach (to that of Example 10), specific binding of radiolabeled GPVI-Fc (PR-15) to lesions in the vessel wall was also detected after mechanical injury of the carotid artery in vivo. The carotid arteries were investigated 24 hours after intravenous administration of GPVI-Fc.

In order to countercheck the specificity of the approach, a set of control experiments investigated the binding of equal doses of control Fc protein, which had been radiolabelled in the same way. In these experiments, no specific binding of Fc to arterial lesions was documented, thus underlining the specificity of GPVI-Fc.

In vivo positron emission tomography imaging allowed to detect this binding in vivo. Radioactive signals were enhanced at a mechanical injury site. In contrast, no specific signal occurred in uninjured, healthy mice. Similar results were obtained 8, 24 and 48 hours after intravenous administration of GPVI-Fc in these mice. Also from these experiments, it can be assumed that the biological half-life of GPVI-Fc is at least 24 hours.

### Example 12

Local Delivery of Soluble Platelet Receptor GPVI Inhibits Thrombin Formation In Vivo

The present invention includes the feasibility methods including local administration and delivery of anti-GPVI agents. Thus, the following examples demonstrate an effect of local delivery of, for example, soluble GPVI on thrombus formation at the site of balloon-induced carotid injury in rabbits.

### Methods

### Animals

New Zealand White rabbits (weight 3.6±0.3 Kg; from Asam, Aretsried, Germany) were kept under constant temperature and humidity with chow ad libidum according to the animal health regulations. The animals are anaesthesised with Propofol (Propofol, 2%, Fresenius) and Fentanyl (Fentanyl-Janssen 0.5 mg, Janssen-Cilag, 0.01 mg/kg). They are intubated and artificially ventilated (AWS, Fa. Völker, Germany). For analgesia all animals obtained Buprenorphin (Terngesic®, Boehringer, 0.01 mg/kg) and before the intervention anticoagulation with 100 IU/kg Heparin was induced.

### Microsurgical Procedure and Balloon Injury Model Endothelial Denudation in the Right Common Carotid Artery:

Left femoral artery was prepared and an introducer for arterial catheterisation was placed (Radiofocus®, 4F, Terumo). Under x-ray guidance a Fogarty catheter (EMB: thru-lumen embolectomy catheter, 3F, Edwards Lifesciences, Guidewire: Stabilizer®, 014 in., Cordis) is placed in the right carotid artery. After inflation with 0.4 ml air the balloon is gently rubbed up and down twice between the second and the sixth cervical vertebrae.

### Local Drug Delivery

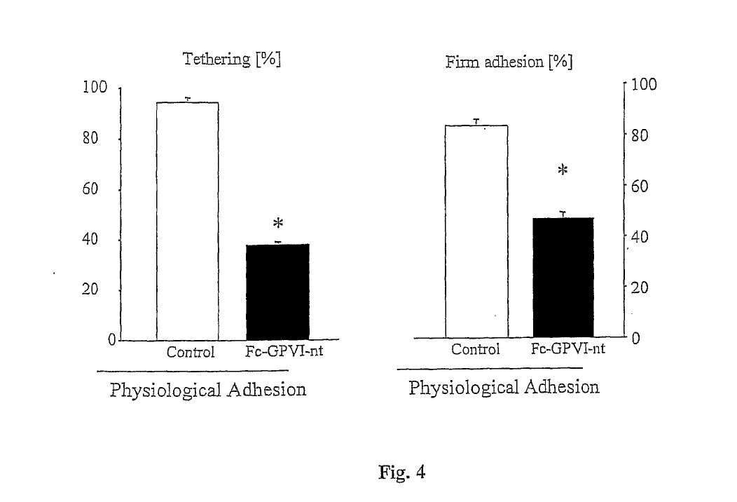

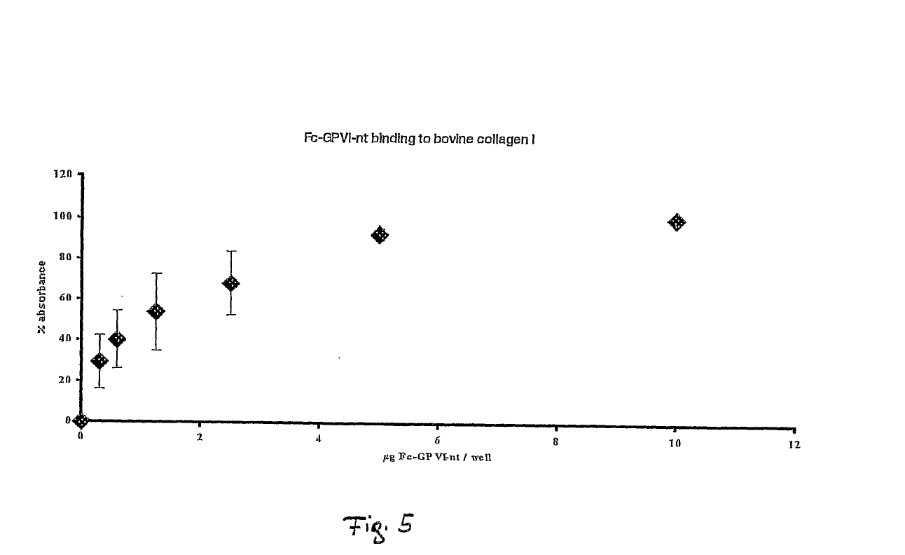

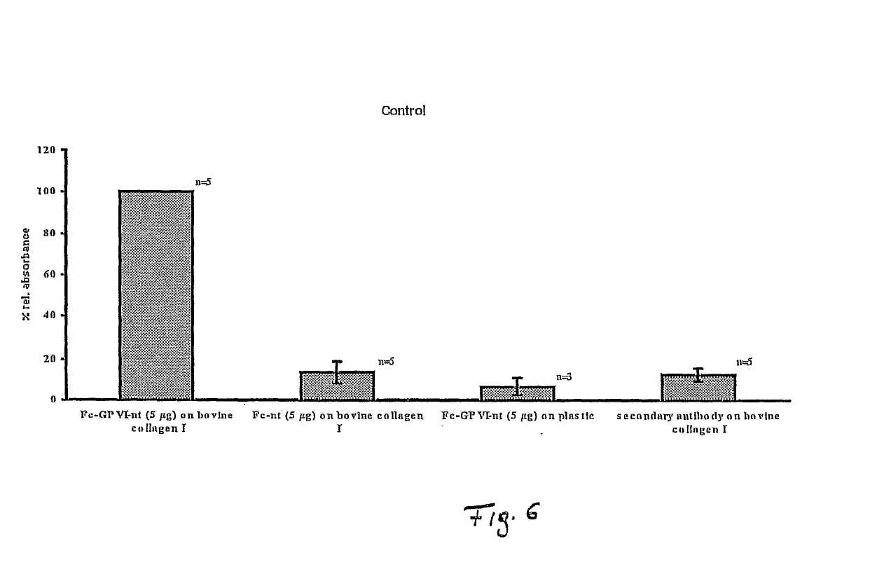

A 3.0 mm modified double-balloon catheter (ACROSTAK Corp., Winterthur, Switzerland) was used for local GPVI (In the form of GPVI-Fc fusion protein) delivery. This system consists essentially of a percutaneous balloon having a distal and proximal segment with occlusive function and a central segment that allows for homogenous transfer of GPVI to the vessel wall. Three holes in the distal ramp fill the drug depot without additional trauma or hydrojets (see FIG. 4). The balloon was inflated at a low pressure of 2 atm that allows for distal and proximal occlusion of the vessel without additional trauma while simultaneously forming a central drug depot. Local drug delivery of FC-GPVI or FC solution (1.3 mg/ml) is carried out under continuous infusion of 3 ml for 15 minutes. After the drug delivery the catheters are removed the femoral artery is ligated and the inguinal wound is sutured by using Vicryl® (3-0).

### Immunohistological Analysis

Four hours after the drug delivery the animals are euthanised. After arterial fixation with formaldehyde (2%, 500 ml for 10 minutes) in vivo the vessels are removed, embedded in OCT compound (Leica, Germany) and frozen in liquid nitrogen. Serial 10-μm-thick cryosections with 2 mm intervals were cut and finally post-fixed with acetone, Detection of GPVI-Fc binding was done with an antibody raised against GPVI (anti GPVI antibody 4C9) at a dilution of 1:100. Antibody binding was visualised with an avidin/biotinylated glucose oxidase (ABC-GO) system according to the manufacturer's instructions (Vectastaln ABC-GO Kit; Vector Laboratories Inc; Burlingame; CA 94010).

### Quantification of Intravascular Thrombus Formation

After fixation, macroscopical En Face preparation of the whole carotid artery was performed as previously described. The whole length of the left and right common carotid artery (5.2 cm) were removed after perfusion fixation. The arteries were cleaned of adventitia and connective tissue and segments were cut open longitudinally with the endothelium facing up. After mounting of vessels on glass slides, vessels were viewed macroscopically. The thrombus area was quantified using a computer-assisted image analysis software (Scion image) and the thrombus area was calculated relative to the endothelium surface. (Torsney E et al, Circ Res 2004; 94: 1466-1473).

### Statistical Analysis

Results in this study were expressed as mean±SD. Statistical analysis was performed with Student t test or ANOVA where appropriate. A value of P<0.05 was considered statistically significant.

### Results

Systemic application of a dimeric form of soluble GPVI (sGPVI) has been shown to inhibit thrombus formation at site of arterial injury in mice (Massberg et al, Faseb J. February 2004; 18(2):397-399). The present examples addressed whether local delivery of sGPVI can reduce thrombus formation at site of arterial injury. After endothelial denudation and balloon-injury of the right carotid artery of New Zealand rabbits, reproducible thrombus formation at the site of injury occurred (FIG. 22).

To assess the efficacy of local balloon-based delivery of sGPVI, immunostaining of serial cross sections of carotid arteries were performed. Carotid cross sections were stained with a specific monoclonal antibody 5C4 directed against GPVI. Substantial positive staining for GPVI of injured carotid segments, but not of intact carotid arteries (FIG. 23).

The present invention also addresses whether a local delivery of sGPVI attenuates thrombus formation at site of carotid injury. Using a modified double-balloon drug-delivery catheter GPVI (10 μg/ml) or control protein (Fc (10 μg/ml)) was delivered to site of injury, and the respective carotid segments were incubated for 5 min with sGPVI or the control. Thereafter, thrombus formation was assessed by direct microscopy (FIG. 24). It was found that sGPVI (Pr-15) treatment of carotid lesions resulted in an approximately 80% reduction of thrombus formation (10.xx vs. 2.yy, p<0.0x) (FIG. 24). Thus, local delivery of sGPVI substantially reduces thrombus formation at site of vascular lesions in this animal model. The present disclosure shows that local delivery of the soluble form of platelet collagen receptor glycoprotein VI (sGPVI) efficiently reduces thrombus formation at sites of arterial injury. Thus, the present disclosure includes a new antithrombotic approach to treat spontaneous or ballon-induced arterial lesions without the necessity of systemic antiplatelet therapy. Thrombotic complications arise from arterial segments that are treated with percutaneous coronary interventions (PCI). The introduction of intensified combined antiplatelet therapy has significantly lowered the rate of thrombo-ischemic complications significantly after PCI, however, subacute thrombotic vessel occlusion still occurs in 1 to 4% of patients after coronary stenting.

In the present disclosure, the effect of soluble GPVI (in the form of PR-15) on thrombus formation at site of ballon-injury after local delivery of this soluble receptor using a new drug delivery balloon was evaluated. It was shown that, using this drug-delivery ballon, soluble GPVI can be sufficiently delivered locally at site of vascular injury. Further, localized treatment of injured arterial arteries with sGPVI efficiently reduces thrombus formation. Thus, the present disclosure includes a method of localized antithrombotic treatment of thrombogenic vulnerable atherosclerotic plaques or stented artery segments without systemic antiplatelet effects.

### Example 13

FIGS. 25 to 27 show a comparison between a GPVI-Fc fusion protein (PR-15), whose sequence is shown in FIG. 7 and a GPVI-Fc fusion protein comprising an Ala-Ala-Ala linker linking the extracellular domain of GPVI and the Fc portion.

### 1. Methods

### Cloning

Fc-GPVI (PR-15) and Fc (a control protein) were generated and purified as described in PCT/EP03/05929. A competing Fc-GPVI-nt with another specific linker according to the sequence alluded to in WO 01/00810 was cloned according to the same method. For this purpose, a similar construct was generated using the same DNA sequence as descried in FIG. 7 of PCT/EP03/05929 except for the Gly-Gly-Arg linker on amino acid positions 270-272, which was replaced by a Ala-Ala-Ala linker, amplified and purified according to the same methods.

### Aggregation and ATP Release

Measurements of aggregation and ATP release were carried out with a standardized device as described in PCT/EP03/05929.

### Thrombus Formation In Vivo

### Preparation and Measurement of Thrombus Formation in the Carotid Artery

Vascular injury was induced in the mouse common carotid artery. C57BL6/J mice were anesthetized by intraperitoneal injection of a solution of xylazine (5 mg/kg body weight, AnaSed; Lloyd Laboratories) and ketamine (80 mg/kg body weight, Ketaset; Aveco Co., Inc.). A catheter was introduced into the tall vein, and either Fc-GPVI-nt or control Fc were injected at doses of 2 mg/kg body weight, and flushed with 200 μL saline. Then, the right cervical region was dissected free between the sternohyoid and myohyoid muscle, a clamping and subsequent ligation of the cranial internal carotid artery was carried out. Then, the common carotid and the external carotid were clamped. The right internal carotid artery was incised. A think coronary guiding wire of defined size was introduced into the right common carotid artery and a vascular injury was induced by moving the wire up and down three times. This led to complete loss of endothelial cells at the site of injury.

Thereafter, the animals were allowed to sit up and survived for four hours after the intervention. Then, anaesthesia was reinduced, and the animals were perfused with 10 mL of saline solution through their tail veins, followed by a second infusion of 4 mL paraformaline (PFA, 4%). Then, the large vessels including both carotid arteries (Including the bifurcation and the common carotids up to 3.5 mm proximal to the carotid bifurcation) were dissected free and taken out. Fat tissue was removed. The vessels were then opened longitudinally and spread out on microscopic cover slips.

The en face thrombus size was measured by digitalized imaging analysis, which measured the relative thrombus area as a percentage of the total vessel area as spread out on the slide.

### Aggregation and ATP Release

FIG. 25 shows the results of aggregation studies. Aggregation and ATP release measurements of freshly isolated human platelets in response to collagen (1 μg/mL) resulted in strong photometric signals as described in PCT/EP03/05929).

FIG. 25 shows the aggregation as a percentage of the internal standard of the aggregometric device. The aggregation was slightly but not significantly less in the presence of a control Fc protein as compared to collagen-Induced aggregation. Aggregation was inhibited slightly by the GPVI fusion protein containing the triple alanine linker. In contrast, administration of the GPVI fusion protein containing the Gly-Gly-Arg linker (PR-15) at the same concentration (40 μg/mL) markedly and highly significantly suppressed aggregation. Shown are results from 4 independent experiments. *p<0.05 vs controls.

It can be seen from FIG. 25 that administration of collagen only induced aggregation of approximately 37 to 38% of the internal standard of the device used. Administration of collagen together with a control Fc portion induced aggregation of approximately 26% of the internal standard of the device, whilst administration of collagen and a GPVI-Fc fusion protein containing a triple alanine linker resulted in approximately 20% aggregation of platelets when compared to the internal standard of the device used. In contrast, the administration of GPVI-Fc fusion protein comprising a Gly-Gly-Arg linker with collagen induced only approximately 4% of aggregation as compared to the internal standard of the device used.

It will be apparent therefore that the GPVI-Fc fusion protein containing a Gly-Gly-Arg linker is capable of inhibiting platelet aggregation in response to collagen by at least 50%. Particularly, the GPVI-Fc fusion protein containing a Gly-Gly-Arg linker is capable of inhibiting platelet aggregation in response to collagen by at least 60%, for example at least 70%, e.g. 75% or more.

### Endothelial Lesion In Vivo and Determination of En Face Thrombus Size

FIG. 26 shows the results of mean ATP release from platelets in response to 1 μg/mL of collagen in the presence of either a GPVI-Fc fusion protein containing a triple alanine or a GPVI-Fc fusion protein containing a Gly-Gly-Arg linker.

FIG. 26 shows mean ATP release from human platelets in response to 1 μg/mL collagen (expressed as % of the internal standard) in the presence of either construct. ATP release was slightly but not significantly reduced in the presence of the control Fc, and slightly more reduced in the presence of the Fc-GPVI-nt containing the Ala-Ala-Ala linker. In contrast, administration of the Fc-GPVI-nt containing the Gly-Gly-Arg linker at the same concentration (40 μg/mL) markedly and highly significantly suppressed ATP release. Shown are results from four independent experiments. *<0.05 vs. controls.

It is apparent from FIG. 26 that the administration of collagen causes 100% of ATP release from platelets. Administration of collagen together with a Fc portion causes approximately 55% of ATP release. There is 40% of ATP released from platelets when collagen and the GPVI-Fc fusion protein containing triple alanine linker were administered. Administration of collagen and the GPVI-Fc fusion protein containing the Gly-Gly-Arg resulted in only approximately 10% of ATP release. That is to say, the GPVI-Fc fusion protein containing the Gly-Gly-Arg linker inhibited approximately 90% of collagen-Induced ATP release. The fusion protein containing the triple alanine linker inhibited only 60% of collagen-ATP release from platelets. It has therefore been demonstrated that the GPVI-Gly-Gly-Arg-Fc fusion protein is capable of inhibiting collagen-Induced ATP release from platelets by at least 65%, optionally by at least 70%, optionally by at least 80% and optionally by at least 90%.

### Adhesion to Endothelial Lesion In Vivo

FIG. 27 shows the adhesion of platelets to endothelial lesions in vivo. A comparison between an Fc portion, the GPVI-Fc fusion protein containing a triple alanine linker and a GPVI-Fc fusion protein containing a Gly-Gly-Arg linker (PR-15) was made.

Mice were injected with 2 mg/kg of either construct, then endothelial lesions were induced. The right carotid arteries were excised, and prepared as described in Methods. The en face thrombus size was related to the overall surface of the spread-out vessel and expressed as a percentage. FIG. 27 shows the results. It is clear that administration of the Fc-GPVI-nt construct (PR-15) containing the specific Gly-Gly-Arg linker markedly and significantly inhibits thrombus formation at the endothelial lesion of the right carotid artery, where the lesion had been placed. In contrast, the administration of the Fc-GPVI-nt construct containing the specific Ala-Ala-Ala linker resulted in only a trend towards decreased thrombus formation which did not reach statistical significance.

The uninjured left carotid arteries were investigated as controls in all animals and did not show any platelet adhesion or thrombus formation.

Mice were injected with 2 mg/kg of either construct, then endothelial lesions were induced. The right carotid arteries were excised, and prepared as described above. The Figure shows the mean en face thrombus sizes related to the overall surface of the respective vessel and expressed as a percentage of the total vessel area as spread out on the slide. Shown are results form five independent experiments in five independent animals.

FIG. 27 shows that the administration of an Fc portion to the mice resulted in formation of thrombus of approximately 7.5% in area relative to the total vessel area as spread out on a slide. Administration of the GPVI-Fc fusion protein containing the triple alanine linker resulted in the formation of a thrombus of approximately 4% in area relative to the total vessel area as spread out on a slide. Administration of the GPVI-Fc fusion protein containing the Gly-Gly-Arg linker resulted in the formation of a thrombus of negligible area relative to the total vessel area as spread out on a slide. It is therefore considered that the GPVI-Fc fusion protein containing the Gly-Gly-Arg linker is capable of inhibiting thrombus formation caused by induction of arterial lesions in a mouse by at least 70% as compared to a control administered with an Fc portion polypeptide. Indeed, the GPVI-Fc fusion protein containing the Gly-Gly-Arg linker is capable of inhibiting the thrombus formation by at least 80% and optionally at least 90% and optionally at least 95%.

The disclosure further includes the subject matter of the following paragraphs:

1. An agent which is capable of binding to a plurality of GPVI-binding sites, optionally a plurality of types of GPVI-binding sites, of a dysfunctional, inflamed or atherosclerotic blood vessel area, the agent optionally being capable of inhibiting collagen-induced ATP release from human platelets by at least 65%.

2. The agent of paragraph 1 which is capable of inhibiting ATP release from human platelets by at least 70%.

3. The agent of paragraph 1 which is capable of inhibiting ATP release from human platelets by at least 80%, e.g. at least 90%.

4. An agent which is capable of inhibiting GPVI interaction with collagen and a molecule selected from the group consisting of vitronectin, fibronectin and combinations thereof, the agent being capable of inhibiting collagen-induced ATP release from human platelets by at least 65%, e.g. at least 70%, for example 80% or more as in the case of inhibition of at least 90%.

5. An agent which is capable of binding to a plurality of GPVI-binding sites, optionally a plurality of types of GPVI-binding sites, of a dysfunctional, inflamed or atherosclerotic blood vessel area, the agent being capable of inhibiting thrombus formation caused by induction of arterial lesions in the mouse by at least 70% as compared with a control administered with an Fc polypeptide, the relative inhibitions of thrombus formation being determined by measurement of the en face thrombus size as a percentage of an artery wall area placed on a surface for the purpose of said measurement.

6. The agent of paragraph 5 which inhibits the thrombus formation by at least 80%.

7. The agent of paragraph 5 which inhibits the thrombus formation by at least 90%, e.g. at least 95%.

8. An agent which is capable of inhibiting GPVI interaction with collagen and a molecule selected from the group consisting of vitronectin, fibronectin and combinations thereof, the agent being capable of inhibiting thrombus formation caused by induction of arterial lesions in the mouse by at least 70%, e.g. at least 80% and for example at least 90%, e.g. at least 95%, as compared with a control not administered with an Fc polypeptide, the relative inhibitions of thrombus formation being determined by measurement of the en face thrombus size as a percentage of an artery wall area placed on a surface for the purpose of said measurement.

9. An agent which is capable of binding to a plurality of GPVI-binding sites, optionally a plurality of types of GPVI-binding sites, of a dysfunctional, inflamed or atherosclerotic blood vessel area, the agent being capable of inhibiting platelet aggregation in response to collagen by at least 50%.

10. The agent of paragraph 9 which inhibits such platelet aggregation by at least 60%, for example at least 70%, e.g. 75% or more.

11. An agent which is capable of inhibiting binding of GPVI to collagen and a molecule selected from the group consisting of vitronectin, fibronectin and combinations thereof, the agent being capable of inhibiting platelet aggregation in response to collagen by at least 50%, e.g. at least 60%, for example at least 70%, e.g. 75% or more.

12. The agent of any of paragraphs 1 to 3, 5 to 7, 9 and 10 which is capable of inhibiting the binding of GPVI to collagen and a protein selected from fibronectin, vitronectin and combinations thereof.

13. The agent of any of paragraphs 1 to 3, 5 to 7, 9, 10 and 12 wherein the blood vessel area is a human blood vessel area.

14. The agent of any of paragraphs 4, 8 and 11, and paragraph 13 in combination with paragraph 12, in which the collagen, fibronectin and vitronectin are human.

15. The agent of any preceding paragraph which is not a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

16. The agent of any preceding paragraph which comprises an amino acid sequence of or comprised in an extracellular domain of GPVI, or a variant thereof that is functional for binding to collagen and to at least one protein selected from fibronectin and vitronectin.

17. The agent of any preceding paragraph, which comprises an amino acid sequence encoded by:
(i) a nucleic acid sequence which encodes a wild type GPVI extracellular domain;
(ii) a nucleic acid sequence which hybridises to said nucleic acid sequence (i) and which encodes a polypeptide that is capable of binding to a plurality of types of GPVI-binding sites of a dysfunctional, inflamed or atherosclerotic blood vessel area; or
(iii) a nucleic acid sequence which differs from said nucleic acid sequence (I) by virtue of the degeneracy of the genetic code.

18. The agent of any preceding paragraph, which comprises an amino acid sequence encoded by:
(i) a nucleic acid sequence which encodes a wild type GPVI extracellular domain;
(ii) a nucleic acid sequence which hybridises to said nucleic acid sequence (i) and which encodes a polypeptide that is which is capable of inhibiting the binding of GPVI to collagen and to one or both of fibronectin and vitronectin; or
(iii) a nucleic acid sequence which differs from said nucleic acid sequence (i) by virtue of the degeneracy of the genetic code.

19. The agent of any preceding paragraph, which comprises an amino acid sequence encoded by:
(i) a nucleic acid sequence of bases 1 to 807 of SEQ ID No. 2 (FIG. 8);
(ii) a nucleic acid sequence which hybridises to bases 1 to 807 of SEQ ID No. 2 (FIG. 8) and which encodes a polypeptide that is capable of binding to a plurality of types of GPVI-binding sites of a dysfunctional or atherosclerotic blood vessel area; or
(iii) a nucleic acid sequence which differs from bases 1 to 807 of SEQ ID No. 2 (FIG. 8) by virtue of the degeneracy of the genetic code.

20. The agent of any preceding paragraph, which comprises an amino acid sequence encoded by:
(i) a nucleic acid sequence of bases 1 to 807 of SEQ ID No. 2 (FIG. 8);
(ii) a nucleic add sequence which hybridises to bases 1 to 807 of SEQ ID No. 2 (FIG. 8) and which encodes a polypeptide that is which is capable of inhibiting the binding of GPVI to collagen and to one or both of fibronectin and vitronectin; or
(iii) a nucleic acid sequence which differs from bases 1 to 807 of SEQ ID No. 2 (FIG. 8) by virtue of the degeneracy of the genetic code.

21. The agent of any of paragraphs 16 to 20 wherein said amino acid sequence is linked to an antibody-derived amino acid sequence.

22. The agent of paragraph 21 wherein said amino acid sequence is linked at its C-terminus to the N-terminus of the antibody-derived sequence through a linker comprising a hydrophilic amino acid.

23. The agent of paragraph 21 wherein the antibody-derived amino acid sequence is an Fc domain of an immunoglobulin.

24. The agent of any of paragraphs 16 to 23 which comprises a plasma-soluble protein.

25. The agent of any of paragraphs 5 to 10, permitted combinations of paragraphs 5 to 10 and subsequent paragraphs and paragraph 24 wherein Fc is a human IgG Fc.

26. The agent of any of paragraphs 1 to 15 which is as defined in any of paragraphs A1 to A29, A33 to A64 and A68 to A105.

27. The agent of any of paragraphs 1 to 15 which is not as defined in any of paragraphs A1 to A29, A33 to A64 and A68 to A105.

28. The agent of any of paragraphs 1 to 14 and 27 which is not an agent as defined in any one or more of paragraphs 16, 17, 18, 19, 20, 21, 22, 23, 24 and 25.

29. The agent of any of paragraphs 1 to 14 which comprises an antibody product selected from antibodies and antibody fragments; a protein; a polypeptide; a fusion protein; an aptamer; or a compound.

30. The agent of any of paragraphs 1 to 16, which comprises an antibody product which comprises domains which recognize collagen, particularly collagen I and/or collagen III, fibronectin and vitronectin.

31. The agent of paragraph 29 or paragraph 30 wherein the antibody product is a monoclonal antibody, a polyclonal antibody, a chimeric antibody, a human antibody or a humanized antibody or a fragment of such an antibody.

32. The agent of paragraph 29, paragraph 30 or paragraph 31 wherein the antibody fragment is a Fab fragment, a F(ab)₂ fragment, a scFv, a Fv fragment or a single domain antibody.

33. The agent of any of paragraphs 1 to 14 which comprises a protein or polypeptide derived from a snake venom, e.g. a protein derived from Crovidisin, a collagen-binding protein isolated from snake venom of Crotalus viridis.

34. The agent of paragraph 33 wherein the protein or polypeptide is a recombinant protein or polypeptide which comprises domains which bind to collagen, fibronectin and vitronectin.

35. An agent which comprises:
a first portion capable of binding to an intravascular GPVI-binding domain; and
a second portion which is capable of binding to a site other than a GPVI-binding domain and/or comprises a therapeutic or diagnostic moiety which has a direct or indirect therapeutic or diagnostic function
   or a combination of such agents.

36. An agent of paragraph 35 wherein the second portion is capable of binding to a binding site for a platelet-bound ligand other than GPVI, for example GPIa, GPIb, GPIc, GPIIa, GPIX, GPIIb/IIIa, GPIa/IIa, GPIV, GPIc/IIa, GPIb/IX.

37. An agent of paragraph 35 wherein the second portion comprises an amino acid sequence derived from a platelet receptor other than GPVI, or a sequence having homology to at least part of such sequence.

38. An agent of paragraph 35 wherein the second portion:
is functional to interfere with, modulate or confer the activity of platelet derived growth factor (PDGF), glycoprotein IBα, thrombomodulin, vascular epidermal growth factor, transforming growth factor-β1, basic fribroblast growth factor, angiotensin II, factor VIII, von Willebrand factor; or comprises a protein kinase inhibitor, an antiproliferative agent, an antimitotic agent, an antibiotic, an antimetabolite, an anticoagulant, a fibrolytic agent, an antimigratory agent, an antisecretory agent, an anti-inflammatory agent, a non-steroidal agent, an angiogenic agent, an anti-angiogenic agent, an immunosuppressive agent, a pyrimidine analogue, a purine analogue, or a spleen tyrosine kinase (SYK) inhibitor.

39. An agent of paragraph 35 wherein the second portion confers inhibitor activity against platelet adhesion, platelet aggregation or coagulation.

40. An agent of paragraph 35 wherein the second portion comprises an amino acid sequence derived from platelet derived growth factor (PDGF), a platelet receptor protein (e.g. a platelet-bound glycoprotein) hirudin, thrombomodulin, vascular epidermal growth factor, transforming growth factor-1, basic fibroblast growth factor, angiotensin II, factor VIII, von Willebrand factor, tick anticoagulant protein (TAP) or nematode anticoagulant protein (NAP), or a sequence having homology to at least part of such sequence.

41. An agent of paragraph 35 wherein the second portion comprises an imaging agent, for example a radio-imaging agent, e.g. Tc^{99m}.

42. An agent of any of paragraphs 35 to 41 which comprises at least one further portion which is capable of binding to a site other than a GPVI-binding domain and/or which comprises a therapeutic or diagnostic moiety.

43. An agent of any of paragraphs 35 to 42, wherein the first portion is capable of binding to collagen.

44. An agent of paragraph 43, wherein the first portion is capable of binding to collagen I and collagen III.

45. An agent of any of paragraphs 35 to 44, wherein the first portion is capable of binding to vitronectin.

46. An agent of any of paragraphs 35 to 45, wherein the first portion is capable of binding to fibronectin.

47. An agent of any of paragraphs 35 to 42 wherein the first portion is capable of binding to a plurality of types of intravascular GPVI-binding domains, for example to collagen and to one or both of vitronectin and fibronectin.

48. An agent of paragraph 47, wherein the first portion is a distributed portion having spaced apart regions having different binding functions.

49. An agent which is functional to bind to collagen and to one or both of vitronectin and fibronectin, and which further has at least one other therapeutic or diagnostic function.

50. The agent of any of paragraphs 35 to 49 which is an antibody product having plural specificities, each for a different GPVI-binding domain.

51. The agent of paragraph 50 wherein the antibody product is specific for at least two substrates selected from collagen, vitronectin and fibronectin.

52. The agent of paragraph 51 wherein the antibody product is specific for collagen, vitronectin and fibronectin.

53. The agent of any of paragraphs 50 to 52 wherein the antibody product additionally has binding specificity for one or more ligands for other platelet receptors.

54. An agent which is capable of interfering with a plurality of platelet-blood vessel interactions, of which at least one interaction involves GPVI.

55. An agent of paragraph 54 wherein said interactions include interaction with vitronectin.

56. An agent of paragraph 54 wherein said interactions include interaction with fibronectin.

57 An agent of any of paragraphs 54 to 56 which additionally has at least one other therapeutic and/or diagnostic function, for example has a feature recited in any of paragraphs 36 to 41.

58. An agent which is capable of inhibiting GPVI interaction with collagen and with a molecule selected from the group consisting of vitronectin, fibronectin and combinations thereof, the agent further having at least one other activity selected from radioactivity, visualisation, and anti-thrombotic, anti-coagulant and/or anti-platelet activity.

59. The agent of paragraph 58 which is capable of binding to GPVI and vitronectin.

60. The agent of paragraph 58 which is capable of binding to GPVI and fibronectin.

61. The agent of paragraph 58 which is capable of binding GPVI and vitronectin and fibronectin.

62. The agent of any paragraphs 58 to 61 which is a diabody.

63. The agent of paragraph 61 which is a triabody.

64. The agent of any of paragraphs 58 to 61 which comprises a bi-specific antibody or fragment thereof.

65. A pharmaceutical or, as applicable, diagnostic formulation comprising an agent of any preceding paragraph.

66. An indwelling, optionally intravascular, device having a coating or impregnate comprising an agent which inhibits interaction between GPVI and a protein selected from collagen, vitronectin and fibronectin, and combinations thereof.

67. The intravascular device of paragraph 66, wherein said coating further comprises a polymer, optionally selected from the group consisting of a bioabsorbable polymer, a biostable polymer, and combinations thereof.

68. The device of paragraph 66 or paragraph 67, wherein said intravascular device selected from a stent, a vascular catheter, a vascular shunt, a balloon catheter, an autologous venous/arterial graft, a prosthetic venous/arterial graft and a guidewire.

69. The device of any of paragraphs 66 to 68, wherein said agent is capable of inhibiting binding of GPVI to collagen.

70. The device of paragraph 69, wherein said agent is capable of inhibiting binding of GPVI to collagen and to a molecule selected from fibronectin, vitronectin and combinations thereof.

71. The device of paragraph 70, wherein the agent is capable of inhibiting GPVI binding to collagen, fibronectin and vitronectin.

72. The device of any of paragraphs 66 to 71, wherein said agent comprises an amino acid sequence of or comprised in an extracellular domain of GPVI, or a variant thereof that is functional for binding to collagen and to at least one protein selected from fibronectin and vitronectin.

73. The device of any of paragraphs 66 to 71, wherein the agent is a fusion protein comprising an amino acid sequence of or comprised in an extracellular domain of GPVI, or a variant thereof that is functional for binding to collagen, fibronectin and vitronectin.

74. The device of any of paragraphs 66 to 70 wherein the agent is as defined by any of paragraphs 1 to 64.

75. The device of any of paragraphs 66 to 73, which is adapted for said agent to be released from the device when implanted.

76. The device of any of paragraphs 66 to 75, wherein said coating contains one or more further bioactive agents.

77. The intravascular device of paragraph 76, wherein said one or more agents are selected from the group consisting of protein kinase inhibitors, antiproliferative agents, antimitotic agents, antibiotics, antimetabolites, anticoagulants, fibrolytic agents, antimigratory agents, antisecretory agents, anti-inflammatory agents, non-steroidal agents, angiogenic agents, anti-angiogenic agents, immunosuppressive agents, pyrimidine analogues, purine analogues, spleen tyrosine kinase (SYK) inhibitors and combinations thereof.

78. A method of inhibiting or preventing restenosis, thrombosis, atherogenesis, atheroprogression, atherosclerosis, and/or vascular inflammation in a patient, said method comprising implanting in said patient an intravascular device comprising a direct or indirect GPVI inhibitor adapted to be exposed and/or released when the device is implanted.

79. The method of paragraph 78 wherein the device is made using or has a coating which comprises a polymer impregnated with the inhibitor.

80. The method of paragraph 78 or paragraph 79 wherein said intravascular device is selected from a stent, a vascular shunt, a vascular catheter, a balloon catheter, an autologous venous/arterial graft, a prosthetic venous/arterial graft and a guidewire.

81. The method of any of paragraphs 78 to 80, wherein said GPVI inhibitor blocks the adhesion of platelets to the lumen of a blood vessel.

82. The method of any of paragraphs 78 to 81, wherein said GPVI inhibitor is capable of inhibiting the binding of GPVI to collagen.

83. The method of paragraph 82, wherein said GPVI inhibitor is capable of inhibiting binding of GPVI to collagen and to a molecule selected from vitronectin, fibronectin and combinations thereof.

84. The method of any of paragraphs 78 to 80 wherein the GPVI inhibitor is as defined by any of paragraphs 1 to 64.

85. A method for treating a patient suffering from, or at risk of suffering from, a disorder characterised by an interaction between GPVI and fibronectin and/or vitronectin, comprising administering an effective amount of an agent which inhibits interaction between GPVI and a protein selected from the group consisting of fibronectin, vitronectin and combinations thereof.

86. A method of paragraph 85, wherein the agent inhibits interaction between GPVI and both of vitronectin and fibronectin.

87. A method of paragraph 85 or 86 wherein the disorder is initiation of atherosclerosis.

88. A method for secondary prophylaxis of a patient who has suffered, or is at risk or suspicion of having suffered, an atherosclerotic event, comprising administering to the patient an effective amount of an agent which inhibits interaction between GPVI and a protein, e.g. two proteins, selected from the group consisting of collagen, fibronectin, vitronectin and combinations thereof.

89. A method of paragraph 88 wherein said patient suffered, or is at risk or suspicion of having suffered, from a disorder selected from the group consisting of thrombosis, myocardial infarction, strokes, transient ischemic attack, occlusive peripheral vascular disease, occlusion of peripheral arteries and complications thereof.

90. A method of paragraphs 88 or 89, wherein the agent inhibits interaction between GPVI and vitronectin.

91. A method of any of paragraphs 88 to 90, wherein the agent inhibits interaction between GPVI and fibronectin.

92. A method of any of paragraphs 85 to 92, wherein the agent is as defined by any of paragraphs 1 to 64 or A1 to A29, A33 to A64 and A68 to A105, optionally wherein the agent (i) is or, alternatively, (ii) is not a fusion protein defined in paragraph 15.

93. A method of treating a patient who has suffered a cardiovascular event comprising administering to the patient an effective amount of a direct or indirect GPVI inhibitor for at least three months after said cardiovascular event.

94. The method of paragraph 93 wherein said GPVI inhibitor inhibits the interaction between GPVI and a protein selected from the group consisting of fibronectin, vitronectin, collagen and combinations thereof.

95. The method of paragraph 94 or paragraph 95, wherein the mode of administration is selected from the group consisting of parenteral, e.g. subcutaneous, intravenous or interarterial, administration and oral administration.

96. The method of any of paragraphs 93 to 95 wherein the GPVI inhibitor is a defined by way of paragraphs 1 to 64 or A1 to A29, A33 to A64 and A68 to A105, optionally wherein the agent (i) is or, alternatively, (ii) is not a fusion protein defined in paragraph 15.

97. The method of any of paragraphs 93 to 96 wherein the GPVI inhibitor is administered for a period of up to six months after said cardiovascular event.

98. The method of any of paragraphs 93 to 97 wherein the cardiovascular event comprises rupture or fissure of atherosclerotic plaque.

99. A method of treating a patient suffering from, or suspected to be suffering from, an atherosclerotic disorder, comprising administering locally to a site or suspected site of atherosclerotic disorder an agent which inhibits binding of GPVI to the site.

100. A method of paragraph 99 wherein the atherosclerotic disorder is an atherosclerotic plaque.

101. A method of paragraph 100 wherein the atherosclerotic plaque is ruptured or fissured.

102. A method of any of paragraphs 99 to 101 wherein the agent is delivered through a catheter.

103. A method of any of paragraphs 99 to 102 wherein the agent is as defined in any of paragraphs 1 to 64 or A1 to A29, A33 to A64 and A68 to A105, optionally wherein the agent (i) is or, alternatively, (ii) is not a fusion protein defined in paragraph 15.

104. A method for treating a patient having, or identified as having, at least one or a combination of risk factors for the formation of atherosclerotic lesions, comprising administering an effective amount of an agent which inhibits interaction between GPVI and a protein selected from the group consisting of fibronectin, vitronectin and combinations thereof.

105. A method of treating a patient who has been identified as having one or more risk factors associated with developing atherosclerosis, said patient having not yet developed advanced atherosclerotic plaques, wherein said patient is considered to have a risk score of 45 or greater according to the PROCAM study, said method comprising administering an agent which inhibits binding of GPVI to a molecule selected from vitronectin, fibronectin and combinations thereof.

106. A method of paragraph 105 where in the risk score is at least 50.

107. A method for preventing or retarding atherosclerotic cardiovascular disease in a patient who is not displaying clinical symptoms of atherosclerosis, the method comprising administering to the patient an agent which inhibits binding of GPVI to a molecule selected from vitronectin, fibronectin and combinations thereof.

108. The method of paragraph 107 wherein the patient has a 10-year risk of fatal cardiovascular disease according to the SCORE project of at least 3%.

109. The method of paragraph 108 wherein the risk is at least 5%.

110. The method of paragraph 108 wherein the risk is at least 10%.

111. A method for the primary prevention of atherosclerotic cardiovascular disease in a patient, the method comprising administering to the patient an agent which inhibits binding of GPVI to a molecule selected from vitronectin, fibronectin and combinations thereof.

112. A method for treating patient having, or identified as having, a marker of vascular inflammation, comprising administering to the patient an agent which inhibits binding of GPVI to a molecule selected from vitronectin, fibronectin and combinations thereof.

113. The method of paragraph 112 wherein the patient is asymptomatic.

114. A method for treating atherosclerosis in a patient, comprising administering to the patient an agent capable of binding to a plurality of GPVI-binding sites at a frequency of once a day or less.

115. The method of paragraph 114 wherein the agent is administered at a frequency of at most once every other day.

116. The method of paragraph 114 wherein the agent is administered at a frequency of at most once every 48 hours.

117. The method of paragraph 114 wherein the agent is administered at a frequency of at most once every 72 hours, e.g. at most once every 96 hours.

118. The method of paragraph 114 wherein the agent is administered at a frequency of at most once a week.

119. The method of any of paragraphs 114 to 118 where the GPVI-binding sites comprise two or more different ligands for GPVI.

120. The method of any of paragraphs 114 to 118 where the GPVI-binding sites comprise collagen and fibronectin.

121. The method of any of paragraphs 114 to 118 where the GPVI-binding sites comprise collagen and vitronectin.

122. The method of any of paragraphs 114 to 118 where the GPVI-binding sites comprise collagen, fibronectin and vitronectin.

123. The method of any of paragraphs 114 to 118 wherein the agent is as defined in any of paragraphs 1 to 64.

124. The method according to any of paragraphs 78 to 122 wherein the patient is diabetic.

125. A pharmaceutical formulation comprising:
an agent which inhibits GPVI interaction with fibronectin; and
another antithrombotic drug.

126. A pharmaceutical formulation comprising:
an agent which inhibits GPVI interaction with fibronectin and vitronectin; and
another antithrombotic drug

127. A pharmaceutical formulation comprising:
an agent which inhibits GPVI interaction with vitronectin; and
another antithrombotic drug.

128. The pharmaceutical formulation of any of paragraphs 125 to 127 wherein said agent further inhibits GPVI interaction with collagen.

129. The pharmaceutical formulation of any of paragraphs 125 to 128 wherein the agent is as defined in any of paragraphs 1 to 64 or A1 to A29, A33 to A64 and A68 to A105, optionally wherein the agent (i) is or, alternatively, (ii) is not a fusion protein defined in paragraph 15.

130. A method of treating in a subject a disorder selected from thrombosis, atherosclerosis and combinations thereof, comprising administering to the subject effective amounts of (i) an agent which inhibits GPVI interaction with fibronectin; and (ii) another antithrombotic drug.

131. A method of treating in a subject a disorder selected from thrombosis, atherosclerosis and combinations thereof, comprising administering to the subject effective amounts of (i) an agent which inhibits GPVI interaction with fibronectin and vitronectin; and (ii) another antithrombotic drug.

132. A method of treating in a subject a disorder selected from thrombosis, atherosclerosis and combinations thereof, comprising administering to the subject effective amounts of (i) an agent which inhibits GPVI interaction with vitronectin; and (ii) another antithrombotic drug.

133. The method of any of paragraphs 130 to 132 wherein said agent further inhibits GPVI interaction with collagen.

134. The method of any of paragraphs 130 to 133 wherein said agent comprises an amino acid sequence of or comprised in an extracellular domain of GPVI, or a variant thereof that is functional for binding to at least one of fibronectin and vitronectin.

135. A method of treating in a subject a disorder selected from thrombosis, atherosclerosis and combinations thereof, comprising administering to the subject effective amounts of:
A) an agent which comprises an amino acid sequence encoded by (i) a nucleic acid sequence which encodes a wild type GPVI extracellular domain; (ii) a nucleic acid sequence which hybridises to said nucleic acid sequence (i) and which encodes a polypeptide that is capable of binding to a plurality of types of GPVI-binding sites of a dysfunctional, inflamed or atherosclerotic blood vessel area; or (iii) a nucleic acid sequence which differs from said nucleic acid sequence (i) by virtue of the degeneracy of the genetic code; and
(B) another antithrombotic drug which acts by a mechanism not involving binding to a GPVI-binding site.

136. A method of treating in a subject a disorder selected from thrombosis, atherosderosis and combinations thereof, comprising administering to the subject effective amounts of:
(i) a fusion protein comprising (a) an extracellular domain of GPVI or a variant thereof that is functional for binding to collagen; and (b) an Fc domain of an immunoglobulin or a functional conservative part thereof, the extracellular domain and the Fc domain being fused via a linker characterised by the amino acid sequence Gly-Gly-Arg; and
(ii) another antithrombotic drug, said other antithrombotic drug having a mechanism of action which does not interfere with binding between GPVI and a ligand for GPVI.

137. The method of any of paragraphs 130 to 136 wherein the other antithrombotic drug is selected from the group consisting of platelet adhesion inhibitors and platelet aggregation inhibitors.

138. The method of any of paragraphs 130 to 136 wherein the other antithrombotic drug is a platelet aggregation inhibitor.

139. The method of paragraph 138 wherein the platelet aggregation inhibitor is selected from the group consisting of thromboxane synthesis inhibitors and P2Y ADP receptor antagonists.

140. The method of paragraph 139 wherein the thromboxane synthesis inhibitor is aspirin (acetyl salicylic acid).

141. The method of paragraph 139 wherein the P2Y ADP receptor antagonist is dopidogrel.

142. The method of any of paragraphs 130 to 138 wherein a plurality of said other antithrombotic drugs are administered, each having a different mechanism of action.

143. The method of paragraph 142 wherein said other antithrombotic drugs comprise a thromboxane synthesis inhibitor and a P2Y ADP receptor antagonists.

144. A method for treating thrombosis in a subject, comprising administering to the subject effective amounts of an agent which interferes with interaction between GPVI and a blood vessel wall area, and one or more other agents selected from the group consisting of anti-coagulants and anti-platelet drugs which act by a mechanism not involving GPVI.

145. The method of paragraph 144 wherein said one or more other agents include at least one anti-platelet drug selected from aspirin (acetyl salicylic acid), clopidogrel, dipyridamole, GPIIb/IIIa blockers (e.g. abciximab, tirofiban and eptifibatide).

146. The method of paragraph 144 wherein said one or more other agents include at least one coagulation enzyme inhibitor, e.g. an inhibitor of thrombin, Factor IXa or Factor X.

147. The method of paragraph 146 wherein the coagulation enzyme inhibitor is a direct thrombin inhibitor.

148. The method of paragraph 146 wherein the coagulation enzyme inhibitor is a low molecular weight thrombin inhibitor, for example melagatran or TRI 50c, or a salt or prodrug of either.

149. The method of any of paragraphs 130 to 148, wherein said agent and said other drug(s) are administered at substantially the same time.

150. The method of any of paragraphs 130 to 148, wherein said agent and said other drug(s) are administered at different times.

151. The method of any of paragraphs 130 to 150, wherein said agent is administered at periods of approximately two days or longer.

152. The method of any of paragraphs 130 to 151, wherein the treatment is prophylactic.

153. An agent which inhibits GPVI interaction with fibronectin.

154. An agent according to paragraph 153 which inhibits GPVI binding to fibronectin.

155. An agent according to paragraph 153 or paragraph 154, which binds to fibronectin.

156. An agent according to paragraph 153 or paragraph 154, which binds to fibronectin at a GPVI binding site.

157. An agent according to paragraph 153 or paragraph 154 which binds to GPVI.

158. An agent according to paragraph 157 which binds to GPVI at a fibronectin binding site.

159. An agent which inhibits GPVI interaction with vitronectin.

160. An agent according to paragraph 159 which inhibits GPVI binding to vitronectin.

161. An agent according to paragraph 159 or paragraph 160 which binds to GPVI.

162. An agent according to paragraph 161 which binds to GPVI at a vitronectin binding site.

163. An agent according to paragraph 159 or paragraph 160, which binds to vitronectin.

164. An agent according to paragraph 163 which binds to vitronectin at a GPVI binding site.

165. An agent according to any preceding claim which is selected from an antibody, an antibody fragment, a protein, a polypeptide, a fusion protein, an aptamer and a compound.

166. An agent of paragraph 165, wherein the antibody or fragment thereof is a monoclonal antibody, a polyclonal antibody, a chimeric antibody, a human antibody or a humanized antibody and the fragment is a fragment of such an antibody.

167. An agent of paragraph 165 or paragraph 166, wherein the antibody fragment is a Fab fragment, a F(ab′)₂ fragment, a scFv, a Fv fragment or a single domain antibody.

168. A nucleic acid comprising a nucleic acid sequence, which sequence encodes an agent as claimed in any preceding claim when the agent is an antibody, a fragment thereof, a fusion protein, a polypeptide or a protein.

169. An expression vector comprising a nucleic acid of paragraph 168 and associated regulatory sequences necessary for expression of a protein or polypeptide in a host cell.

170. A host cell comprising a nucleic acid according to paragraph 168 or a vector according to paragraph 169.

171. Use of fibronectin in a binding assay for identifying an agent capable of inhibiting interaction of GPVI and fibronectin.

172. Use according to paragraph 171, wherein the agent is capable of inhibiting binding of GPVI to fibronectin.

173. Use of vitronectin in a binding assay for identifying an agent capable of inhibiting interaction of GPVI and vitronectin.

174. Use according to paragraph 173, wherein the agent is capable of inhibiting binding of GPVI to fibronectin.

175. A method of inhibiting platelet aggregation, comprising contacting platelets with an effective amount of the agent according to any of paragraphs 153 to 167.

176. A method according to paragraph 175, wherein the platelets are in vitro.

177. A method according to paragraph 175, wherein the platelets are in vivo.

178. A method of treating a disease or disorder associated with pathological interaction between platelet-bound GPVI and fibronectin comprising administering an agent which inhibits the binding of platelet bound GPVI to fibronectin to a subject with the disease or disorder or at risk of developing the disease or disorder.

179. A method of treating a disease or disorder associated with an pathological interaction between platelet-bound GPVI and vitronectin comprising administering an agent which inhibits the binding of platelet bound GPVI to vitronectin to a subject with the disease or disorder or at risk of developing the disease or disorder.

180. A method according to paragraph 178 or paragraph 179, wherein the treatment istherapeutic or prophylactic and wherein the a disease or disorder is selected from acute coronary syndromes, cardiovascular thrombosis, cerebrovascular thrombosis, unstable angina, stable angina, angina pectoris, embolus formation, deep vain thrombosis, hemolytic uremic syndrome, hemolytic anemia, acute renal failure, thrombolytic complications, thrombotic thrombocytopenic purpura, disseminated intravascular comgelopathy, thrombosis, coronary heart disease, thromboembolic complications, myocardial infarction, restenosis, and atrial thrombosis formation in atrial fibrillation, chronic unstable angina, transient ischemic attacks and strokes, peripheral vascular disease, arterial thrombosis, pre-eclampsia, embolism, restenosis and/or thrombosis following angioplasty, carotid endarterectomy, anastomosis of vascular grafts, and chronic exposure to cardiovascular devices.

181. A method according to paragraph 180, wherein the treatment is for treating or preventing thromboembolism and reocculsion occurring during and after thrombolytic therapy, after angioplasty, and after coronary artery bypass.

182. A method of preventing or retarding initiation and/or progression of atherosclerotic lesions in a subject with the atherosclerotic lesions or at risk of developing atherosclerotic lesions.

183. A method of preventing or retarding initiation and/or progression of atherosclerotic lesions in a subject substantially free of active atherosclerotic lesions.

184. A method of treating or reducing atherosclerotic lesions in a subject.

185. Use of an agent according to any one of paragraphs 153 to 167 for the manufacture of a medicament for the treatment of a disease or disorder associated with pathological interaction between platelet-bound GPVI and vitronectin.

186. Use of an agent according to any one of paragraphs 153 to 167 for the manufacture of a medicament for the treatment of a disease or disorder associated with pathological interaction between platelet-bound GPVI and fibronectin.

187. Use according to claim 33, wherein the treatment is therapeutic or prophylactic and wherein the a disease or disorder is selected from acute coronary syndromes, cardiovascular thrombosis, cerebrovascular thrombosis, unstable angina, stable angina, angina pectoris, embolus formation, deep vain thrombosis, hemolytic uremic syndrome, hemolytic anemia, acute renal failure, thrombolytic complications, thrombotic thrombocytopenic purpura, disseminated intravascular comgelopathy, thrombosis, coronary heart disease, thromboembolic complications, myocardial infarction, restenosis, and atrial thrombosis formation in atrial fibrillation, chronic unstable angina, transient ischemic attacks and strokes, peripheral vascular disease, arterial thrombosis, pre-eclampsia, embolism, restenosis and/or thrombosis following angioplasty, carotid endarterectomy, anastomosis of vascular grafts, and chronic exposure to cardiovascular devices.

188. A method according to paragraph 187, wherein the medicament for treating or preventing thromboembolism and reclusion occurring during and after thrombolytic therapy, after angioplasty, and after coronary artery bypass.

189. A method of in vitro screening for inhibitors of binding of glycoprotein VI to fibronectin, comprising
(i) contacting a substrate having exposed fibronectin with a fibronectin-binding product of any of paragraphs 1 to 64 or 153 to 167 in the presence of a test compound;
(ii) performing step (i) in the absence of the test compound, to allow binding of the product to the surface;
(iii) measuring the amount of said fusion protein bound to said substrate in the absence and in the presence of said test compound;
(iv) identifying a test compound as inhibitor if binding of said fusion protein to said surface is less in the presence of said test compound as compared to the absence of the test compound; and
(v) optionally determining the functional effect of said inhibitor on platelet aggregation and/or platelet activation.

190. A method of in vitro screening for inhibitors of binding of glycoprotein VI to vitronectin, comprising
(i) contacting a substrate having exposed vitronectin with a vitronectin-binding product of any of paragraphs 1 to 64 or 153 to 167 in the presence of a test compound;
(ii) performing step (i) in the absence of the test compound, to allow binding of the product to the surface;
(iii) measuring the amount of said fusion protein bound to said substrate in the absence and in the presence of said test compound;
(iv) identifying a test compound as inhibitor if binding of said fusion protein to said surface is less in the presence of said test compound as compared to the absence of the test compound; and
(v) optionally determining the functional effect of said inhibitor on platelet aggregation and/or platelet activation.

191. A method for treating a patient suffering, or at risk of suffering, a cardiovascular disorder, comprising administering to the patient a plurality of agents, each targeting a different binding mode, for a component of the blood.

192. The method of paragraph 191 wherein at least one of, and optionally all of, the binding modes is/are between the blood component and a blood vessel.

193. The method of paragraph 191 or 192 wherein the blood component comprises platelets.

194. The method of any of paragraphs 191 to 193 wherein the blood component comprises monocytes.

195. The method of any of paragraphs 191 to 1943 whereinthere are administered two or more agents, e.g. two or three agents, each of which interferes with interaction of GPVI with a different one of vitronectin, fibronectin and collagen.

196. A product comprising a plurality of active agents, each agent being capable of interrupting a different mode of binding, for example different receptor-ligand interaction, of a blood component.

197. The method or product of any of paragraphs 191 to 196 wherein the plurality of agents are all antibody products, for example antibody fragments.

198. The method or product of any of paragraphs 191 to 196 wherein the plurality of agents are all single domain antibodies or are all domain antibodies.

199. A method for diagnosing atherosclerotic cardiovascular disease in a patient who is not displaying clinical symptoms of atherosclerosis, the method comprising administering to the patient an agent which inhibits binding of GPVI to a molecule selected from vitronectin, fibronectin and combinations thereof and which comprises a moiety to enable visualisation or locating of the moiety in vivo.

200. A method of paragraph 199 wherein said moiety comprises a binding partner, e.g. antibody product, or a radionuclide.

### REFERENCES

1. Ruggeri, Z. M. Platelets in atherothrombosis. Nat Med8, 1227-1234 (2002).
2. Gawaz, M., Neumann, F J., Dickfeld, T., Koch, W., Laugwitz, K. L., Adelsberger, H., Langenbrink, K., Page, S., Neumeler, D., Schömig, A., Brand, K. Vitronectin receptor (alpha(v) beta3) mediates platelet adhesion to the luminal aspect of endothelial cells: implications for reperfusion in acute myocardial infarction. Circulation 96, 1809-1818 (1997).
3. Ruggeri, Z. M. Von Willebrand factor, platelets and endothelial cell interactions. J Thromb Haemost 1, 1335-1342 (2003).
4. Sachals, B. S. Platelet-endothelial interactions in atherosclerosis. Current Atherosclerosis Reports 3, 412-416 (2001).
5. George, J. N. Platelets. Lancet 355, 1531-1539 (2000).
6. Springer, T. A. Traffic signals for lymphocyte recirculation and leukocyte emigration: the multistep paradigm. Cell 76, 301-314 (1994).
7. Clemetson, J. M., Polgar, 3., Magnenat, E., Wells, T. N., Clemetson, K. J. The platelet collagen receptor glycoprotein VI is a member of the immunoglobulin superfamily closely related to FcalphaR and the natural killer receptors. J Biol Chem 274, 29019-24 (1999).
8. Kuljpers, M. J., Schulte, V., Bergmeler, W., Lidhout, T., Brakebusch, C., Offermanns, S., Fässler, R., Heemskerk, J. W., Nieswandt, B. Complementary roles of glycoprotein VI and alpha2beta1 integrin in collagen-induced thrombus formation in flowing whole blood ex vivo. FASEB J 17, 685-687 (2003).
9. Massberg, S., Gawaz, M., Grüner, S., Schulte, V., Konrad, I., Zohlnhöfer, D., Heinzmann, U., Nieswandt, B. A crucial role of glycoprotein VI for platelet recruitment to the injured arterial wall in vivo. J Exp Med 197, 41-49 (2003).
10. Nieswandt, B., Watson, S. P. Platelet-collagen interaction: is GPVI the central receptor? Blood 102, 449-461 (2003).
11. Miura, Y., Takahashi, T., Jung, S. M., Morol, M. Analysis of the interaction of platelet collagen receptor glycoprotein VI (GPVI) with collagen. A dimeric form of GPVI, but not the monomeric form, shows affinity to fibrous collagen. J Biol Chem 277, 46197-46204 (2002).
12. Massberg, S., Konrad, I., Bütmann, A., Schulz, C., Mûnch, G., Peluso, M., Lorenz, M., Schneider, S., Besta, F., Müller, I., Hu, B., Langer, H., Kremmer, E., Rudellus, M., Heinzmann, U., Ungerer, M., Gawaz, M. Soluble glycoprotein VI inhibits platelet adhesion and aggregation to the injured vessel wall in vivo. FASEB (2003) December 4 [Epub ahead of print].
13. Frenette, P. S., Wagner, D. D. Adhesion molecules—Part II. Blood vessels and blood cells. N Engl J Med 335, 43-45 (1996).
14. Cheresh, D. A., Berliner, A. S., Vicente, V., Rugged, Z. M. Recognition of distinct adhesive sites on fibrinogen by related integrins on platelets and endothelial cells. Cell 158, 945-953 (1989).
15. Gawaz, M. P., Loftus, J. C., Bait, M. L., Frojmovic, M. M., Plow, E. F., Ginsberg, M. H. Ligand bridging mediates integrin alpha IIb beta 3 (platelet GPIIB-IIIA) dependent homotypic and heterotypic cell-cell interactions. J Clin Invest 88, 1128-1134 (1991).
16. Frenette, P. S., Johnson, R. C., Hynes, R. O., Wagner, D. D. Platelets roll on stimulated endothelium in vivo: an interaction mediated by endothelial P-selectin. Proc Natl Acad Sci US A 92, 7450-7454 (1995).
17. Frenette, P. S., Moyna, C., Hartwell, D. W., Lowe, J. B., Hynes, R. O., Wagner, D. D. Platelet-endothelial interactions in inflamed mesenteric venules. Blood 91, 1318-1324 (1998).
18. Bombell, T., Schwartz, B. R., Harlan, J. M. Adhesion of activated platelets to endothelial cells: evidence for a GPIIbIIIa-dependent bridging mechanism and novel roles for endothelial intercellular adhesion molecule 1 (ICAM-1), alphavbeta3 integrin, and GPIbalpha. J Exp Med 187, 329-333 (1998).
19. Massberg, S., Enders, G., Matos, F. C., Matos, F. C., Tomic, L. I., Leiderer, R., Elsenmenger, S., Messmer, K., Krombach, F. Fibrinogen deposition at the postischemic vessel wall promotes platelet adhesion during ischemia-reperfusion in vivo. Blood 94, 3829-3838 (1999).
20. Romo, G. M., Dong, J. F., Schade, A. J., Gardiner, E. E., Kansas, G. S., Li, C. Q., McIntire, L. V., Berndt, M. C., Lopez, J. A. The glycoprotein Ib-IX-V complex is a platelet counterreceptor for P-selectin. J Exp Med 190, 803-814 (1999).
21. Zanetti, A., Conforti, G., Hess, S., Martin-Padura, I., Ghibaudi, E., Preissner, K. T., Dejana, E. Clustering of vitronectin and RGD peptides on microspheres leads to engagement of integrins on the luminal aspect of endothelial cell membrane. Blood 84, 1116-1123 (1994).
22. Andre, P., Denis, C. V., Ware, J., Saffaripour, S., Hynes, R. O., Ruggeri, Z. M., Wagner, D. D. Platelets adhere to and translocate on von Willebrand factor presented by endothelium in stimulated veins. Blood 96, 3322-3328 (2000).
23. Shih, P. T., Elices, M. J., Fang, Z. T., Ugarova, T. P., Strahl, D., Territo, M. C., Frank, J. S., Kovach, N. L., Cabanas, C., Berliner, J. A., Vora, D. K. Minimally modified low-density lipoprotein induces monocyte adhesion to endothelial connecting segment-1 by activating beta1 integrin. J Clin Invest 103, 613-625 (1999).
24. Hatley, M. E., Srinivasan, S., Reilly, K. B., Bolick, D. T., Hedrick, C. C. Increased production of 12/15 lipoxygenase eicosanoids accelerates monocyte/endothelial interactions in diabetic db/db mice. J Biol Chem 278, 25369-25375 (2003).
25. Polanowska-Grabowska, R., Gear, A. R. High-speed platelet adhesion under conditions of rapid flow. Proc Natl Acad Sci USA 89, 5754-5758 (1992).
26. Savage, B., Almus-Jacobs, F., Ruggeri, Z. M. Specific synergy of multiple substrate-receptor interactions in platelet thrombus formation under flow. Cell 94, 6.57-66 (1988).
27. Massberg, S., Brand, K., Gruner, S., Page, S., Müller, E., Müller, I., Bergmeler, W., Richter, T., Lorenz, M., Konrad, I., Nieswandt, B., Gawaz, M. A critical role of platelet adhesion in the initiation of atherosclerotic lesion formation. J Exp Med 196, 887-896 (2002).
28. Nieswandt, B., Bergmeler, W., Schulte, V., Rackebrandt, K., Gessner, J. E., Zirngibl, H. Expression and function of the mouse collagen receptor glycoprotein VI is strictly dependent on its association with the FcRgamma chain. J Biol Chem 275, 23998-4002 (2000).
29. Nieswandt, B., Schulte, V., Bergmeler, W., Mokhatari-Nejad, R., Rackebrandt, K., Cazenave, J. P., Ohimann, P., Gachet, C., Zirngibl, H. Long-term antithrombotic protection by in vivo depletion of platelet glycoprotein VI in mice. J Exp Med 193, 459-469 (2001).
30. Moers, A., Nieswandt, B., Massberg, S., Wettschureck, N., Grüner, S., Konrad, I., Schulte, V., Aktas, B., Gratacap, M. P., Simon, M. I., Gawaz, M., Offermanns, S. G (13) is an essential mediator of platelet activation in hemostasis and thrombosis. Nat Med 9, 1418-22 (2003).

1-88. (canceled)

89. A method for inhibiting or preventing a disorder selected from restenosis, thrombosis, atherogenesis, atheroprogession, atherosclerosis and/or vascular inflammation in a patient, the method comprising administering a therapeutically effective amount of an inhibitor of interaction between GPVI and a protein selected from vitronectin, fibronectin and combinations thereof to the patient; or for treating restenosis, the method comprising administering a therapeutically effective amount of a said inhibitor to a subject having restenosis or at risk of developing it.

90. The method of claim 89, which is for the inhibition or prevention of the disorder following percutaneous trans-luminal angioplasty.

91. The method of claim 89, wherein the inhibitor is administered locally to a site or suspected site of the disorder.

92. The method of claim 89, wherein the inhibitor is: a polypeptide comprising a sequence of or contained in an extracellular domain of GPVI or a function conservative variant of fragment thereof, the sequence having a homology of at least 90% with an extracellular domain of GPVI; an antibody; an antibody fragment; or an aptamer.

93. The method of claim 89, wherein the fusion protein comprises sequentially in an N-terminus to C-terminus direction, a first amino acid sequence, a second amino acid sequence and a third amino acid sequence, wherein said first amino acid sequence comprises:
A) i) an amino acid sequence encoded by a nucleic acid sequence of bases 1 to 807 of SEQ ID NO: 2;
ii) an amino acid sequence encoded by a nucleic acid sequence which hybridises to bases 1 to 807 of SEQ ID NO: 2 and which codes for a polypeptide which binds to collagen; or
iii) an amino acid sequence encoded by a nucleic acid sequence which differs from bases 1 to 807 of SEQ ID NO: 2 by virtue of the degeneracy of the genetic code and which binds to collagen;
wherein the second amino acid comprises:
B) i) an amino acid sequence encoded by a nucleic acid sequence of bases 808 to 816 of SEQ ID NO: 2;
ii) a 3-mer amino acid sequence containing a hydrophilic amino acid or
iii) an amino acid sequence encoded by a nucleic acid sequence which differs from bases 808 to 816 of SEQ ID NO: 2 by virtue of the degeneracy of the genetic code; and wherein the third amino acid sequence comprises:
C) i) an amino acid sequence encoded by a nucleic acid sequence of bases 817 to 1515 of SEQ ID. NO: 2;
ii) an amino acid sequence encoded by a nucleic acid sequence which hybridises to bases 817 to 1515 of SEQ ID NO: 2 and which codes for a polypeptide which is functional as an Fc domain of an immunoglobulin; or
iii) an amino acid sequence encoded by a nucleic acid sequence which differs from bases 817 to 1515 of SEQ ID NO: 2 by virtue of the degeneracy of the genetic code and which is functional as an Fc domain of an immunoglobulin.

94. An indwelling device having a coating or impregnate comprising an agent which inhibits interaction between GPVI and a protein selected from collagen, vitronectin and fibronectin, and combinations thereof.

95. The device of claim 94, wherein said coating further comprises a polymer, optionally selected from the group consisting of a bioabsorbable polymer, a biostable polymer, and combinations thereof.

96. The device of claim 94, wherein said device is a stent, a vascular catheter, a vascular shunt, a balloon catheter, an autologous venous/arterial graft, an anastomosis device, a vascular implant, a prosthetic venous/arterial graft tissue scaffold, a vascular device, or a guidewire.

97. The device of claim 94, wherein said agent is capable of inhibiting binding of GPVI to collagen.

98. The device of claim 97, wherein said agent is capable of inhibiting binding of GPVI to collagen and to a molecule selected from fibronectin, vitronectin and combinations thereof.

99. The device of claim 94, wherein said agent comprises an amino acid sequence of or comprised in an extracellular domain of GPVI, or a variant thereof that is functional for binding to collagen and to at least one protein selected from fibronectin and vitronectin.

100. The device of claim 99, wherein the agent is a fusion protein comprising an amino acid sequence of or comprised in an extracellular domain of GPVI, or a variant thereof that is functional for binding to collagen, fibronectin and vitronectin.

101. The device of claim 94, wherein the agent is a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

102. The device of claim 94, which is adapted for said agent to be released from the device when implanted.

103. A method of inhibiting or preventing restenosis, thrombosis, atherogenesis, atheroprogression, atherosclerosis, and/or vascular inflammation in a patient, said method comprising implanting in said patient an intravascular device comprising a direct or indirect GPVI inhibitor adapted to be exposed and/or released when the device is implanted.

104. The method of claim 103, wherein the device is made using or has a coating which comprises a polymer impregnated with the inhibitor.

105. The method of claim 103, wherein said intravascular device is a stent, a vascular shunt, a vascular catheter, a balloon catheter, an autologous venous/arterial graft, anastomosis device, a vascular implant, a prosthetic venous/arterial graft or a guidewire.

106. The method of claim 103, wherein said GPVI inhibitor blocks the adhesion of platelets to the lumen of a blood vessel.

107. The method of claim 106, wherein said GPVI inhibitor is capable of inhibiting binding of GPVI to collagen and to a molecule selected from vitronectin, fibronectin and combinations thereof.

108. The method of claim 107, wherein the agent is a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

109. A method for treating a patient suffering from, or at risk of suffering from, a disorder characterised by an interaction between GPVI and fibronectin and/or vitronectin, comprising administering an effective amount of an agent which inhibits interaction between GPVI and a protein selected from the group consisting of fibronectin, vitronectin and combinations thereof.

110. A method of claim 109, wherein the agent inhibits interaction between GPVI and both of vitronectin and fibronectin.

111. A method of claim 110, wherein the agent is a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

112. A method of claim 109, wherein the disorder is initiation of atherosclerosis.

113. A method for treating a patient having, or identified as having, at least one or a combination of risk factors for the formation of atherosclerotic lesions, or a patient who has been identified as having one or more risk factors associated with developing atherosclerosis, said patient having not yet developed advanced atherosclerotic plaques, wherein said patient is considered to have a risk score of 45 or greater according to the PROCAM study, the method comprising administering an effective amount of an agent which inhibits interaction between GPVI and a protein selected from the group consisting of fibronectin, vitronectin and combinations thereof.

114. A method for preventing or retarding atherosclerotic cardiovascular disease in a patient who is not displaying clinical symptoms of atherosclerosis, the method comprising administering to the patient an agent which inhibits binding of GPVI to a molecule selected from vitronectin, fibronectin and combinations thereof.

115. The method of claim 114, wherein the patient has a 10-year risk of fatal cardiovascular disease according to the SCORE project of at least 3%.

116. The method of claim 114, wherein the inhibitor is a polypeptide comprising a sequence of or contained in an extracellular domain of GPVI or a function conservative variant of fragment thereof, the sequence having a homology of at least 90% with an extracellular domain of GPVI; an antibody; an antibody fragment; or an aptamer.

117. A method for the primary prophylaxis of atherosclerotic cardiovascular disease in a patient or for treating patient having, or identified as having, a marker of vascular inflammation, the method comprising administering to the patient an agent which inhibits binding of GPVI to a molecule selected from vitronectin, fibronectin and combinations thereof.

118. The method of claim 117, wherein the patient is asymptomatic.

119. The method of claim 117, wherein the agent is a fusion protein comprising:
a) an extracellular domain of GPVI or a variant thereof that is functional for binding to collagen; and
b) an Fc domain of an immunoglobulin or a functional conservative part thereof, the extracellular domain and the Fc domain being fused via a linker characterised by the amino acid sequence Gly-Gly-Arg.

120. A method for diagnosing atherosclerotic cardiovascular disease in a patient who is not displaying clinical symptoms of atherosclerosis, the method comprising administering to the patient an agent which inhibits binding of GPVI to a molecule selected from vitronectin, fibronectin and combinations thereof and which comprises a moiety to enable visualisation or locating of the moiety in vivo.

121. The method of claim 120, wherein said agent: a polypeptide which comprises an amino acid sequence of or comprised in an extracellular domain of GPVI, or a variant thereof that is functional for binding to collagen and to at least one protein selected from fibronectin and vitronectin; an antibody; an antibody fragment; or an aptamer.
